SEQUENCE LISTING (170 sequences)

SEQ ID NO: 1 - protein, 650 aa, Artificial Sequence
  Recombinant form of wild-type human ILT-2
MTPILTVLICLGLSLGPRTHVQAGHLPKPTLWAEPGSVITQGSPVTLRCQGGQETQEYRLYREKKTAPWITRIPQELVKKGQFPIPSITWEHAGRYRCYYGSDTAGRSESSDPLELVVTGAYIKPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRRKATEPPPSQEGPSPAVPSIYATLAIH

SEQ ID NO: 2 - DNA, 2984 bp, Artificial Sequence
  DNA encoding recombinant form of wild-type human ILT-2
gaggaggaacagaaaagaaaagaaaagaaaaagtgggaaacaaataatctaagaatgaggagaaagcaagaagagtgacccccttgtgggcactccattggttttatggcgcctctactttctggagtttgtgtaaaacaaaaatattatggtctttgtgcacatttacatcaagctcagcctgggcggcacagccagatgcgagatgcgtctctgctgatctgagtctgcctgcagcatggacctgggtcttccctgaagcatctccagggctggagggacgactgccatgcaccgagggctcatccatccacagagcagggcagtgggaggagacgccatgacccccatcctcacggtcctgatctgtctcgggctgagtctgggcccccggacccacgtgcaggcagggcacctccccaagcccaccctctgggctgaaccaggctctgtgatcacccaggggagtcctgtgaccctcaggtgtcaggggggccaggagacccaggagtaccgtctatatagagaaaagaaaacagcaccctggattacacggatcccacaggagcttgtgaagaagggccagttccccatcccatccatcacctgggaacatgcagggcggtatcgctgttactatggtagcgacactgcaggccgctcagagagcagtgaccccctggagctggtggtgacaggagcctacatcaaacccaccctctcagcccagcccagccccgtggtgaactcaggagggaatgtaaccctccagtgtgactcacaggtggcatttgatggcttcattctgtgtaaggaaggagaagatgaacacccacaatgcctgaactcccagccccatgcccgtgggtcgtcccgcgccatcttctccgtgggccccgtgagcccgagtcgcaggtggtggtacaggtgctatgcttatgactcgaactctccctatgagtggtctctacccagtgatctcctggagctcctggtcctaggtgtttctaagaagccatcactctcagtgcagccaggtcctatcgtggcccctgaggagaccctgactctgcagtgtggctctgatgctggctacaacagatttgttctgtataaggacggggaacgtgacttccttcagctcgctggcgcacagccccaggctgggctctcccaggccaacttcaccctgggccctgtgagccgctcctacgggggccagtacagatgctacggtgcacacaacctctcctccgagtggtcggcccccagcgaccccctggacatcctgatcgcaggacagttctatgacagagtctccctctcggtgcagccgggccccacggtggcctcaggagagaacgtgaccctgctgtgtcagtcacagggatggatgcaaactttccttctgaccaaggagggggcagctgatgacccatggcgtctaagatcaacgtaccaatctcaaaaataccaggctgaattccccatgggtcctgtgacctcagcccatgcggggacctacaggtgctacggctcacagagctccaaaccctacctgctgactcaccccagtgaccccctggagctcgtggtctcaggaccgtctgggggccccagctccccgacaacaggccccacctccacatctggccctgaggaccagcccctcacccccaccgggtcggatccccagagtggtctgggaaggcacctgggggttgtgatcggcatcttggtggccgtcatcctactgctcctcctcctcctcctcctcttcctcatcctccgacatcgacgtcagggcaaacactggacatcgacccagagaaaggctgatttccaacatcctgcaggggctgtggggccagagcccacagacagaggcctgcagtggaggtccagcccagctgccgatgcccaggaagaaaacctctatgctgccgtgaagcacacacagcctgaggatggggtggagatggacactcggagcccacacgatgaagacccccaggcagtgacgtatgccgaggtgaaacactccagacctaggagagaaatggcctctcctccttccccactgtctggggaattcctggacacaaaggacagacaggcggaagaggacaggcagatggacactgaggctgctgcatctgaagccccccaggatgtgacctacgcccagctgcacagcttgacccttagacggaaggcaactgagcctcctccatcccaggaagggccctctccagctgtgcccagcatctacgccactctggccatccactagcccagggggggacgcagaccccacactccatggagtctggaatgcatgggagctgcccccccagtggacaccattggaccccacccagcctggatctaccccaggagactctgggaacttttaggggtcactcaattctgcagtataaataactaatgtctctacaattttgaaataaagcaacagacttctcaataatcaatgaagtagctgagaaaactaagtcagaaagtgcattaaactgaatcacaatgtaaatattacacatcaagcgatgaaactggaaaactacaagccacgaatgaatgaattaggaaagaaaaaaagtaggaaatgaatgatcttggctttcctataagaaatttagggcagggcacggtggctcacgcctgtaattccagcactttgggaggccgaggcgggcagatcacgagttcaggagatcgagaccatcttggccaacatggtgaaaccctgtctctcctaaaaatacaaaaattagctggatgtggtggcagtgcctgtaatcccagctatttgggaggctgaggcaggagaatcgcttgaaccagggagtcagaggtttcagtgagccaagatcgcaccactgctctccagcctggcgacagagggagactccatctcaaattaaaaaaaaaaaaaaaaaagaaagaaaaaaaaaaaaaaaaaa

SEQ ID NO: 3 - protein, 198 aa, Artificial Sequence
  Soluble two domain form of wild-type human ILT-2
MGHLPKPTLWAEPGSVITQGSPVTLRCQGGQETQEYRLYREKKTAPWITRIPQELVKKGQFPIPSITWEHAGRYRCYYGSDTAGRSESSDPLELVVTGAYIKPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELLVL

SEQ ID NO: 4 - DNA, 690 bp, Artificial Sequence
  DNA encoding soluble two domain form of wild-type human ILT-2
atggggcacctccccaagcccaccctctgggctgaaccaggctctgtgatcacccaggggagtcctgtgaccctcaggtgtcaggggggccaggagacccaggagtaccgtctatatagagaaaagaaaacagcaccctggattacacggatcccacaggagcttgtgaagaagggccagttccccatcccatccatcacctgggaacatgcagggcggtatcgctgttactatggtagcgacactgcaggccgctcagagagcagtgaccccctggagctggtggtgacaggagcctacatcaaacccaccctctcagcccagcccagccccgtggtgaactcaggagggaatgtaaccctccagtgtgactcacaggtggcatttgatggcttcattctgtgtaaggaaggagaagatgaacacccacaatgcctgaactcccagccccatgcccgtgggtcgtcccgcgccatcttctccgtgggccccgtgagcccgagtcgcaggtggtggtacaggtgctatgcttatgactcgaactctccctatgagtggtctctacccagtgatctcctggagctcctggtcctagcggccgcaggtggcggtactagtactgttgaaagttgtttagcaaaaccccatacagaaaattcatttactaacgtctggaaagacgacaaaact

SEQ ID NO: 5 - DNA, 615 bp, Artificial Sequence
  DNA encoding soluble two domain form of wild-type human ILT-2 including restriction enzyme recognition sequences
tatacatatgggtcatcttccaaaaccaactctctgggctgaaccaggctctgtgatcacccaggggagtcctgtgaccctcaggtgtcaggggggccaggagacccaggagtaccgtctatatagagaaaagaaaacagcaccctggattacacggatcccacaggagcttgtgaagaagggccagttccccatcccatccatcacctgggaacatgcagggcggtatcgctgttactatggtagcgacactgcaggccgctcagagagcagtgaccccctggagctggtggtgacaggagcctacatcaaacccaccctctcagcccagcccagccccgtggtgaactcaggagggaatgtaaccctccagtgtgactcacaggtggcatttgatggcttcattctgtgtaaggaaggagaagatgaacacccacaatgcctgaactcccagccccatgcccgtgggtcgtcccgcgccatcttctccgtgggccccgtgagcccgagccgcaggtggtggtacaggtgctatgcttatgactcgaactctccctatgagtggtctctacccagtgatctcctggagctcctggtcctataagcttgaattcc

SEQ ID NO: 6 - protein, 198 aa, Artificial Sequence
  Soluble high affinity mutated human ILT-2
MHLPKPTLWAEPGSVITLQSPVTLRCQGGQETQQYRLYREKKTAPWITRIPQELVKKGQFPIPSITWEHAGRYRCYYGSDTRQWSASSDPLELVVTGVYIKPTLSAQPSPVVNSGGNVTLQCDSQVAFDGFILCKEGEDDHPQCLNSQPHARGSSRAIFSVGPVSPSRRWRYRCYAYDSNSPYEWSLPSDLLELDVDG

SEQ ID NO: 7 - protein, 200 aa, Artificial Sequence
  Soluble high affinity mutated human ILT-2
MAGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYPIWTPYEWSLPSDLLELDVDG

SEQ ID NO: 8 - protein, 200 aa, Artificial Sequence
  Soluble high affinity mutated human ILT-2
MAGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYVAWAPYEWSLPSDLLELDVDG

SEQ ID NO: 9 - protein, 200 aa, Artificial Sequence
  Soluble high affinity mutated human ILT-2
MAGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYMNWAPYEWSLPSDLLELDVDG

SEQ ID NO: 10 - protein, 200 aa, Artificial Sequence
  Soluble high affinity mutated human ILT-2
MAGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYTDYSPYEWSLPSDLLELDVDG

SEQ ID NO: 11 - protein, 200 aa, Artificial Sequence
  Soluble high affinity mutated human ILT-2
MAGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYVDWFPYEWSLPSDLLELDVDG

SEQ ID NO: 12 - protein, 200 aa, Artificial Sequence
  Soluble high affinity mutated human ILT-2
MAGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYVSYWPYEWSLPSDLLELDVDG

SEQ ID NO: 13 - protein, 200 aa, Artificial Sequence
  Soluble high affinity mutated human ILT-2
MAGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYPAWTPYEWSLPSDLLELDVDG

SEQ ID NO: 14 - protein, 200 aa, Artificial Sequence
  Soluble high affinity mutated human ILT-2
MAQHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVDFDGFILCKEGEDEHPQCLNSQPHARGSSRAIFSVGPVSPSRRWWYRCYAYDSNSPYEWSLPSDLLELDVDG

SEQ ID NO: 15 - protein, 200 aa, Artificial Sequence
  Soluble high affinity mutated human ILT-2
MAQHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVSFDGFILCKEGEDEHPQCLNSQPHARGSSRAIFSVGPVSPSRRWWYRCYAYDSNSPYEWSLPSDLLELDVDG

SEQ ID NO: 16 - protein, 200 aa, Artificial Sequence
  Soluble high affinity mutated human ILT-2
MAQHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVTFDGFILCKEGEDEHPQCLNSQPHARGSSRAIFSVGPVSPSRRWWYRCYAYDSNSPYEWSLPSDLLELDVDG

SEQ ID NO: 17 - protein, 200 aa, Artificial Sequence
  Soluble high affinity mutated human ILT-2
MAQHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAYDGFILCKEGEDEHPQCLNSQPHARGSSRAIFSVGPVSPSRRWWYRCYAYDSNSPYEWSLPSDLLELDVDG

SEQ ID NO: 18 - protein, 200 aa, Artificial Sequence
  Soluble high affinity mutated human ILT-2
MAGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYMSWAPYEWSLPSDLLELDVDG

SEQ ID NO: 19 - protein, 200 aa, Artificial Sequence
  Soluble high affinity mutated human ILT-2
MAGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYMSWSPYEWSLPSDLLELDVDG

SEQ ID NO: 20 - protein, 200 aa, Artificial Sequence
  Soluble high affinity mutated human ILT-2
MAGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYPDFTPYEWSLPSDLLELDVDG

SEQ ID NO: 21 - protein, 200 aa, Artificial Sequence
  Soluble high affinity mutated human ILT-2
MAGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYPWFTPYEWSLPSDLLELDVDG

SEQ ID NO: 22 - protein, 200 aa, Artificial Sequence
  Soluble high affinity mutated human ILT-2
MAGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYGRWLPYEWSLPSDLLELDVDG

SEQ ID NO: 23 - protein, 200 aa, Artificial Sequence
  Soluble high affinity mutated human ILT-2
MAGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYVEWLPYEWSLPSDLLELDVDG

SEQ ID NO: 24 - protein, 198 aa, Artificial Sequence
  Soluble high affinity mutated human ILT-2
MHLPKPTLWAEPGSVITLQSPVTLRCQGGQETQQYRLYREKKTAPWITRIPQELVKKGQFPIPSITWEHAGRYRCYYGSDTRQWSASSDPLELVVTGVYIKPTLSAQPSPVVNSGGNVTLQCDSQVAFDGFILCKEGEDDHPQCLNSQPHARGSSRAIFSVGPVSPSRRWRYRCYAYPIWTPYEWSLPSDLLELDVDG

SEQ ID NO: 25 - protein, 198 aa, Artificial Sequence
  Soluble high affinity mutated human ILT-2
MHLPKPTLWAEPGSVITLQSPVTLRCQGGQETQQYRLYREKKTAPWITRIPQELVKKGQFPIPSITWEHAGRYRCYYGSDTRQWSASSDPLELVVTGVYIKPTLSAQPSPVVNSGGNVTLQCDSQVAFDGFILCKEGEDDHPQCLNSQPHARGSSRAIFSVGPVSPSRRWRYRCYAYVAWAPYEWSLPSDLLELDVDG

SEQ ID NO: 26 - protein, 198 aa, Artificial Sequence
  Soluble high affinity mutated human ILT-2
MHLPKPTLWAEPGSVITLQSPVTLRCQGGQETQQYRLYREKKTAPWITRIPQELVKKGQFPIPSITWEHAGRYRCYYGSDTRQWSASSDPLELVVTGVYIKPTLSAQPSPVVNSGGNVTLQCDSQVAFDGFILCKEGEDDHPQCLNSQPHARGSSRAIFSVGPVSPSRRWRYRCYAYMNWAPYEWSLPSDLLELDVDG

SEQ ID NO: 27 - protein, 198 aa, Artificial Sequence
  Soluble high affinity mutated human ILT-2
MHLPKPTLWAEPGSVITLQSPVTLRCQGGQETQQYRLYREKKTAPWITRIPQELVKKGQFPIPSITWEHAGRYRCYYGSDTRQWSASSDPLELVVTGVYIKPTLSAQPSPVVNSGGNVTLQCDSQVAFDGFILCKEGEDDHPQCLNSQPHARGSSRAIFSVGPVSPSRRWRYRCYAYPAWTPYEWSLPSDLLELDVDG

SEQ ID NO: 28 - protein, 198 aa, Artificial Sequence
  Soluble high affinity mutated human ILT-2
MHLPKPTLWAEPGSVITLQSPVTLRCQGGQETQQYRLYREKKTAPWITRIPQELVKKGQFPIPSITWEHAGRYRCYYGSDTRQWSASSDPLELVVTGVYIKPTLSAQPSPVVNSGGNVTLQCDSQVAFDGFILCKEGEDDHPQCLNSQPHARGSSRAIFSVGPVSPSRRWRYRCYAYMSWAPYEWSLPSDLLELDVDG

SEQ ID NO: 29 - protein, 198 aa, Artificial Sequence
  Soluble high affinity mutated human ILT-2
MHLPKPTLWAEPGSVITLQSPVTLRCQGGQETQQYRLYREKKTAPWITRIPQELVKKGQFPIPSITWEHAGRYRCYYGSDTRQWSASSDPLELVVTGVYIKPTLSAQPSPVVNSGGNVTLQCDSQVAFDGFILCKEGEDDHPQCLNSQPHARGSSRAIFSVGPVSPSRRWRYRCYAYMSWSPYEWSLPSDLLELDVDG

SEQ ID NO: 30 - protein, 200 aa, Artificial Sequence
  Soluble high affinity mutated human ILT-2
MAGHLPKPTLWAEPGSVITQGSPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYPIWTPYEWSLPSDLLELDVDG

SEQ ID NO: 31 - protein, 199 aa, Artificial Sequence
  Soluble high affinity mutated human ILT-2
MGHLPKPTLWAEPGSVITMDQPVTLRCQGGQETQEYRLYREKKTAPWITRIPQELVKKGQFPIPSITWEHAGRYRCYYGSDTAGRSESSDPLELVVTGVYIKPTLSAQPSPVVNSGGNVTLQCDSQVAFDGFILCKEGEDEHPQCLNSQPHARGSSRAIFSVGPVSPSRRWWYRCYAYPIWTPYEWSLPSDLLELDVDG

SEQ ID NO: 32 - protein, 200 aa, Artificial Sequence
  Soluble high affinity mutated human ILT-2
MAGHLPKPTLWAEPGSVITQGSPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYVAWAPYEWSLPSDLLELDVDG

SEQ ID NO: 33 - protein, 199 aa, Artificial Sequence
  Soluble high affinity mutated human ILT-2
MGHLPKPTLWAEPGSVITMDQPVTLRCQGGQETQEYRLYREKKTAPWITRIPQELVKKGQFPIPSITWEHAGRYRCYYGSDTAGRSESSDPLELVVTGVYIKPTLSAQPSPVVNSGGNVTLQCDSQVAFDGFILCKEGEDEHPQCLNSQPHARGSSRAIFSVGPVSPSRRWWYRCYAYVAWAPYEWSLPSDLLELDVDG

SEQ ID NO: 34 - protein, 200 aa, Artificial Sequence
  Soluble high affinity mutated human ILT-2
MAGHLPKPTLWAEPGSVITQGSPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYMNWAPYEWSLPSDLLELDVDG

SEQ ID NO: 35 - protein, 199 aa, Artificial Sequence
  Soluble high affinity mutated human ILT-2
MGHLPKPTLWAEPGSVITMDQPVTLRCQGGQETQEYRLYREKKTAPWITRIPQELVKKGQFPIPSITWEHAGRYRCYYGSDTAGRSESSDPLELVVTGVYIKPTLSAQPSPVVNSGGNVTLQCDSQVAFDGFILCKEGEDEHPQCLNSQPHARGSSRAIFSVGPVSPSRRWWYRCYAYMNWAPYEWSLPSDLLELDVDG

SEQ ID NO: 36 - protein, 200 aa, Artificial Sequence
  Soluble high affinity mutated human ILT-2
MAGHLPKPTLWAEPGSVITQGSPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYPAWTPYEWSLPSDLLELDVDG

SEQ ID NO: 37 - protein, 199 aa, Artificial Sequence
  Soluble high affinity mutated human ILT-2
MGHLPKPTLWAEPGSVITMDQPVTLRCQGGQETQEYRLYREKKTAPWITRIPQELVKKGQFPIPSITWEHAGRYRCYYGSDTAGRSESSDPLELVVTGVYIKPTLSAQPSPVVNSGGNVTLQCDSQVAFDGFILCKEGEDEHPQCLNSQPHARGSSRAIFSVGPVSPSRRWWYRCYAYPAWTPYEWSLPSDLLELDVDG

SEQ ID NO: 38 - protein, 200 aa, Artificial Sequence
  Soluble high affinity mutated human ILT-2
MAGHLPKPTLWAEPGSVITQGSPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYMSWAPYEWSLPSDLLELDVDG

SEQ ID NO: 39 - protein, 199 aa, Artificial Sequence
  Soluble high affinity mutated human ILT-2
MGHLPKPTLWAEPGSVITMDQPVTLRCQGGQETQEYRLYREKKTAPWITRIPQELVKKGQFPIPSITWEHAGRYRCYYGSDTAGRSESSDPLELVVTGVYIKPTLSAQPSPVVNSGGNVTLQCDSQVAFDGFILCKEGEDEHPQCLNSQPHARGSSRAIFSVGPVSPSRRWWYRCYAYMSWAPYEWSLPSDLLELDVDG

SEQ ID NO: 40 - protein, 200 aa, Artificial Sequence
  Soluble high affinity mutated human ILT-2
MAGHLPKPTLWAEPGSVITQGSPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYMSWSPYEWSLPSDLLELDVDG

SEQ ID NO: 41 - protein, 199 aa, Artificial Sequence
  Soluble high affinity mutated human ILT-2
MGHLPKPTLWAEPGSVITMDQPVTLRCQGGQETQEYRLYREKKTAPWITRIPQELVKKGQFPIPSITWEHAGRYRCYYGSDTAGRSESSDPLELVVTGVYIKPTLSAQPSPVVNSGGNVTLQCDSQVAFDGFILCKEGEDEHPQCLNSQPHARGSSRAIFSVGPVSPSRRWWYRCYAYMSWSPYEWSLPSDLLELDVDG

SEQ ID NO: 42 - protein, 198 aa, Artificial Sequence
  Soluble high affinity mutated human ILT-2
MHLPKPTLWAEPGSVITLQSPLTLRCQGGQETQQYRLYREKKTAPWITRIPQELVKKGQFPIPSITWEHAGRYRCYYGSDTRQWSASSDPLELVVTGVYIKPTLSAQPSPVVNSGGNVTLQCDSQVAFDGFILCKEGEDDHPQCLNSQPHARGSSRAIFSVGPVSPSRRWRYRCYAYDSNSPYEWSLPSDLLELDVDG

SEQ ID NO: 43 - protein, 200 aa, Artificial Sequence
  Soluble high affinity mutated human ILT-2
MAGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYMSYSPYEWSLPSDLLELDVDG

SEQ ID NO: 44 - protein, 200 aa, Artificial Sequence
  Soluble high affinity mutated human ILT-2
MAGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYPAYTPYEWSLPSDLLELDVDG

SEQ ID NO: 45 - protein, 200 aa, Artificial Sequence
  Soluble high affinity mutated human ILT-2
MAGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYMSNSPYEWSLPSDLLELDVDG

SEQ ID NO: 46 - protein, 200 aa, Artificial Sequence
  Soluble high affinity mutated human ILT-2
MAGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYDSWSPYEWSLPSDLLELDVDG

SEQ ID NO: 47 - protein, 200 aa, Artificial Sequence
  Soluble high affinity mutated human ILT-2
MAGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYMSNAPYEWSLPSDLLELDVDG

SEQ ID NO: 48 - protein, 200 aa, Artificial Sequence
  Soluble high affinity mutated human ILT-2
MAGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYDSWAPYEWSLPSDLLELDVDG

SEQ ID NO: 49 - protein, 198 aa, Artificial Sequence
  Soluble high affinity mutated human ILT-2
MHLPKPTLWAEPGSVITLQSPLTLRCQGGQETQQYRLYREKKTAPWITRIPQELVKKGQFPIPSITWEHAGRYRCYYGSDTRQWSASSDPLELVVTGVYIKPTLSAQPSPVVNSGGNVTLQCDSQVAFDGFILCKEGEDDHPQCLNSQPHARGSSRAIFSVGPVSPSRRWRYRCYAYMSWSPYEWSLPSDLLELDVDG

SEQ ID NO: 50 - protein, 198 aa, Artificial Sequence
  Soluble high affinity mutated human ILT-2
MHLPKPTLWAEPGSVITLQSPLTLRCQGGQETQQYRLYREKKTAPWITRIPQELVKKGQFPIPSITWEHAGRYRCYYGSDTRQWSASSDPLELVVTGVYIKPTLSAQPSPVVNSGGNVTLQCDSQVAFDGFILCKEGEDDHPQCLNSQPHARGSSRAIFSVGPVSPSRRWRYRCYAYMSWAPYEWSLPSDLLELDVDG

SEQ ID NO: 51 - protein, 200 aa, Artificial Sequence
  Soluble high affinity mutated human ILT-2
MAGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYPIWTPYEWSLGSDLLELDVDG

SEQ ID NO: 52 - protein, 200 aa, Artificial Sequence
  Soluble high affinity mutated human ILT-2
MAGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYPIWTPYEWSQMSDLLELDVDG

SEQ ID NO: 53 - protein, 200 aa, Artificial Sequence
  Soluble high affinity mutated human ILT-2
MAGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYPIWTPYEWTRGSDLLELDVDG

SEQ ID NO: 54 - protein, 200 aa, Artificial Sequence
  Soluble high affinity mutated human ILT-2
MAGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYPIWTPYEWSTSSDLLELDVDG

SEQ ID NO: 55 - protein, 200 aa, Artificial Sequence
  Soluble high affinity mutated human ILT-2
MAGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYPIWTPYEWSSGSDLLELDVDG

SEQ ID NO: 56 - protein, 200 aa, Artificial Sequence
  Soluble high affinity mutated human ILT-2
MAGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYPIWTPYEWTLGSDLLELDVDG

SEQ ID NO: 57 - protein, 200 aa, Artificial Sequence
  Soluble high affinity mutated human ILT-2
MAGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYDSNSPYEWTRGSDLLELDVDG

SEQ ID NO: 58 - protein, 200 aa, Artificial Sequence
  Soluble high affinity mutated human ILT-2
MAGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYDSNSPYEWSQMSDLLELDVDG

SEQ ID NO: 59 - protein, 200 aa, Artificial Sequence
  Soluble high affinity mutated human ILT-2
MAGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYDSNSPYEWSSGSDLLELDVDG

SEQ ID NO: 60 - protein, 199 aa, Artificial Sequence
  Soluble high affinity mutated human ILT-2
MGHLPKPTLWAEPGSVITMDQPVTLRCQGGQETQQYRLYREKKTAPWITRIPQELVKKGQFPIPSITWEHAGRYRCYYGSDTRQWSASSDPLELVVTGVYIKPTLSAQPSPVVNSGGNVTLQCDSQVAFDGFILCKEGEDDHPQCLNSQPHARGSSRAIFSVGPVSPSRRWRYRCYAYDSNSPYEWTRGSDLLELDVDG

SEQ ID NO: 61 - protein, 198 aa, Artificial Sequence
  Soluble high affinity mutated human ILT-2
MGHLPKPTLWAEPGSVITMGQPVTLRCQGGQETQEYRLYREKKTAPWITRIPQELVKKGQFPIPSITWEHAGRYRCYYGSDTAGRSESSDPLELVVTGAYIKPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELLVL

SEQ ID NO: 62 - protein, 198 aa, Artificial Sequence
  Soluble high affinity mutated human ILT-2
MGHLPKPTLWAEPGSVITMDQPVTLRCQGGQETQEYRLYREKKTAPWITRIPQELVKKGQFPIPSITWEHAGRYRCYYGSDTAGRSESSDPLELVVTGVYIKPTLSAQPSPVVNSGGNVTLQCDSQVAFDGFILCKEGEDEHPQCLNSQPHARGSSRAIFSVGPVSPGRRWWYRCYAYDSNSPYEWSLPSDLLELLVL

SEQ ID NO: 63 - protein, 198 aa, Artificial Sequence
  Soluble high affinity mutated human ILT-2
MGHLPKPTLWAEPGSVITLMQPVTLRCQGGQETQEYRLYREKKTAPWITRIPQELVKKGQFPIPSITWEHAGRYRCYYGSDTAGRSESSDPLELVVTGAYIKPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELLVL

SEQ ID NO: 64 - protein, 198 aa, Artificial Sequence
  Soluble high affinity mutated human ILT-2
MGHLPKPTLWAEPGSVITMQRPVTLRCQGGQETQEYRLYRERKTAPWITRIPQELVKKGQFPIPSITWEHAGRYRCYYGSDTAGRSESSDPLELVVTGAYIKPTLSAQPSPVVNSGGNVTLQCDSQVAFDGFILCKEGEDEHPQCLSSQPHARGSSRAIFSVGPVSPSRRWWYRCYAYDSNSPYEWSLPSDLLELLVL

SEQ ID NO: 65 - protein, 198 aa, Artificial Sequence
  Soluble high affinity mutated human ILT-2
MGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELLVL

SEQ ID NO: 66 - protein, 198 aa, Artificial Sequence
  Soluble high affinity mutated human ILT-2
MHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELDVDG

SEQ ID NO: 67 - protein, 199 aa, Artificial Sequence
  Soluble high affinity mutated human ILT-2
MHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELDVDGC

SEQ ID NO: 68 - protein, 198 aa, Artificial Sequence
  Soluble high affinity mutated human ILT-2
MGHLPKPTLWAEPGSVITLFQPVTLRCQGGQETQEYRLYREKKTAPWITRIPQELVKKGQFPIPSITWEHAGRYRCYYGSDTAGRSESSDPLELVVTGAYIKPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELLVL

SEQ ID NO: 69 - protein, 198 aa, Artificial Sequence
  Soluble high affinity mutated human ILT-2
MGHLPKPTLWAEPGSVITLSQPVTLRCQGGQETQEYRLYREKKTAPWITRIPQELVKKGQFPIPSITWEHAGRYRCYYGSDTAGRSESSDPLELVVTGVYIKPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELLVL

SEQ ID NO: 70 - protein, 198 aa, Artificial Sequence
  Soluble high affinity mutated human ILT-2
MGHLPKPTLQAEPGSVITQGSPVTLRCQGGQETQEYRLYREKKTAPWITRIPQELVKKGQFPIPSITWEHAGRYRCVIQRGTAGRSESSDPLELVVTGVYIKPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELLVL

SEQ ID NO: 71 - protein, 198 aa, Artificial Sequence
  Soluble high affinity mutated human ILT-2
MGHLPKPTLWAEPGSVITQGSPVTLRCQGGQETQEYRLYREKKTAPWITRIPQELVKKGQFPIPSITWEHAGRYRCYYGSQGGLRSESSDPLELVVTGVYIKPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELLVL

SEQ ID NO: 72 - protein, 198 aa, Artificial Sequence
  Soluble high affinity mutated human ILT-2
MGHLPKPTLWAEPGSVITQGSPVTLRCQGGQETQEYRLYREKKTAPWITRIPQELVKKGQFPIPSITWEHAGRYRCYYGTQTAGRSESSDPLELVVTGVYIKPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELLVL

SEQ ID NO: 73 - protein, 198 aa, Artificial Sequence
  Soluble high affinity mutated human ILT-2
MGHLPKPTLWAEPGSVITQGSPVTLRCQGGQETQEYRLYREKKTAPWITRIPQELVKKGQFPIPSITWEHAGRYRCMQYTLTAGRSESSDPLELVVTGVYIKPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELLVL

SEQ ID NO: 74 - protein, 198 aa, Artificial Sequence
  Soluble high affinity mutated human ILT-2
MGHLPKPTLWAEPGSVITQGSPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELLVL

SEQ ID NO: 75 - protein, 198 aa, Artificial Sequence
  Soluble high affinity mutated human ILT-2
MGHLPKPTLWAEPGSVITQGSPVTLRCQGGQETQEYRLYREKKTAPWITRIPQELVKKGQFPIPSITWEHAGRYRCYYGSQGGLRSESSDPLELVVTGAYIKPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELLVL

SEQ ID NO: 76 - protein, 198 aa, Artificial Sequence
  Soluble high affinity mutated human ILT-2
MGHLPKPTLWAEPGSVITQGSPVTLRCQGGQETQEYRLYREKKTAPWITRIPQELVKKGQFPIPSITWEHAGRYRCYYGTQTAGRSESSDPLELVVTGAYIKPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELLVL

SEQ ID NO: 77 - protein, 198 aa, Artificial Sequence
  Soluble high affinity mutated human ILT-2
MGHLPKPTLWAEPGSVITQGSPVTLRCQGGQETQEYRLYREKKTAPWITRIPQELVKKGQFPIPSITWEHAGRYRCMQYTLTAGRSESSDPLELVVTGAYIKPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELLVL

SEQ ID NO: 78 - protein, 198 aa, Artificial Sequence
  Soluble high affinity mutated human ILT-2
MGHLPKPTLWAEPGSVITMDQPVTLRCQGGQETQEYRLYREKKTAPWITRIPQELVKKGQFPIPSITWEHAGRYRCYYGSQGGLRSESSDPLELVVTGVYIKPTLSAQPSPVVNSGGNVTLQCDSQVAFDGFILCKEGEDEHPQCLNSQPHARGSSRAIFSVGPVSPGRRWWYRCYAYDSNSPYEWSLPSDLLELLVL

SEQ ID NO: 79 - protein, 198 aa, Artificial Sequence
  Soluble high affinity mutated human ILT-2
MGHLPKPTLWAEPGSVITMDQPVTLRCQGGQETQEYRLYREKKTAPWITRIPQELVKKGQFPIPSITWEHAGRYRCYYGTQTAGRSESSDPLELVVTGVYIKPTLSAQPSPVVNSGGNVTLQCDSQVAFDGFILCKEGEDEHPQCLNSQPHARGSSRAIFSVGPVSPGRRWWYRCYAYDSNSPYEWSLPSDLLELLVL

SEQ ID NO: 80 - protein, 198 aa, Artificial Sequence
  Soluble high affinity mutated human ILT-2
MGHLPKPTLWAEPGSVITMDQPVTLRCQGGQETQEYRLYREKKTAPWITRIPQELVKKGQFPIPSITWEHAGRYRCVIQRGTAGRSESSDPLELVVTGVYIKPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELLVL

SEQ ID NO: 81 - protein, 198 aa, Artificial Sequence
  Soluble high affinity mutated human ILT-2
MGHLPKPTLWAEPGSVITMDQPVTLRCQGGQETQEYRLYREKKTAPWITRIPQELVKKGQFPIPSITWEHAGRYRCMQYTLTAGRSESSDPLELVVTGVYIKPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELLVL

SEQ ID NO: 82 - protein, 198 aa, Artificial Sequence
  Soluble high affinity mutated human ILT-2
MGHLPKPTLWAEPGSVITQGSPVTLRCQGGQETQEYRLYREKKTAPWITRIPQELVKKGQFPIPSITWEHAGRYRCYYGSDTAGRSESSDPLELVVTGIYRQPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELLVL

SEQ ID NO: 83 - protein, 198 aa, Artificial Sequence
  Soluble high affinity mutated human ILT-2
MGHLPKPTLWAEPGSVITQGSPVTLRCQGGQETQEYRLYREKKTAPWITRIPQELVKKGQFPIPSITWEHAGRYRCYYGSDTAGRSESSDPLELVVTGIYLAPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELLVL

SEQ ID NO: 84 - protein, 198 aa, Artificial Sequence
  Soluble high affinity mutated human ILT-2
MGHLPKPTLWAEPGSVITQGSPVTLRCQGGQETQEYRLYREKKTAPWITRIPQELVKKGQFPIPSITWEHAGRYRCYYGSDTAGRSESSDPLELVVTGIYKAPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELLVL

SEQ ID NO: 85 - protein, 198 aa, Artificial Sequence
  Soluble high affinity mutated human ILT-2
MGHLPKPTLWAEPGSVITQGSPVTLRCQGGQETQEYRLYREKKTAPWITRIPQELVKKGQFPIPSITWEHAGRYRCYYGSDTAGRSESSDPLELVVTGIYQAPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELLVL

SEQ ID NO: 86 - protein, 198 aa, Artificial Sequence
  Soluble high affinity mutated human ILT-2
MGHLPKPTLWAEPGSVITQGAPVTLRCQGGQETQEYRLYREKKTAPWITRIPQELVKKGQFPIPSITWEHAGRYRCYYGSDTAGRSESSDPLELVVTGVYIKPTLSAQPSPVVNSGGNVTLQCDSQVAFDGFILCKEGEDEHPQCLNSQPHARGSSRAIFSVGPVSPGRRWWYRCYAYDSNSPYEWSLPSDLLELLVL

SEQ ID NO: 87 - protein, 198 aa, Artificial Sequence
  Soluble high affinity mutated human ILT-2
MGHLPKPTLWAEPGSVITQGRPVTLRCQGGQETQEYRLYREKKTAPWITRIPQELVKKGQFPIPSITWEHAGRYRCYYGSDTAGRSESSDPLELVVTGVYIKPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELLVL

SEQ ID NO: 88 - protein, 198 aa, Artificial Sequence
  Soluble high affinity mutated human ILT-2
MGHLPKPTLWAEPGSVITVGQPVTLRCQGGQETQEYRLYREKKTAPWITRIPQELVKKGQFPIPSITWEHAGRYRCYYGSDTAGRSESSDPLELVVTGVYIKPTLSAQPSPVVNSGGNVTLQCDSQVAFDGFILCKEGEDEHPQCLNSQPHARGSSRAIFSVGPVSPGRRWWYRCYAYDSNSPYEWSLPSDLLELLVL

SEQ ID NO: 89 - protein, 198 aa, Artificial Sequence
  Soluble high affinity mutated human ILT-2
MGHLPKPTLWAEPGSVITLGQPVTLRCQGGQETQEYRLYREKKTAPWITRIPQELVKKGQFPIPSITWEHAGRYRCYYGSDTAGRSESSDPLELVVTGVYIKPTLSAQPSPVVNSGGNVTLQCDSQVAFDGFILCKEGEDEHPQCLNSQPHARGSSRAIFSVGPVSPGRRWWYRCYAYDSNSPYEWSLPSDLLELLVL

SEQ ID NO: 90 - protein, 198 aa, Artificial Sequence
  Soluble high affinity mutated human ILT-2
MGHLPKPTLWAEPGSVITQGSPVTLRCQGGQETQEYRLYREKKTAPWITRIPQELVKKGQFPIPSITWEHAGRYRCYYGSDTAGRSESSDPLELVVTGVYIKPTLSAQPSPVVNSGGNVTLQCDSQVAFDGFILCKEGEDEHPQCLNSQPHARGSSRAIFSVGPVSPGRRWWYRCYAYDSNSPYEWSLPSDLLELLVL

SEQ ID NO: 91 - protein, 198 aa, Artificial Sequence
  Soluble high affinity mutated human ILT-2
MGHLPKPTLWAEPGSVITQGSPVTLRCQGGQETQEYRLYREKKTAPWITRIPQELVKKGQFPIPSVTWEHAGRYRCYYGSDTAGRSESSDPLELVVTGVYIKPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELLVL

SEQ ID NO: 92 - protein, 198 aa, Artificial Sequence
  Soluble high affinity mutated human ILT-2
MGHLPKPTLWAEPGSVITQGSPVTLRCQGGQETQEYRLYREKKTAPWITRIPQELVKKGQFPIPSITWEHAGRYRCVGWAVTAGRSESSDPLELVVTGVYIKPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELLVL

SEQ ID NO: 93 - protein, 198 aa, Artificial Sequence
  Soluble high affinity mutated human ILT-2
MGHLPKPTLWAEPGSVITQGSPVTLRCQGGQETQEYRLYREKKTAPWITRIPQELVKKGQFPIPSITWEHAGRYRCIGRSQTAGRSESSDPLELVVTGVYIKPTLSAQPSPVVNSGGNVTLQCDSQVAFDGFILCKEGEDEHPQCLNSQPHARGSSRAIFSVGPVSPSRXWWYRCYAYDSNSPYEWSLPSDLLELLVL

SEQ ID NO: 94 - protein, 198 aa, Artificial Sequence
  Soluble high affinity mutated human ILT-2
MGHLPKPTLWAEPGSVITQGSPVTLRCQGGQETQEYRLYREKKTAPWITRIPQELVKKGQFPIPSITWEHAGRYRCYYGGQEGARSESSDPLELVVTGVYIKPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELLVL

SEQ ID NO: 95 - protein, 198 aa, Artificial Sequence
  Soluble high affinity mutated human ILT-2
MGHLPKPTLWAEPGSVITQGSPVTLRCQGGQETQEYRLYREKKTAPWITRIPQELVKKGQFPIPSITWEHAGRYRCQGVSQTAGRSESSDPLELVVTGVYIKPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELLVL

SEQ ID NO: 96 - protein, 198 aa, Artificial Sequence
  Soluble high affinity mutated human ILT-2
MGHLPKPTLWAEPGSVITQGSPVTLRCQGGQETQEYRLYREKKTAPQITRIPQELVKKGQFPIPSITWEHAGRYRCYYGSDTAGRSESSDPLELVVTGVYVAPTLSAQPSPVVNSGGNVTLQCDSQVAFDGFILCKEGEDEHPQCLNSQPHARGSSRAIFSVGPVSPGRRWWYRCYAYDSNSPYEWSLPSDLLELLVL

SEQ ID NO: 97 - protein, 198 aa, Artificial Sequence
  Soluble high affinity mutated human ILT-2
MGHLPKPTLWAEPGSVITQGSPVTLRCQGGQETQEYRLYREKKTAPWITRIPQELVKKGQFPIPSITWEHAGRYRCYYGSDTAGRSESSDPLELVVTGIYKAPTLSAQPSPVVNSGGNVTLQCDSQVAFDGFILCKEGEDEHPQCLNSQPHARGSSRAIFSVGPVSPGRRWWYRCYAYDSNSPYEWSLPSDLLELLVL

SEQ ID NO: 98 - protein, 198 aa, Artificial Sequence
  Soluble high affinity mutated human ILT-2
MGHLPKPTLWAEPGSVITQGSPVTLRCQGGQETQEYRLYREKKTAPWITRIPQELVKKGQFPIPSITWEHAGRYRCYYGSDTAGRSESSDPLELVVTGIYQRPTLSAQPSPVVNSGGNVTLQCDSQVAFDGFILCKEGEDEHPQCLNSQPHARGSSRAIFSVGPVSPGRRWWYRCYAYDSNSPYEWSLPSDLLELLVL

SEQ ID NO: 99 - protein, 198 aa, Artificial Sequence
  Soluble high affinity mutated human ILT-2
MGHLPKPTLWAEPGSVITQGSPVTLRCQGGQETQEYRLYREKKTAPWITRIPQELVKKGQFPIPSITWEHAGRYRCYYGSDTAGRSESSDPLELVVTGIYQAPTLSAQPSPVVNSGGNVTLQCDSQVAFDGFILCKEGEDEHPQCLNSQPHARGSSRAIFSVGPVSPGRRWWYRCYAYDSNSPYEWSLPSDLLELLVL

SEQ ID NO: 100 - protein, 198 aa, Artificial Sequence
  Soluble high affinity mutated human ILT-2
MGHLPKPTLWAEPGSVITQGSPVTLRCQGGQETQEYRLYREKKTAPWITRIPQELVKKGQFPIPSITWEHAGRYRCYYGSDTAGRSESSDPLELVVTGIYLQPTLSAQPSPVVNSGGNVTLQCDSQVAFDGFILCKEGEDEHPQCLNSQPHARGSSRAIFSVGPVSPGRRWWYRCYAYDSNSPYEWSLPSDLLELLVL

SEQ ID NO: 101 - protein, 198 aa, Artificial Sequence
  Soluble high affinity mutated human ILT-2
MGHLPKPTLWAEPGSVITQGSPVTLRCQGGQETQEYRLYREKKTAPWITRIPQELVKKGQFPIPSITWEHAGRYRCYYGSDTAGRSESSDPLELVVTGIYKQPTLSAQPSPVVNSGGNVTLQCDSQVAFDGFILCKEGEDEHPQCLNSQPHARGSSRAIFSVGPVSPGRRWWYRCYAYDSNSPYEWSLPSDLLELLVL

SEQ ID NO: 102 - protein, 198 aa, Artificial Sequence
  Soluble high affinity mutated human ILT-2
MGHLPKPTLWAEPGSVITQGSPVTLRCQGGQETQEYRLYREKKTAPWITRIPQELVKKGQFPIPSITWEHAGRYRCYYGSDTAGRSESSDPLELVVTGIYQKPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELLVL

SEQ ID NO: 103 - protein, 198 aa, Artificial Sequence
  Soluble high affinity mutated human ILT-2
MGHLPKPTLWAEPGSVITMDQPVTLRCQGGQETQEYRLYREKKTAPWITRIPQELVKKGQFPIPSITWEHAGRYRCYYGSDTAGRSESSDPLELVVTGIYLAPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELLVL

SEQ ID NO: 104 - protein, 198 aa, Artificial Sequence
  Soluble high affinity mutated human ILT-2
MGHLPKPTLWAEPGSVITLGQPVTLRCQGGQETQEYRLYREKKTAPWITRIPQELVKKGQFPIPSITWEHAGRYRCYYGSDTAGRSESSDPLELVVTGIYLAPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELLVL

SEQ ID NO: 105 - protein, 198 aa, Artificial Sequence
  Soluble high affinity mutated human ILT-2
MGHLPKPTLWAEPGSVITMDQPVTLRCQGGQETQEYRLYREKKTAPWITRIPQELVKKGQFPIPSITWEHAGRYRCYYGSDTSQWSASSDPLELVVTGIYLAPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELLVL

SEQ ID NO: 106 - protein, 198 aa, Artificial Sequence
  Soluble high affinity mutated human ILT-2
MGHLPKPTLWAEPGSVITLGQPVTLRCQGGQETQEYRLYREKKTAPWITRIPQELVKKGQFPIPSITWEHAGRYRCYYGSDTSQWSASSDPLELVVTGIYLAPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELLVL

SEQ ID NO: 107 - protein, 198 aa, Artificial Sequence
  Soluble high affinity mutated human ILT-2
MGHLPKPTLWAEPGSVITMDQPVTLRCQGGQETQEYRLYREKKTAPWITRIPQELVKKGQFPIPSITWEHAGRYRCIGRSQTSQWSASSDPLELVVTGIYLAPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELLVL

SEQ ID NO: 108 - protein, 198 aa, Artificial Sequence
  Soluble high affinity mutated human ILT-2
MGHLPKPTLWAEPGSVITLGQPVTLRCQGGQETQEYRLYREKKTAPWITRIPQELVKKGQFPIPSITWEHAGRYRCIGRSQTSQWSASSDPLELVVTGIYLAPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELLVL

SEQ ID NO: 109 - protein, 198 aa, Artificial Sequence; identical to SEQ ID NO: 66
  Soluble high affinity mutated human ILT-2

SEQ ID NO: 110 - protein, 198 aa, Artificial Sequence
  Soluble high affinity mutated human ILT-2
MHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWRYRCYAYDSNSPYEWSLPSDLLELDVDG

SEQ ID NO: 111 - protein, 198 aa, Artificial Sequence
  Soluble high affinity mutated human ILT-2
MHLPKPTLWAEPGSVITMDQPVTLRCQGGQETQEYRLYREKKTAPWITLIPQELVKKGQFPIPSITWEHAGRYRCYYGSDTSQWSASSDPLELVVTGVYIKPTLSAQPSPVVNSGGNVTLQCDSQVAFDGFILCKEGEDGHPQCLNSQPHARGSSRAIFSVGPVSPSRRWWYWCYAYDSNSPYEWSLPSDLLELDVDG

SEQ ID NO: 112 - protein, 198 aa, Artificial Sequence
  Soluble high affinity mutated human ILT-2
MHLPKPTLWAEPGSVITMDQPVTLRCQGGQETQEYRLYRERKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELDVDG

SEQ ID NO: 113 - protein, 198 aa, Artificial Sequence
  Soluble high affinity mutated human ILT-2
MHLPKPTLWAEPGSVITLGQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELDVDG

SEQ ID NO: 114 - protein, 198 aa, Artificial Sequence
  Soluble high affinity mutated human ILT-2
MHLPKPTLWAEPGSVITLGSPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELDVDG

SEQ ID NO: 115 - protein, 198 aa, Artificial Sequence
  Soluble high affinity mutated human ILT-2
MHLPKPTLWAEPGSVITLRSPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELDVDG

SEQ ID NO: 116 - protein, 198 aa, Artificial Sequence
  Soluble high affinity mutated human ILT-2
MHLPKPTLWAEPGSVITLQSPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELDVDG

SEQ ID NO: 117 - protein, 198 aa, Artificial Sequence
  Soluble high affinity mutated human ILT-2
MHLPKPTLWAEPGSVITLESPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELDVDG

SEQ ID NO: 118 - protein, 198 aa, Artificial Sequence
  Soluble high affinity mutated human ILT-2
MHLPKPTLWAEPGSVITMDQPVTLRCQGGQETQEYRLYREKKTAPWITRIPQELVKKGQFPIPSITWEHAGRYRCYYGTQTAGRSESSDPLELVVTGVYIKPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELDVDG

SEQ ID NO: 119 - protein, 198 aa, Artificial Sequence
  Soluble high affinity mutated human ILT-2
MHLPKPTLWAEPGSVITMDQPVTLRCQGGQETQEYRLYREKKTAPWITRIPQELVKKGQFPIPSITWEHAGRYRCMQYTLTAGRSESSDPLELVVTGVYIKPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELDVDG

SEQ ID NO: 120 - protein, 198 aa, Artificial Sequence
  Soluble high affinity mutated human ILT-2
MHLPKPTLWAEPGSVITMDQPVTLRCQGGQETQEYRLYREKKTAPWITRIPQELVKKGQFPIPSITWEHAGRYRCVIQRGTAGRSESSDPLELVVTGVYIKPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELDVDG

SEQ ID NO: 121 - protein, 198 aa, Artificial Sequence
  Soluble high affinity mutated human ILT-2
MHLPKPTLWAEPGSVITMDQPVTLRCQGGQETQEYRLYREKKTAPWITRIPQELVKKGQFPIPSITWEHAGRYRCYYGGQEGARSESSDPLELVVTGVYIKPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELDVDG

SEQ ID NO: 122 - protein, 198 aa, Artificial Sequence
  Soluble high affinity mutated human ILT-2
MHLPKPTLWAEPGSVITMDQPVTLRCQGGQETQEYRLYREKKTAPWITRIPQELVKKGQFPIPSITWEHAGRYRCYYGTQTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELDVDG

SEQ ID NO: 123 - protein, 199 aa, Artificial Sequence
  Soluble high affinity mutated human ILT-2
MGHLPKPTLWAEPGSVITMDQPVTLRCQGGQETQQYRLYREKKTAPWITRIPQELVKKGQFPIPSITWEHAGRYRCYYGSDTRQWSASSDPLELVVTGVYIKPTLSAQPSPVVNSGGNVTLQCDSQVAFDGFILCKEGEDDHPQCLNSQPHARGSSRAIFSVGPVSPSRRWRYRCYAYDSNSPYEWSLPSDLLELDVDG

SEQ ID NO: 124 - protein, 198 aa, Artificial Sequence
  Soluble high affinity mutated human ILT-2
MHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCDNSQPHARGSSRAIFSVGPVSPSRRWWYRCYAYDSNSPYEWSLPSDLLELDVDG

SEQ ID NO: 125 - protein, 198 aa, Artificial Sequence
  Soluble high affinity mutated human ILT-2
MHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAEFSVGPVSPSRRWWYRCYAYDSNSPYEWSLPSDLLELDVDG

SEQ ID NO: 126 - protein, 198 aa, Artificial Sequence
  Soluble high affinity mutated human ILT-2
MHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYDSNSPYEWSDPSDLLELDVDG

SEQ ID NO: 127 - protein, 198 aa, Artificial Sequence
  Soluble high affinity mutated human ILT-2
MHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILSKEGEDEHPQSLNSQPHARGSSRAIFSVGPVSPSRRWWYRCYAYDSNSPYEWSLPSDLLELDVDG

SEQ ID NO: 128 - protein, 199 aa, Artificial Sequence
  Soluble high affinity mutated human ILT-2
MGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSPWVADGFILCKEGEDEHPQCLNSQPHARGSSRAIFSVGPVSPSRRWWYRCYAYMSWSPYEWSLPSDLLELDVDG

SEQ ID NO: 129 - protein, 199 aa, Artificial Sequence
  Soluble high affinity mutated human ILT-2
MGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSPFLTDGFILCKEGEDEHPQCLNSQPHARGSSRAIFSVGPVSPSRRWWYRCYAYMSWSPYEWSLPSDLLELDVDG

SEQ ID NO: 130 - protein, 199 aa, Artificial Sequence
  Soluble high affinity mutated human ILT-2
MGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSPWLADGFILCKEGEDEHPQCLNSQPHARGSSRAIFSVGPVSPSRRWWYRCYAYMSWSPYEWSLPSDLLELDVDG

SEQ ID NO: 131 - protein, 199 aa, Artificial Sequence
  Soluble high affinity mutated human ILT-2
MGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSPFQADGFILCKEGEDEHPQCLNSQPHARGSSRAIFSVGPVSPSRRWWYRCYAYMSWSPYEWSLPSDLLELDVDG

SEQ ID NO: 132 - protein, 199 aa, Artificial Sequence
  Soluble high affinity mutated human ILT-2
MGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSPFIAEGFILCKEGEDEHPQCLNSQPHARGSSRAIFSVGPVSPSRRWWYRCYAYMSWSPYEWSLPSDLLELDVDG

SEQ ID NO: 133 - protein, 199 aa, Artificial Sequence
  Soluble high affinity mutated human ILT-2
MGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSPFLSDGFILCKEGEDEHPQCLNSQPHARGSSRAIFSVGPVSPSRKWWYRCYAYMSWSPYEWSLPSDLLELDVDG

SEQ ID NO: 134 - protein, 199 aa, Artificial Sequence
  Soluble high affinity mutated human ILT-2
MGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSPFVSDGFILCKEGEDEHPQCLNSQPHARGSSRAIFSVGPVSPSRRWWYRCYAYMSWSPYEWSLPSDLLELDVDG

SEQ ID NO: 135 - protein, 199 aa, Artificial Sequence
  Soluble high affinity mutated human ILT-2
MGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSPFIADGFILCKEGEDEHPQCLNSQPHARGSSRAIFSVGPVSPSRRWWYRCYAYMSWSPYEWSLPSDLLELDVDG

SEQ ID NO: 136 - protein, 199 aa, Artificial Sequence
  Soluble high affinity mutated human ILT-2
MGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSMFLVDGFILCKEGEDEHPQCLNSQPHARGSSRAIFSVGPVSPSRRWWYRCYAYMSWSPYEWSLPSDLLELDVDG

SEQ ID NO: 137 - DNA, 3111 bp, Artificial Sequence
  pGMT7-based vector
gatctcgatcccgcgaaattaatacgactcactatagggagaccacaacggtttccctctagaaataattttgtttaactttaagaaggagatatacatatgggatccatggtaagcttgaattccgatccggctgctaacaaagcccgaaaggaagctgagttggctgctgccaccgctgagcaataactagcataaccccttggggcctctaaacgggtcttgaggggttttttgctgaaaggaggaactatatccggataattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcag

SEQ ID NO: 138 - protein, 9 aa, Homo sapiens
YLSGANLNL

SEQ ID NO: 139 - protein, 226 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 140 - protein, 226 aa, Artificial Sequence
  Mutated human IgG1 Fc monomer
DKTHTCPPCPAPPVAGPSVFLFPPKPKDQLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 141 - protein, 227 aa, Artificial Sequence
  Mutated human IgG1 Fc monomer
DKTHTSPPSPAPELLGGSSVFLFPPKRKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 142 - protein, 226 aa, Artificial Sequence
  Mutated human IgG1 Fc monomer
DKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 143 - protein, 176 aa, Artificial Sequence
  Mutated human IgG1 Fc monomer
VECPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDS

SEQ ID NO: 144 - protein, 430 aa, Artificial Sequence
  High affinity ILT-like Fc fusion polypeptide monomer
HLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYMSWSPYEWSLPSDLLELDVDGRMDPSRSDKTHTCPPCPAPPVAGPSVFLFPPKPKDQLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 145 - protein, 427 aa, Artificial Sequence
  High affinity ILT-like Fc fusion polypeptide monomer
HLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYMSWSPYEWSLPSDLLELDVDGPSRSDKTHTCPPCPAPPVAGPSVFLFPPKPKDQLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 146 - protein, 431 aa, Artificial Sequence
  High affinity ILT-like Fc fusion polypeptide monomer
HLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYMSWSPYEWSLPSDLLELDVDGRMDPSRSDKTHTSPPSPAPELLGGSSVFLFPPKRKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 147 - protein, 428 aa, Artificial Sequence
  High affinity ILT-like Fc fusion polypeptide monomer
HLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYMSWSPYEWSLPSDLLELDVDGPSRSDKTHTSPPSPAPELLGGSSVFLFPPKRKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 148 - protein, 430 aa, Artificial Sequence
  High affinity ILT-like Fc fusion polypeptide monomer
HLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYMSWSPYEWSLPSDLLELDVDGRMDPSRSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 149 - protein, 427 aa, Artificial Sequence
  High affinity ILT-like Fc fusion polypeptide monomer
HLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYMSWSPYEWSLPSDLLELDVDGPSRSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 150 - protein, 427 aa, Artificial Sequence
  High affinity ILT-like Fc fusion polypeptide monomer
HLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYMSWSPYEWSLPSDLLELDVDGRMDPSRSVECPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEGLHNHYTQKSLSLSPGK

SEQ ID NO: 151 - protein, 424 aa, Artificial Sequence
  High affinity ILT-like Fc fusion polypeptide monomer
HLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYMSWSPYEWSLPSDLLELDVDGPSRSVECPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEGLHNHYTQKSLSLSPGK

SEQ ID NO: 152 - protein, 430 aa, Artificial Sequence
  High affinity ILT-like Fc fusion polypeptide monomer
HLPKPTLWAEPGSVITMDQPVTLRCQGGQETQQYRLYREKKTAPWITRIPQELVKKGQFPIPSITWEHAGRYRCYYGSDTRQWSASSDPLELVVTGVYIKPTLSAQPSPVVNSGGNVTLQCDSQVAFDGFILCKEGEDDHPQCLNSQPHARGSSRAIFSVGPVSPSRRWRYRCYAYDSNSPYEWSLPSDLLELDVDGRMDPSRSDKTHTCPPCPAPPVAGPSVFLFPPKPKDQLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 153 - protein, 427 aa, Artificial Sequence
  High affinity ILT-like Fc fusion polypeptide monomer
HLPKPTLWAEPGSVITMDQPVTLRCQGGQETQQYRLYREKKTAPWITRIPQELVKKGQFPIPSITWEHAGRYRCYYGSDTRQWSASSDPLELVVTGVYIKPTLSAQPSPVVNSGGNVTLQCDSQVAFDGFILCKEGEDDHPQCLNSQPHARGSSRAIFSVGPVSPSRRWRYRCYAYDSNSPYEWSLPSDLLELDVDGPSRSDKTHTCPPCPAPPVAGPSVFLFPPKPKDQLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 154 - protein, 431 aa, Artificial Sequence
  High affinity ILT-like Fc fusion polypeptide monomer
HLPKPTLWAEPGSVITMDQPVTLRCQGGQETQQYRLYREKKTAPWITRIPQELVKKGQFPIPSITWEHAGRYRCYYGSDTRQWSASSDPLELVVTGVYIKPTLSAQPSPVVNSGGNVTLQCDSQVAFDGFILCKEGEDDHPQCLNSQPHARGSSRAIFSVGPVSPSRRWRYRCYAYDSNSPYEWSLPSDLLELDVDGRMDPSRSDKTHTSPPSPAPELLGGSSVFLFPPKRKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 155 - protein, 428 aa, Artificial Sequence
  High affinity ILT-like Fc fusion polypeptide monomer
HLPKPTLWAEPGSVITMDQPVTLRCQGGQETQQYRLYREKKTAPWITRIPQELVKKGQFPIPSITWEHAGRYRCYYGSDTRQWSASSDPLELVVTGVYIKPTLSAQPSPVVNSGGNVTLQCDSQVAFDGFILCKEGEDDHPQCLNSQPHARGSSRAIFSVGPVSPSRRWRYRCYAYDSNSPYEWSLPSDLLELDVDGPSRSDKTHTSPPSPAPELLGGSSVFLFPPKRKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 156 - protein, 430 aa, Artificial Sequence
  High affinity ILT-like Fc fusion polypeptide monomer
HLPKPTLWAEPGSVITMDQPVTLRCQGGQETQQYRLYREKKTAPWITRIPQELVKKGQFPIPSITWEHAGRYRCYYGSDTRQWSASSDPLELVVTGVYIKPTLSAQPSPVVNSGGNVTLQCDSQVAFDGFILCKEGEDDHPQCLNSQPHARGSSRAIFSVGPVSPSRRWRYRCYAYDSNSPYEWSLPSDLLELDVDGRMDPSRSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 157 - protein, 427 aa, Artificial Sequence
  High affinity ILT-like Fc fusion polypeptide monomer
HLPKPTLWAEPGSVITMDQPVTLRCQGGQETQQYRLYREKKTAPWITRIPQELVKKGQFPIPSITWEHAGRYRCYYGSDTRQWSASSDPLELVVTGVYIKPTLSAQPSPVVNSGGNVTLQCDSQVAFDGFILCKEGEDDHPQCLNSQPHARGSSRAIFSVGPVSPSRRWRYRCYAYDSNSPYEWSLPSDLLELDVDGPSRSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 158 - protein, 427 aa, Artificial Sequence
  High affinity ILT-like Fc fusion polypeptide monomer
HLPKPTLWAEPGSVITMDQPVTLRCQGGQETQQYRLYREKKTAPWITRIPQELVKKGQFPIPSITWEHAGRYRCYYGSDTRQWSASSDPLELVVTGVYIKPTLSAQPSPVVNSGGNVTLQCDSQVAFDGFILCKEGEDDHPQCLNSQPHARGSSRAIFSVGPVSPSRRWRYRCYAYDSNSPYEWSLPSDLLELDVDGRMDPSRSVECPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEGLHNHYTQKSLSLSPGK

SEQ ID NO: 159 - protein, 424 aa, Artificial Sequence
  High affinity ILT-like Fc fusion polypeptide monomer
HLPKPTLWAEPGSVITMDQPVTLRCQGGQETQQYRLYREKKTAPWITRIPQELVKKGQFPIPSITWEHAGRYRCYYGSDTRQWSASSDPLELVVTGVYIKPTLSAQPSPVVNSGGNVTLQCDSQVAFDGFILCKEGEDDHPQCLNSQPHARGSSRAIFSVGPVSPSRRWRYRCYAYDSNSPYEWSLPSDLLELDVDGPSRSVECPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEGLHNHYTQKSLSLSPGK

SEQ ID NO: 160 - protein, 430 aa, Artificial Sequence
  High affinity ILT-like Fc fusion polypeptide monomer
HLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSPFQADGFILCKEGEDEHPQCLNSQPHARGSSRAIFSVGPVSPSRRWWYRCYAYMSWSPYEWSLPSDLLELDVDGRMDPSRSDKTHTCPPCPAPPVAGPSVFLFPPKPKDQLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 161 - protein, 427 aa, Artificial Sequence
  High affinity ILT-like Fc fusion polypeptide monomer
HLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSPFQADGFILCKEGEDEHPQCLNSQPHARGSSRAIFSVGPVSPSRRWWYRCYAYMSWSPYEWSLPSDLLELDVDGPSRSDKTHTCPPCPAPPVAGPSVFLFPPKPKDQLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 162 - protein, 431 aa, Artificial Sequence
  High affinity ILT-like Fc fusion polypeptide monomer
HLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSPFQADGFILCKEGEDEHPQCLNSQPHARGSSRAIFSVGPVSPSRRWWYRCYAYMSWSPYEWSLPSDLLELDVDGRMDPSRSDKTHTSPPSPAPELLGGSSVFLFPPKRKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 163 - protein, 428 aa, Artificial Sequence
  High affinity ILT-like Fc fusion polypeptide monomer
HLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSPFQADGFILCKEGEDEHPQCLNSQPHARGSSRAIFSVGPVSPSRRWWYRCYAYMSWSPYEWSLPSDLLELDVDGPSRSDKTHTSPPSPAPELLGGSSVFLFPPKRKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 164 - protein, 430 aa, Artificial Sequence
  High affinity ILT-like Fc fusion polypeptide monomer
HLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSPFQADGFILCKEGEDEHPQCLNSQPHARGSSRAIFSVGPVSPSRRWWYRCYAYMSWSPYEWSLPSDLLELDVDGRMDPSRSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 165 - protein, 427 aa, Artificial Sequence
  High affinity ILT-like Fc fusion polypeptide monomer
HLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSPFQADGFILCKEGEDEHPQCLNSQPHARGSSRAIFSVGPVSPSRRWWYRCYAYMSWSPYEWSLPSDLLELDVDGPSRSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 166 - protein, 427 aa, Artificial Sequence
  High affinity ILT-like Fc fusion polypeptide monomer
HLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSPFQADGFILCKEGEDEHPQCLNSQPHARGSSRAIFSVGPVSPSRRWWYRCYAYMSWSPYEWSLPSDLLELDVDGRMDPSRSVECPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEGLHNHYTQKSLSLSPGK

SEQ ID NO: 167 - protein, 424 aa, Artificial Sequence
  High affinity ILT-like Fc fusion polypeptide monomer
HLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSPFQADGFILCKEGEDEHPQCLNSQPHARGSSRAIFSVGPVSPSRRWWYRCYAYMSWSPYEWSLPSDLLELDVDGPSRSVECPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEGLHNHYTQKSLSLSPGK

SEQ ID NO: 168 - protein, 197 aa, Artificial Sequence
  Soluble high affinity mutated human ILT-2
HLPKPTLWAEPGSVITMDQPVTLRCQGGQETQQYRLYREKKTAPWITRIPQELVKKGQFPIPSITWEHAGRYRCYYGSDTRQWSASSDPLELVVTGVYIKPTLSAQPSPVVNSGGNVTLQCDSPFQADGFILCKEGEDDHPQCLNSQPHARGSSRAIFSVGPVSPSRRWRYRCYAYDSNSPYEWSLPSDLLELDVDG

SEQ ID NO: 169 - protein, 427 aa, Artificial Sequence
  High affinity ILT-like Fc fusion polypeptide monomer
HLPKPTLWAEPGSVITMDQPVTLRCQGGQETQQYRLYREKKTAPWITRIPQELVKKGQFPIPSITWEHAGRYRCYYGSDTRQWSASSDPLELVVTGVYIKPTLSAQPSPVVNSGGNVTLQCDSPFQADGFILCKEGEDDHPQCLNSQPHARGSSRAIFSVGPVSPSRRWRYRCYAYDSNSPYEWSLPSDLLELDVDGRMDPSRSVECPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEGLHNHYTQKSLSLSPGK

SEQ ID NO: 170 - protein, 200 aa, Artificial Sequence
  Soluble high affinity mutated human ILT-2
MAGHLPKPTLWAEPGSVITMDQPVTLRCQGGQETQEYRLYREKKTAPWITRIPQELVKKGQFPIPSITWEHAGRYRCYYGSDTSQWSASSDPLELVVTGVYIKPTLSAQPSPVVNSGGNVTLQCDSQVAFDGFILCKEGEDEHPQCLNSQPHARGSSRAIFSVGPVSPSRRWWYRCYAYVNFWPYEWSLPSDLLELDVDG

(19) United States
(12) Patent Application Publication
(10) Pub. No.: US 2010/0135997 A1
(43) Pub. Date: Jun. 3, 2010

(54) POLYPEPTIDE MONOMERS AND DIMERS CONTAINING MUTATED ILT

(75) Inventors: Bent Karsten Jakobsen, Oxfordshire (GB); Yi Li, Oxfordshire (GB); Ruth Karen Moysey, Oxfordshire (GB)

Correspondence Address: CLARK & ELBING LLP, 101 FEDERAL STREET, BOSTON, MA, 02110, US

(73) Assignee: MedGene Ltd., Abingdon (GB)

(21) Appl. No.: 12/516,153

(22) PCT Filed: Nov. 8, 2007
(86) PCT No.: PCT/GB07/04274

§ 371 Date: Sep. 30, 2009

(30) Foreign Application Priority Data
Nov. 24, 2006 (GB) 0623539.4

(51) Int. Cl. A61K 39/395; C07K 16/00; A61P 37/06; A61P 11/06; A61P 17/00; A61P 31/12

(52) U.S. Cl. 424/133.1; 530/387.3; 530/391.3; 530/391.7

(57) ABSTRACT

The present invention provides monomeric and dimeric polypeptide fusions comprising mutated human ILT molecules and immunoglobulin Fc segments. Such compostions are useful, either alone or associated with a therapeutic agent, for targeting cells expressing Class I pMHC molecules.

The present invention relates to monomeric polypeptides comprising an ILT-like segment and an Fc-like C-terminal segment wherein either

(a) the ILT-like segment is the N-terminal segment of the polypeptide and has at least a 45% identity and/or 55% similarity to SEQ ID NO: 19; and said N-terminal segment per se has the property of (i) binding to a given Class I pMHC with a K_{D} of less than or equal to 1 μM and/or with an off-rate (k_{off}) of 2 S⁻¹ or slower, and (ii) inhibiting CD8 binding to the given pMHC to a greater extent than the polypeptide SEQ ID NO: 3; and the Fc-like segment is the C-terminal segment of the polypeptide and comprises a portion of the constant domain of one of the heavy chains of an immunoglobulin having at least 70% identity and/or 80% similarity to the corresponding portion of SEQ ID 139; or
(b) the Fc-like segment is the N-terminal segment of the polypeptide and comprises a portion of the constant domain of one of the heavy chains of an immunoglobulin having at least 70% identity and/or 80% similarity to the corresponding portion of SEQ ID 139; and the ILT-like segment is the C-terminal segment of the polypeptide and has at least a 45% identity and/or 55% similarity to SEQ ID NO: 19; and said C-terminal segment per se has the property of (i) binding to a given Class I pMHC with a K_{D} of less than or equal to 1 μM and/or with an off-rate (k_{off}) of 2 S⁻¹ or slower, and (ii) inhibiting CD8 binding to the given pMHC to a greater extent than the polypeptide SEQ ID NO: 3.

Also provided are said polypeptides associated with therapeutic agents, multivalent complexes of said polypeptides, and methods for using these polypeptides.

### BACKGROUND TO THE INVENTION

### ILTs

Immunoglobulin-like transcripts (ILTs) are also known as Leukocyte Immunoglobulin-like receptors (LIRs), monocyte/macrophage immunoglobulin-like receptors (MIRs) and CD85. This family of immunoreceptors form part of the immunoglobulin superfamily. The identification of ILT molecules was first published in March 1997 in a study (Samaridis et al., (1997) Eur J Immunol 27 660-665) which detailed the sequence of LIR-1 (ILT-2), noted their similarity to bovine FCγ2R, human killer cell inhibitory receptors (KIRs), human FcαR, and mouse gp49. This study also noted that LIR-1, unlike KIRs, is predominately expressed on monocytic and B lymphoid cells.

The ILT family of immunoreceptors are expressed on the surface of lymphoid and myeloid cells. The ILT molecules share 63-84% homology in their extracellular regions and all except the soluble LIR-4 are type I transmembrane proteins. All the currently identified ILT molecules have either two or four immunoglobulin superfamily domains in their extracellular regions. (Willcox et al., (2003) 4 (9) 913-919) Individual ILT molecules may also be expressed as a number of distinct variants/isoforms. (Colonna et al., (1997) J Exp Med 186 (11) 1809-1818) and (Cosman et al., (1997) Immunity 7 273-282)

There are a number of scientific papers detailing the structure and function of ILT molecules including the following: (Samaridis et al., (1997) Eur J Immunol 27 660-665), (Cella, et al., (1997) J Exp Med 185 (10) 1743-1751), (Cosman et al., (1997) Immunity 7 273-282), (Borges et al., (1997) J Immunol 159 5192-5196), (Colonna et al., (1997) J Exp Med 186 (11) 1809-1818), (Colonna et al., (1998) J. Immunol 160 3096-3100), (Cosman et al., (1999) Immunological Revs 168 177-185), (Chapman et al., (1999) Immunity 11 603-613), (Chapman et al., (2000) Immunity 12 727-736), (Willcox et al., (2002) BMC Structural Biology 2 6), (Shiroshi et al., (2003) PNAS 100 (5) 8856-8861) and (Willcox et al., (2003) 4 (9) 913-919).

WO9848017 discloses the genetic sequences encoding ILT family members and their deduced amino acid sequences. This application classified LIR molecules into three groups. The first group containing polypeptides with a transmembrane region including a positively charged residue and a short cytoplasmic tail. The second group comprising polypeptides having a non-polar transmembrane region and a long cytoplasmic tail. And finally a third group containing a polypeptide expressed as a soluble polypeptide having no transmembrane region or cytoplasmic tail. Also disclosed were processes for producing polypeptides of the LIR family, and antagonistic antibodies to LIR family members. This application discussed the possible use of LIR family members to treat autoimmune diseases and disease states associated with suppressed immune function. In this regard, it was noted that the use of soluble forms of an LIR family member is advantageous for certain applications. These advantages included the ease of purifying soluble forms of ILTs/LIRs from recombinant host cells, that they are suitable for intravenous administration and their potential use to block the interaction of cell surface LIR family members with their ligands in order to mediate a desirable immune function. The possible utility of soluble LIR fragments that retain a desired biological activity, such as binding to ligands including MHC class I molecules was also noted.

Another study (Shiroishi et al (2003) PNAS 100 (15) 8856-8861) discussed soluble (truncated) forms of ILT-2 and ILT-4 molecules. Their ability to compete with soluble CD8 for binding to MHC molecules in Biacore studies was noted and it was postulated that this may be one of the mechanisms by which ILT-2 modulates CD8+ T cell activation. In relation to pMHC binding this study states “The higher affinity of ILT versus CD8 binding suggests that ILTs may effectively block CD8 binding at the cell surface. This study noted that ILT2 binds to the α3 domain of Class I MHC and that the crystal structure of an ILT2 fragment containing domains 1 and 2 had been reported.

(Colonna et al., (1998) J. Immunol. 160 3096-3100) which focussed on ILT-4, contains a summary of the tissue distribution and specificity of ILTs 2-5. Of these ILT molecules, ILT-2 and ILT-4 are noted to bind Class I MHC molecules. This study analysed the binding of soluble ILT-4 to cells transfected with various Class I MHCs. The study concluded that ILT-4 binds to HLAs-A, B and G, but not HLA-Cw3 or HLA-Cw5.

WO03041650 discloses a method of treating Rheumatoid Arthritis (RA) using modulators of LIR-2 and/or LIR-3/LIR-7 activity. The modulators disclosed include both agonists and antagonists of LIR activity. WO2006033811 discloses the use of ILT-3 polypeptides and fusions thereof as therapeutic agents for the inhibition of graft rejection.

The affinity for various soluble analogues of Wild-Type ILT molecules for different pMHC targets has been determined. For example, (Chapman et al., (1999) Immunity 11 603-613) used Biacore-based methods to determine that LIR-1 (ILT-2) bound to a range of HLA-A, HLA-B, HLA-C, HLA-E and HLA-G molecules. The determined K_{D} values for these interactions ranged from 1×10⁻⁴ M (for HLA-G1) to 2×10⁻⁵ M (for HLA-Cw*0702). This study also noted that the K_{D} of the interaction between ILT-2 had an affinity for UL18, a viral analogue of Class I MHC, in the nM range.

A further study (Chapman et al., (2000) Immunity 12 727-736) reported the crystal structure of a truncated LIR-1 (ILT-2) polypeptide comprising the D1 and D2 domains. LIR-1 was known to bind to the UL18 viral class I MHC analogue with much higher affinity than the similar LIR-2. The authors used the crystal structure of the truncated LIR-1 polypeptide to identify differences between LIR-1 and LIR-2 that occurred in solvent-exposed residues. Site-directed mutagenesis of these two peptides was the used to confirm which residues were involved in UL18 binding. This was carried out by substituting WT residues from LIR-1 in to the corresponding amino acid positions of LIR-2. The study concluded that residue 38Y, and at least one of 76Y, 80D or 83R of LIR-1 were involved in UL18 binding. The authors stated that “Because the affinity of LIR-1 for class I MHC proteins is much lower than for UL18 we were unable to derive accurate affinities for the binding of the LIR-1 and LIR-2 mutants to class I MHC.”

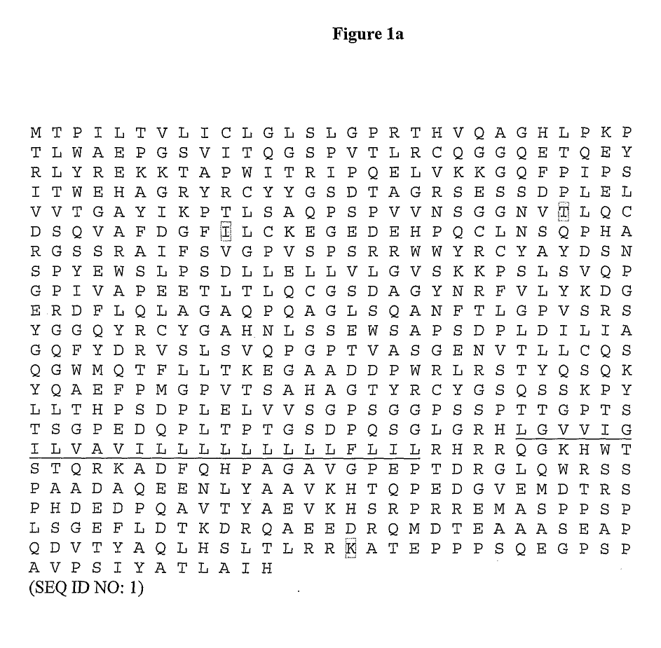

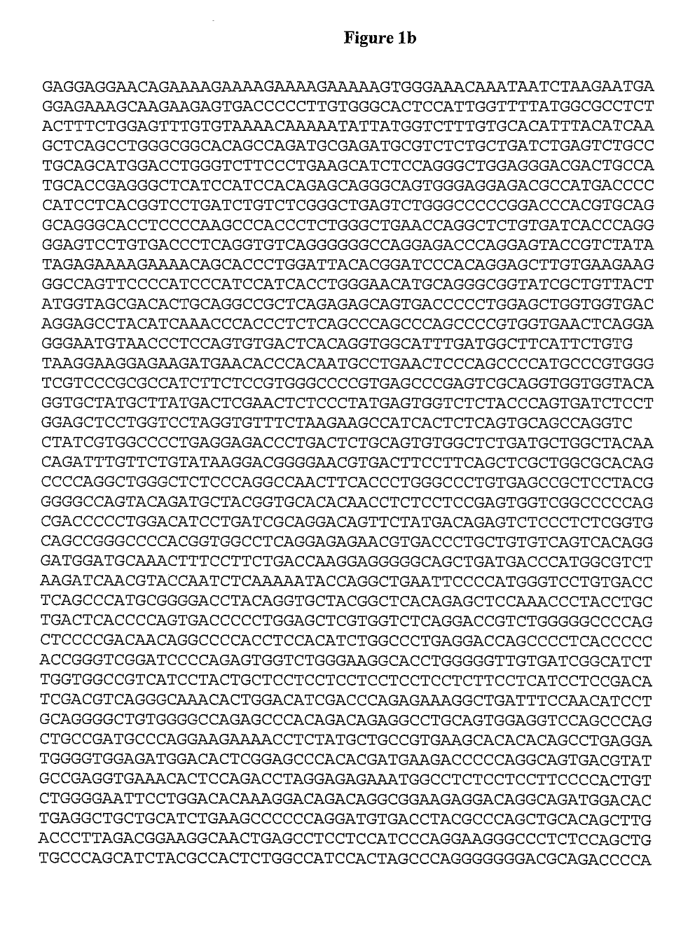

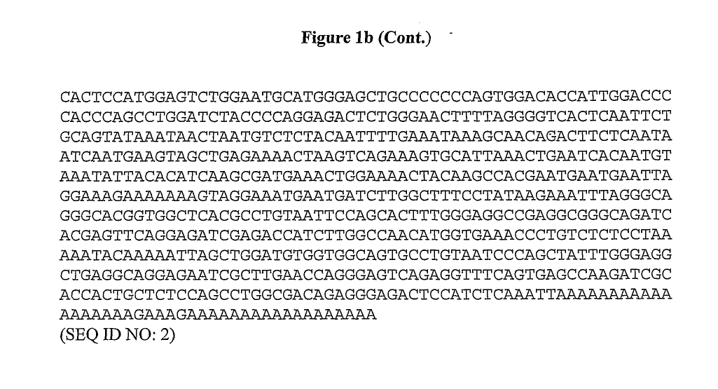

The full amino acid and DNA sequences of a Wild-Type human ILT-2 are shown in FIGS. 1a (SEQ ID NO:1) and 1b (SEQ ID NO:2) respectively. The DNA sequence provided corresponds to that given accession number NM_{—}006669 on the NCBI nucleotide database.

Our co-pending International Patent WO 2006/125963 describes and claims ILT-like polypeptides having higher affinities for Class I peptide-MHC complexes.

### Fc Fusions

As will be known to those skilled in the art the fusion of biologically active polypeptides to the Fragment Crystallisation (Fc) portion of an immunoglobulin may impart therapeutically beneficial changes to the pharmaco-kinetic (PK) properties of these biologically active polypeptides. A number of Fc fusion-based therapeutics are on the market including Abatacept®, a CTLA4-Fc fusion polypeptide.

WO 98/48017 describes the production of soluble two domain (D1D2) analogues of wild-type ILT, and Fc fusion polypeptides comprising these soluble analogues of ILTs.

Soluble polypeptide monomers and dimers such as Fc fusions with the pMHC binding characteristics of ILT molecules and multivalent complexes thereof provide a means of blocking the CD8 binding site on pMHC molecules, for example for the purpose of inhibiting CD8⁺ T cell-mediated autoimmune disease. However, for that purpose it would be desirable if these polypeptide monomers and dimers had a higher affinity and/or a slower off-rate for the target pMHC molecules than native ILT molecules.

### BRIEF DESCRIPTION OF THE INVENTION

The present invention relates to higher affinity polypeptide monomers and dimers of the kind with which our above WO 2006/125963 is concerned, but presented as Fc-fusions. Such fusions have the advantage over the non-fused ILT-like monomers and dimers, for example in terms of improved pharmacokinetic properties, such as increased plasma half-lives. The monomers and dimers of the invention may be associated with therapeutic agents, may be assembled into multivalent complexes, and may be used in the treatment of autoimmune diseases.

### DETAILED DESCRIPTION OF THE INVENTION

As noted above ILT molecules are also known as LIRs, MIRs and CD85. The term ILT as used herein is understood to encompass any polypeptide within this family of immunoreceptors.

### Polypeptide Monomers and Dimers Comprising High Affinity ILT-Like Polypeptides and Fc-Like Portions

The present invention provides monomeric polypeptides comprising an ILT-like segment and an Fc-like C-terminal segment wherein either

(a) the ILT-like segment is the N-terminal segment of the polypeptide and has at least a 45% identity and/or 55% similarity to SEQ ID NO: 19; and said N-terminal segment per se has the property of (i) binding to a given Class I pMHC with a K_{D} of less than or equal to 1 μM and/or with an off-rate (k_{off}) of 2 S⁻¹ or slower, and (ii) inhibiting CD8 binding to the given pMHC to a greater extent than the polypeptide SEQ ID NO: 3; and the Fc-like segment is the C-terminal segment of the polypeptide and comprises a portion of the constant domain of one of the heavy chains of an immunoglobulin having at least 70% identity and/or 80% similarity to the corresponding portion of SEQ ID 139; or
(b) the Fc-like segment is the N-terminal segment of the polypeptide and comprises a portion of the constant domain of one of the heavy chains of an immunoglobulin having at least 70% identity and/or 80% similarity to the corresponding portion of SEQ ID 139; and the ILT-like segment is the C-terminal segment of the polypeptide and has at least a 45% identity and/or 55% similarity to SEQ ID NO: 19; and said C-terminal segment per se has the property of (i) binding to a given Class I pMHC with a K_{D} of less than or equal to 1 μM and/or with an off-rate (k_{off}) of 2 S⁻¹ or slower, and (ii) inhibiting CD8 binding to the given pMHC to a greater extent than the polypeptide SEQ ID NO: 3.

The present invention also provides polypeptide dimers comprising a first polypeptide and a second polypeptide, in which dimer

(i) the first and/or the second polypeptide comprises an ILT-like segment having at least a 45% identity and/or 55% similarity to SEQ ID NO: 19;
(ii) said ILT-like segment(s) per se having the property of (a) binding to a given Class I pMHC with a K_{D} of less than or equal to 1 μM and/or with an off-rate (k_{off}) of 2 S⁻¹ or slower, and (b) inhibiting CD8 binding to the given pMHC to a greater extent than the polypeptide SEQ ID NO: 3;
(iii) each of the first and second polypeptides comprises an Fc-like segment comprising a portion of the constant domain of one of the heavy chains of an immunoglobulin having at least 70% identity and/or 80% similarity to the corresponding portion of SEQ ID NO: 139;
and wherein either (a) the ILT-like segment(s) is/are the N-terminal segment(s) of the first and/or second polypeptides, and the Fc-like segments are the C-terminal segments of the first and second polypeptides or (b) the Fc-like segments are the N-terminal segments of the first and/or second polypeptides, and the ILT-like segment(s) is/are the C-terminal segment(s) of the first and second polypeptides.

One embodiment is provided by polypeptide dimers of the invention wherein the ILT-like segment(s) is/are the N-terminal segment(s) of the first and/or second polypeptides, and the Fc-like segments are the C-terminal segments of the first and second polypeptides

Polypeptide monomers and dimers which meet the above homology and Class I pMHC-binding criteria may be regarded as polypeptide monomers comprising high affinity ILT-like portions and Fc-like portions and may be referred to herein as such.

### FC-Like Segments of the Polypeptide Monomers and Dimers of the Invention

In one broad aspect the polypeptide dimers of the invention comprise at least one inter-chain covalent link between a residue in one of the said Fc-like segments and a residue in the other said Fc-like residue. These inter-chain covalent links may correspond to links present between cysteine residues in the heavy chain constant domains of native immunoglobulins and/or non-native interchain links may be introduced.

A further aspect is provided by polypeptide monomers or dimers of the invention having the property of binding to an Fc receptor via the said Fc-like segments. The ability of the polypeptide dimers of the invention to bind to a given Fc receptor can be may be assessed by any suitable means. Example 8 herein provides a Fluorescence Activated Cell Sorting (FACS) based competitive binding assay for assessing this ability.

Polypeptide monomers or dimers of the invention wherein the Fc-like segment or segments comprise respectively one or both of the chains of the Fc portion of an immunoglobulin provide another aspect of the invention. Such Fc portions can be comprised of the CH2 and CH3 domains of an immunoglobulin and optionally the hinge region of the immunoglobulin. The Fc fragment can be of an IgG, an IgA, an IgM, an IgD, or an IgE.

Preferred embodiments of the present aspect are provided wherein the said immunoglobulin is an IgG immunoglobulin. For example the said immunoglobulin may an IgG1 immunoglobulin, such as human IgG1 immunoglobulin.

In a further preferred embodiment of the present aspect the Fc-like segment or segments comprise respectively one or two of amino acid sequence SEQ ID NO: 139.

Another broad aspect is provided polypeptide monomers or dimers of the invention, wherein the Fc-like segment or segments comprise respectively one or both of the chains of a mutated Fc portion of an immunoglobulin.

As will be obvious to those skilled in the art the mutation(s) in these Fc portion amino acid sequences may be one or more of substitution(s), deletion(s) or insertion(s). These mutations can be carried out using any appropriate method including, but not limited to, those based on polymerase chain reaction (PCR), restriction enzyme-based cloning, or ligation independent cloning (LIC) procedures. These methods are detailed in many of the standard molecular biology texts. For further details regarding polymerase chain reaction (PCR) mutagenesis and restriction enzyme-based cloning see (Sambrook & Russell, (2001) Molecular Cloning—A Laboratory Manual (3^{rd} Ed.) CSHL Press) Further information on LIC procedures can be found in (Rashtchian, (1995) Curr Opin Biotechnol 6 (1): 30-6)

Such mutations may be introduced for a number of reasons. For example it may de desirable to introduce mutations to the said Fc-like segment(s) which impact one or more of disulfide bond formation, expression levels achievable in a selected host cell, N-terminal heterogeneity upon expression in a selected host cell, Fc portion glycosylation or the level of antibody-dependent cellular cytotoxicity (ADCC) and/or complement-dependent cellular cytotoxicity (CDCC) responses to the polypeptide monomers and dimers of the invention. WO2005073383 provides a detailed discussion of mutations of the above types.

Specific embodiments of the present aspect are provided by polypeptide monomers or dimer of the invention mutated so as to reduce antibody-dependent cellular cytotoxicity (ADCC) and/or complement-dependent cellular cytotoxicity (CDCC) responses thereto wherein the said Fc-like segment or segments has or have a sequence or sequences corresponding to SEQ ID NO: 139 in which one or more of amino acids corresponding to amino acids 13E, 14L, 15L, 16G, 107A, 110A or 111P of SEQ ID NO: 139 is/are mutated. For example, wherein the said Fc-like segment or segments has or have a sequence or sequences corresponding to SEQ ID NO: 139 having one or more of the following mutations 13E→P, 14L→V, 15L-A, deletion of 16G, 107A→G, 110A→S or 111P→S using the numbering of SEQ ID NO: 139.

Further embodiments of the present aspect are provided by polypeptide monomers or dimers of the invention wherein the said Fc-like segment or segments is/are mutated so as so as to increase the plasma half-life of the monomer or dimer. Specific embodiments of the present aspect are provided by polypeptide monomers or dimer of the invention wherein the said Fc-like segment or segments has or have a sequence or sequences corresponding to SEQ ID NO: 139 in which one or both of amino acids corresponding to amino acids 30T and 208M is/are mutated. For example, wherein the said Fc-like segment or segments has or have a sequence or sequences corresponding to SEQ ID NO: 139 having one or more of the following mutations 30T→Q or 208M→L using the numbering of SEQ ID NO: 139. IgG1 antibodies containing these Fc mutations have been shown to have serum half-lives in rhesus monkeys than the corresponding wild-type antibodies. The increased serum half-lives of antibodies incorporating these mutations is believed to be due to their increased affinity for the human neo-natal FC receptor (FcRn) which, in turn, is believed to allow these mutated antibodies to avoid lysosomal degradation and to be returned into the circulation. (Hinton et al., (2005) J. Immunol. 176: 346-356)

The term “correspondence” as used herein between two sequences need not be 1:1 on an amino acid level. N- or C-truncation, and/or amino acid deletion and/or substitution relative to the corresponding human ILT2 sequence is acceptable, provided the overall result is preserved orientation of sequence as in native ILT and retention of peptide-MHC binding functionality. In particular, the sequences present in the mutated ILT molecules that are not directly involved in contacts with the peptide-MHC complex to which the mutated ILT molecules bind, they may be shorter than, or may contain substitutions or deletions relative to the sequence of native ILT2.

Preferably the Fc-like segment(s) of the polypeptide monomers and dimers of the invention are CHARACTERISED IN THAT said segment(s) have at least a 80% identity and/or 90% similarity to SEQ ID NO: 139.

Preferably the Fc-like segment(s) of the polypeptide monomers and dimers of the invention are CHARACTERISED IN THAT said segment(s) have at least a 90% identity and/or 95% similarity to SEQ ID NO: 139.

Preferably the Fc-like segment(s) of the polypeptide monomers and dimers of the invention are CHARACTERISED IN THAT said segment(s) have at least a 95% identity and/or 98% similarity to SEQ ID NO: 139.

Sequence identity as used herein means identical amino acids at corresponding positions in the two sequences which are being compared. Similarity in this context includes amino acids which are identical and those which are similar (functionally equivalent). For example a single substitution of one hydrophobic amino acid present at a given position in a polypeptide with a different hydrophobic amino acid would result in the formation of a polypeptide which was considered similar to the original polypeptide but not identical). The parameters “similarity” and “identity” as used herein to characterise polypeptides of the invention are determined by use of the FASTA algorithm as implemented in the FASTA programme suite available from William R. Pearson, Department of Biological Chemistry, Box 440, Jordan Hall, Charlottesville, Va. The settings used for determination of those parameters via the FASTA programme suite are as specified in Example 6 herein.

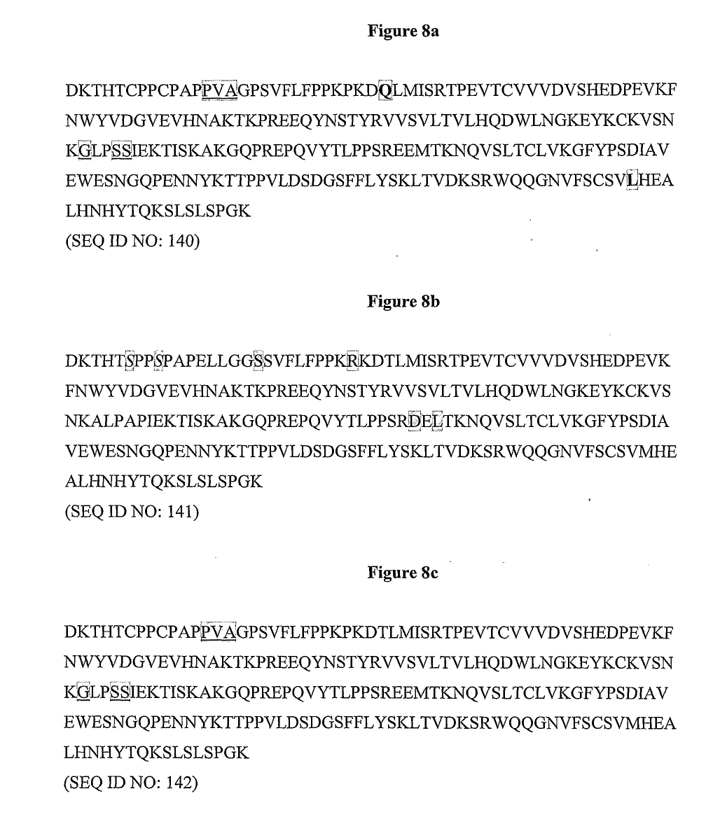

Further specific embodiments are provided by polypeptide monomers or dimers of the invention, wherein the Fc-like segment, or both Fc-like segments, comprise the amino acid sequence of any of SEQ ID NOs: 140-143. (See FIGS. 8a-8d respectively for the amino acid sequences of these polypeptides)

### ILT-Like Segments of the Monomers and Dimers of the Invention

The ILT-like segment(s) of the polypeptide monomers or dimers of the invention are either high affinity ILT-like polypeptides, or are functional equivalents thereof.

As stated above, naturally occurring ILT polypeptides have either two or four immunoglobulin superfamily domains in their extracellular regions. The ILT-like segments of the polypeptide monomers and dimers of the invention comprise high affinity ILT-like polypeptides which may be expressed in forms having four, three or two of said domains. The currently preferred embodiments of the invention are polypeptide dimers comprising two immunoglobulin superfamily domains corresponding to the two N-terminal domains of human ILT-2 containing one or more mutation(s) which confer high affinity for Class I pMHC. These N-terminal domains are domains one and two using the notation of Cosman et al., (1999) Immunol Revs 168: 177-185. ILT-like segments having those two N-terminal domains generally have a sequence corresponding to amino acids 1-195 of SEQ ID NO: 3.

One embodiment of the invention is provided wherein the said ILT-like segment(s) in the polypeptide monomers and dimers of the invention is/are mutated human ILT molecule(s). For example, the DNA encoding human ILT-2, or soluble fragments thereof, can be used as a template into which the various mutations that cause high affinity and/or a slow off-rate for the interaction between the high affinity ILT-like N-terminal segments(s) of the invention and the target pMHC complex can be introduced. Thus the invention includes high affinity ILT-like segments(s) which comprise ILT-2 variants which are mutated relative to the native sequence.

As will be obvious to those skilled in the art the mutation(s) in such human ILT-2 amino acid sequences may be one or more of substitution(s), deletion(s) or insertion(s). These mutations can be carried out using any appropriate method including, but not limited to, those based on polymerase chain reaction (PCR), restriction enzyme-based cloning, or ligation independent cloning (LIC) procedures. These methods are detailed in many of the standard molecular biology texts. For further details regarding polymerase chain reaction (PCR) mutagenesis and restriction enzyme-based cloning see (Sambrook & Russell, (2001) Molecular Cloning—A Laboratory Manual (3^{rd} Ed.) CSHL Press) Further information on LIC procedures can be found in (Rashtchian, (1995) Curr Opin Biotechnol 6 (1): 30-6)

For example, polypeptides comprising at least two, three, four, five, six, seven, eight, or nine of the above mutations will often be suitable.

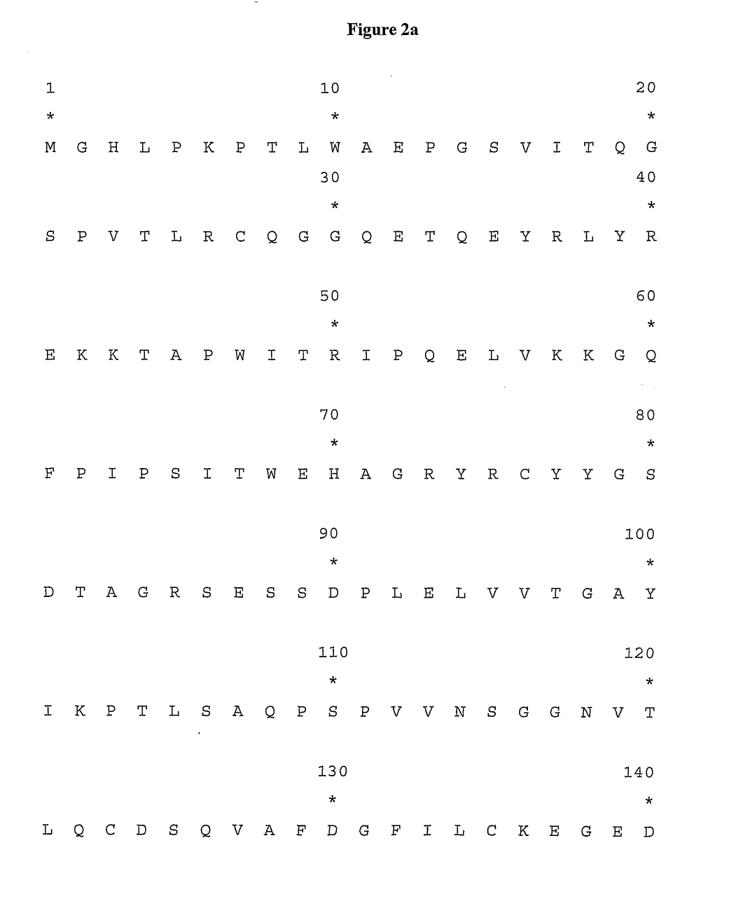

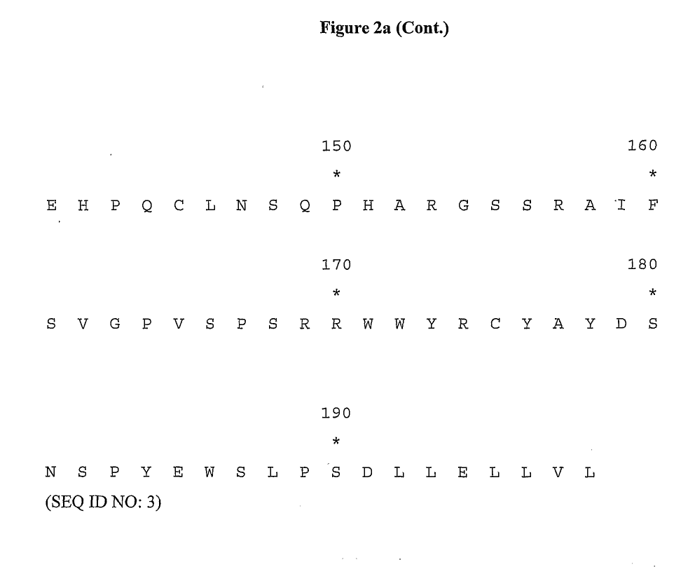

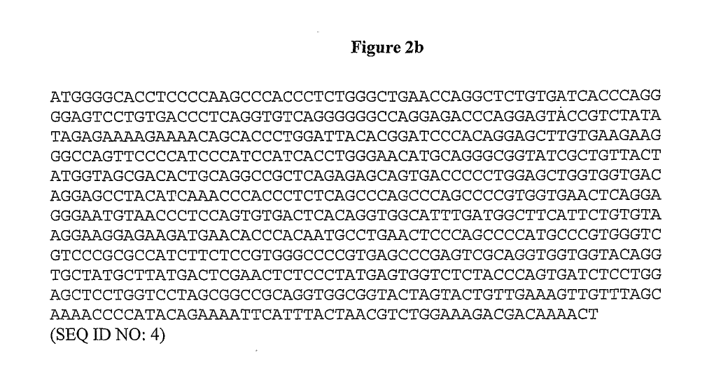

The numbering used is the same as that shown in FIG. 2a (SEQ ID No: 3).

The ILT-like segments disclosed herein are generally provided with an N-terminal Methionine (Met or M) residue which is used for expression in bacteria. As will be known to those skilled in the art this residue may be removed during the production of recombinant proteins, for example this methionine would not normally be present in mutated human ILT molecules expressed by eukaryotic cells. Another embodiment is provided by polypeptide monomers and dimers of the invention comprising said ILT-like segment(s) having amino acids corresponding to at least amino acids 3-195 of SEQ ID No: 3. Such ILT-like segments are two-domain embodiments comprising domains corresponding to the two N-terminal immunoglobulin superfamily domains of human ILT-2.

One broad aspect is provided by polypeptide monomers or dimers of the invention, wherein one or more amino acids of the ILT-like segment or segments corresponding to amino acids 10W, 19Q, 20G, 21S, 23V, 35E, 42K, 47W, 50R, 66I, 77Y, 78Y, 79G, 80S, 81D, 82T, 83A, 84G, 85R, 87E, 99A, 101I, 102K, 126Q, 127V, 128A, 129F, 130D, 141E, 146L, 147N, 159I, 168S 170R, 172W, 174R 179D, 180S, 181N, 182S, 187S, 188L, 189P, 196L or 198L of SEQ ID NO: 3 is/are mutated. Certain embodiments of the present aspect include polypeptide monomers or dimers of the invention, wherein the ILT-like segment, or both ILT-like segments, comprise one or more of the following mutations 10W→L, 19Q→M, 19Q→L, 19Q→V, 20G→D, 20G→M, 20G→Q, 20G→F, 20G→S, 20G→E, 20G→R, 21S→Q, 21S→R, 21S→A, 21S→S, 23V→L, 35E→Q, 42K→R, 47W→Q, 50R→L, 66L→V, 77Y→V, 77Y→M, 77Y→I, 77Y→Q, 78Y→Q, 78Y→I, 78Y→G, 79G→Q, 79G→Y, 79G→W, 79G→R, 79G→V, 80S→R, 80S→T, 80S→G, 81D→G, 81D→Q, 81D→L, 81D→V, 82T→G, 82T→E, 83A→S, 83A→G, 83A→R, 84G→L, 84G→Q, 84G→A, 85R→W, 87E→A, 99A→I, 99A→Y, 101I→L, 101I→K, 101I→Q, 101→V, 102K→Q, 102K→A, 102K→R, 126Q→P, 126Q→M, 127V→W, 127V→F, 128A→D, 128A→S, 128A→T, 128A→Y, 128A→V, 128A→L, 128A→Q, 128A→I, 129F→A, 129F→T, 129F→S, 129F→V, 130D→E, 141E→G, 141E→D, 146L→D, 147N→S, 159I→E, 168S→G, 170R→K, 172W→R, 174R→W, 179D→P, 179D→V, 179D→M, 179D→T, 179D→G, 180S→I, 180S→A, 180S→N, 180S→D, 180S→W, 180S→R, 180S→E, 181N→W, 181N→F, 181N→Y, 182S→T, 182S→A, 182S→W, 182S→F, 182S→L, 187S→T 188L→D, 188L→R, 188L→S, 188L→T, 188L→Q, 189P→G, 189P→M, 189P→S, 196L→D or 198L→D using the numbering of SEQ ID NO: 3.

For example, polypeptide monomer or dimers of the invention comprising at least two, three, four, five, six, seven, eight or nine of the above mutations will often be suitable.

Certain preferred embodiments are provided by polypeptide monomers or dimers of the invention, wherein the N-terminal segment(s) of the first and/or second polypeptide(s) consist(s) of or include(s) at least amino acids 3 to 195 of any of SEQ ID Nos: 6 to 136 using the numbering of SEQ ID NO: 3.

Other preferred embodiments are provided by polypeptide monomers or dimers of the invention, wherein the ILT-like segment, or both ILT-like segments, comprise mutations corresponding to 19Q→M, 20G→D, 21→Q, 83A→S, 84G→Q, 85R→W, 87E→A, 99A→V, 179D→M, 181N→W, 182S→A, 196L→D and 198L→D using the numbering of SEQ ID NO: 3. For example, wherein the ILT-like segment, or both ILT-like segments, consist of or include SEQ ID NO: 19.

Other preferred embodiments are provided by polypeptide monomers or dimers of the invention, wherein the ILT-like segment, or both ILT-like segments, comprise mutations corresponding to 19Q→M, 20G→D, 21S→Q, 35E→Q 83A→R, 84G→Q, 85R→W, 87E→A, 99A→V, 141E→D, 196L→D and 198L→D using the numbering of SEQ ID NO: 3. For example, wherein the ILT-like segment, or both ILT-like segments, consist of or include a sequence selected from the group consisting of:

at least amino acids 3 to 195 of SEQ ID NO: 19 using the numbering of SEQ ID NO: 3;
at least amino acids 3 to 195 of SEQ ID NO: 123 using the numbering of SEQ ID NO: 3; and
at least amino acids 3 to 195 of SEQ ID NO: 131 using the numbering of SEQ ID NO: 3.

A further aspect is provided by polypeptide dimers of the invention which are homodimers.

Another aspect is provided by polypeptide monomers or dimers of the inventions comprising any one of the polypeptide monomers sequences of SEQ ID Nos: 144 to 167. For example a polypeptide monomer or dimer of the invention comprising a polypeptide monomer selected from the group consisting of:

### SEQ ID No: 150;

### SEQ ID NO: 158; and

### SEQ ID NO: 166.

Preferably the ILT-like terminal segment(s) of the polypeptide monomers or dimers of the is/are CHARACTERISED IN THAT said segment(s) have at least a 60% identity and/or 75% similarity to SEQ ID NO: 19.

Preferably the ILT-like terminal segment(s) of the polypeptide monomers or dimers of the is/are CHARACTERISED IN THAT said segment(s) has at least a 75% identity and/or 85% similarity to SEQ ID NO: 19.

Preferably the ILT-like terminal segment(s) of the polypeptide monomers or dimers of the is/are CHARACTERISED IN THAT said segment(s) has at least a 90% identity and/or 95% similarity to SEQ ID NO: 19.

Sequence identity as used herein means identical amino acids at corresponding positions in the two sequences which are being compared. Similarity in this context includes amino acids which are identical and those which are similar (functionally equivalent). For example a single substitution of one hydrophobic amino acid present at a given position in a polypeptide with a different hydrophobic amino acid would result in the formation of a polypeptide which was considered similar to the original polypeptide but not identical). The parameters “similarity” and “identity” as used herein to characterise polypeptides of the invention are determined by use of the FASTA algorithm as implemented in the FASTA programme suite available from William R. Pearson, Department of Biological Chemistry, Box 440, Jordan Hall, Charlottesville, Va. The settings used for determination of those parameters via the FASTA programme suite are as specified in Example 6 herein.

As will be obvious to those skilled in the art there are a number of sources of FASTA protein: protein comparisons which could be used for this analysis. (Pearson et al., (1988) PNAS 85 2444-2448) provides further details of the FASTA algorithm.

The relative inhibitory activities of the polypeptide of SEQ ID NO 3 and any given ILT-like segment(s) contained within putative polypeptide monomers or dimers of the invention may be determined by any conventional assay from which the read-out is related to the binding affinity of CD8 for the given pMHC. In general the read-out will be an IC₅₀ value. The CD8 binding inhibition provided by the test ILT-like segment(s) and that of SEQ ID NO: 3 will be assessed at comparable concentrations and their respective IC₅₀'s determined by reference to the inhibition curves plotted from the individual results. A suitable assay is that described in Example 5 herein.

Preferably the ILT-like segment(s) of the polypeptide monomers and dimers of the invention is/are CHARACTERISED IN THAT said segment(s) has/have a K_{D} for the said given Class I pMHC of less than or equal to 100 nM and/or has an off-rate (k_{off}) for the said given Class I pMHC of 0.1 S⁻¹.

A further embodiment is provided wherein the polypeptide monomers and dimers of the invention have the properties of (a) binding to a given Class I pMHC with a K_{D} of less than or equal to 1 μM and/or with an off-rate (k_{off}) of 2 S⁻¹ or slower, and (b) inhibiting CD8 binding to the given pMHC to a greater extent than the polypeptide SEQ ID NO: 3.

As will be known to those skilled in the art there are a number of means by which said affinity and/or off-rate can be determined. For example, said affinity (K_{D}) and/or off-rate (k_{off}) may be determined by Surface Plasmon Resonance. Example 4 herein provides a Biacore-based assay suitable for carrying out such determinations.

For comparison the interaction of a soluble truncated variant of the Wild-Type ILT-2 molecule (see FIG. 2a (SEQ ID NO: 3) for the amino acid sequence of this soluble polypeptide) and HLA-A*0201 loaded with the Carcinoembryonic antigen (CEA)-derived YLSGANLNL (SEQ ID NO: 138) peptide has a K_{D} of 6 μM, and an off-rate (k_{off}) of 2.4 S⁻¹ as measured by the Biacore-based method of Example 4. This soluble ILT-2 molecule is a truncated form of a variant of isoform 1 of Wild Type human ILT-2 which contains only extracellular domains D1 and D2. The amino acid residues which differ between this ILT-2 variant molecule and those of isoform 1 of ILT-2 are highlighted in FIG. 1a.

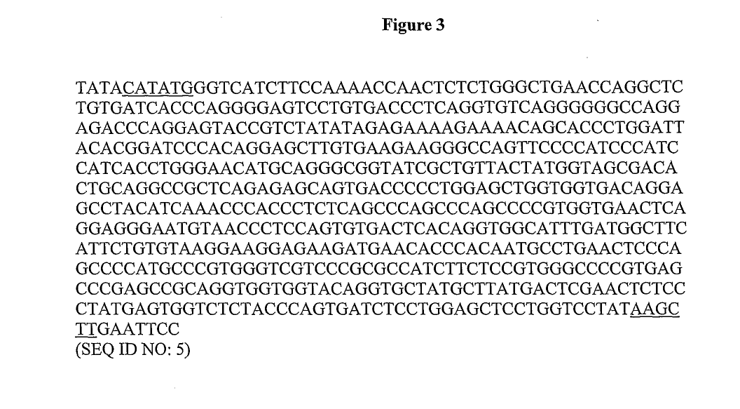

FIG. 2b (SEQ ID NO: 4) details the native DNA sequence encoding this polypeptide. In order to improve the efficiency of recombinant expression and to facilitate cloning of this polypeptide a number of mutations were introduced into the DNA encoding this polypeptide. These mutations do not alter the amino acid sequence of the expressed polypeptide. The DNA sequence used for recombinant expression is shown in FIG. 3 (SEQ ID NO: 5)

Those skilled in the art will appreciate that it is inevitable that there will be minor amino acid substitutions, deletions and insertions which do not affect the overall identity and properties of the embodiment. In particular, it should be noted that truncations of 1, 2, 3, 4 or 5 amino acids at the N-terminus of the C-terminal and/or N-terminal segments of the first and/or second polypeptides of the polypeptide dimers of the invention are unlikely to impair the functionality of said polypeptide dimers. Such minor variations may be regarded as phentoypically silent variations of such polypeptides. Looked at another way, such variations result in a segment which has the same function as the parent and achieves that function in the same way,

Polypeptide Monomers and Dimers of the Invention with Enhanced Solubility

The polypeptide monomers and dimers of the invention may be soluble, and these soluble polypeptides may be used as therapeutics. In such instances is desirable to increase the solubility of said polypeptide monomers and dimers. The invention encompasses polypeptide monomers and dimers which comprise one or more mutation(s) which increase the solubility of the polypeptide relative to a corresponding polypeptide dimer lacking said mutations. As will be known to those skilled in the art when increased solubility of a polypeptide is sought it is generally preferable to mutate amino acids which are solvent exposed. These solvent exposed amino acids in the ILT-like segment(s) of polypeptide monomers and dimers of the invention can be identified by reference to the crystal structure of ILT-2. (See Chapman et al., (2000) Immunity 12 727-736) The invention encompasses polypeptide monomers or dimers of the invention wherein one or more solvent-exposed amino acid(s) are mutated. For example, polypeptide monomers or dimers of the invention comprising at least one mutation wherein a solvent exposed hydrophobic amino acid is substituted by a charged amino acid.

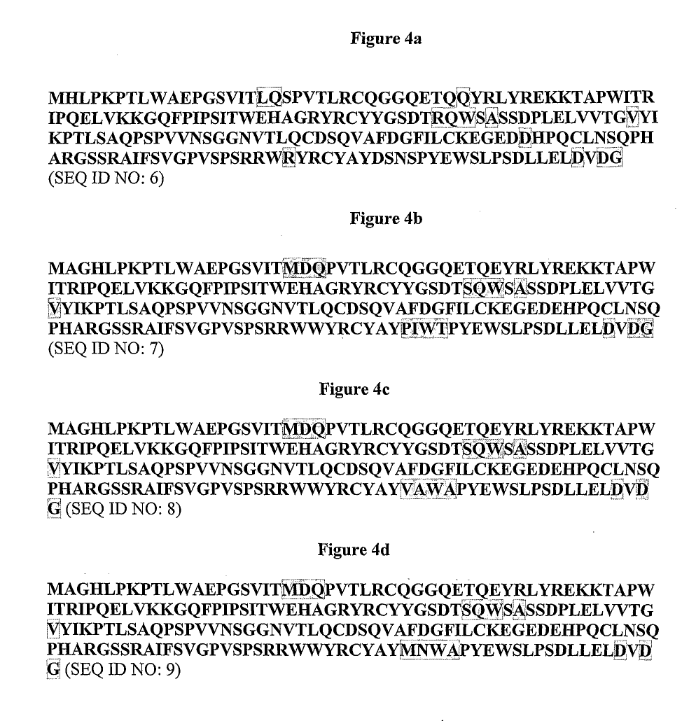

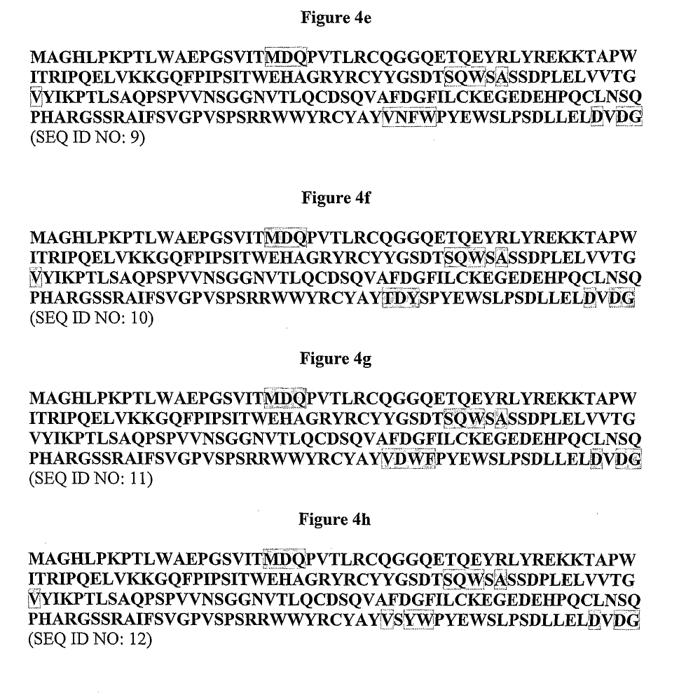

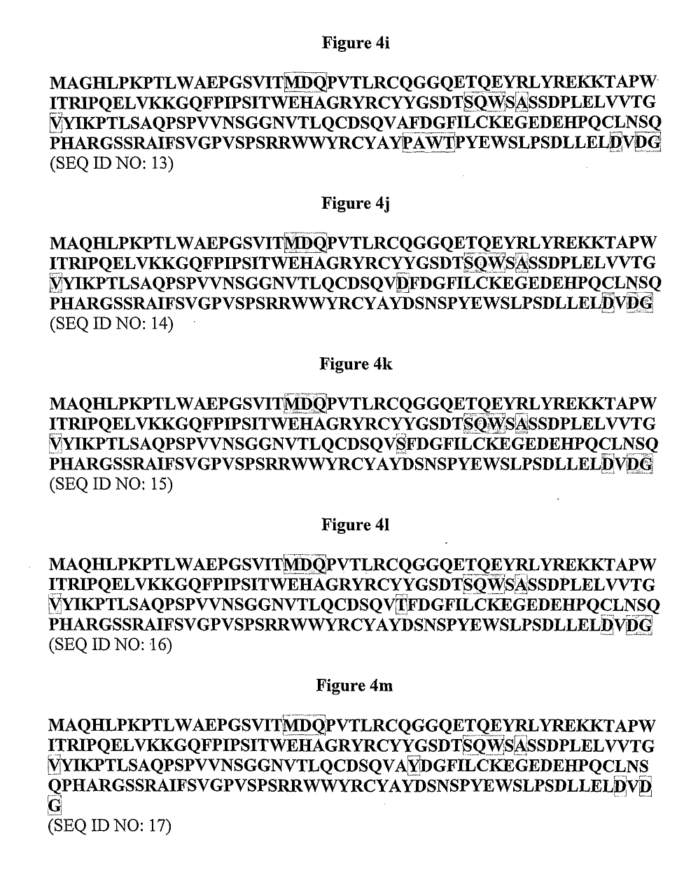

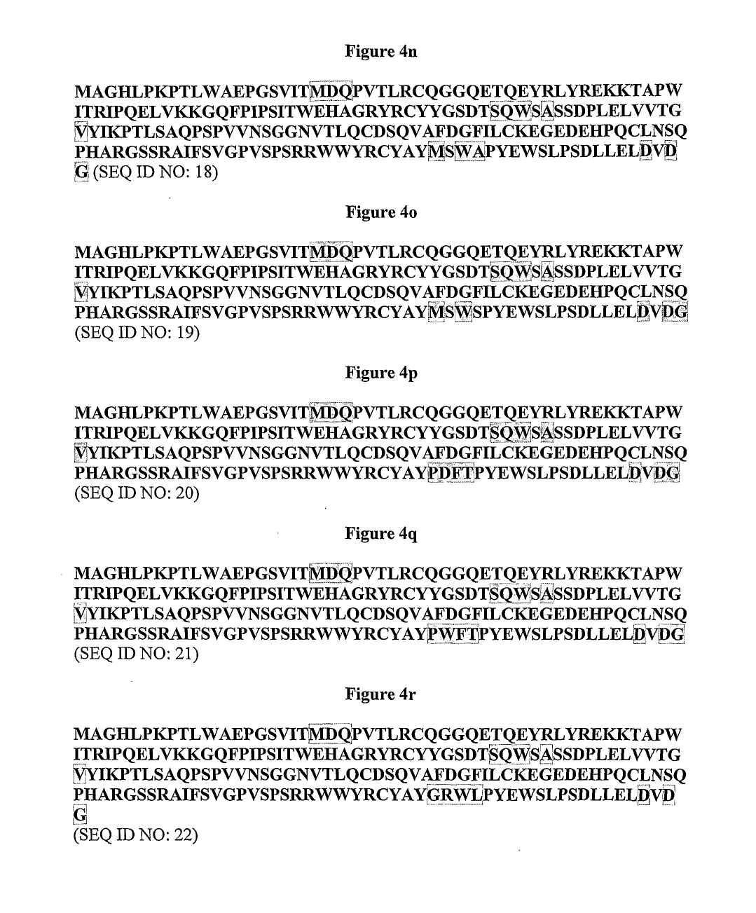

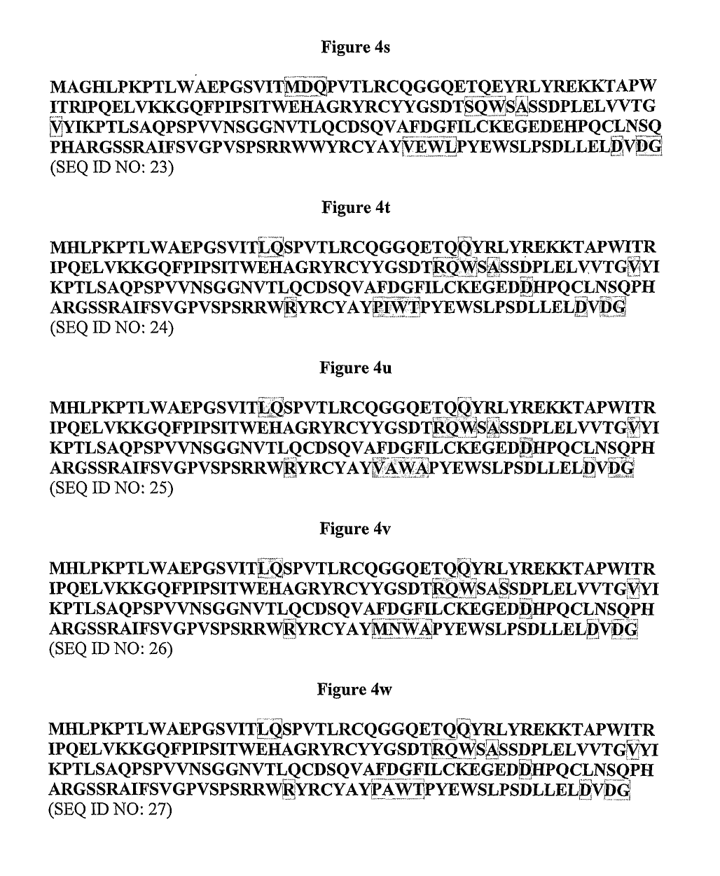

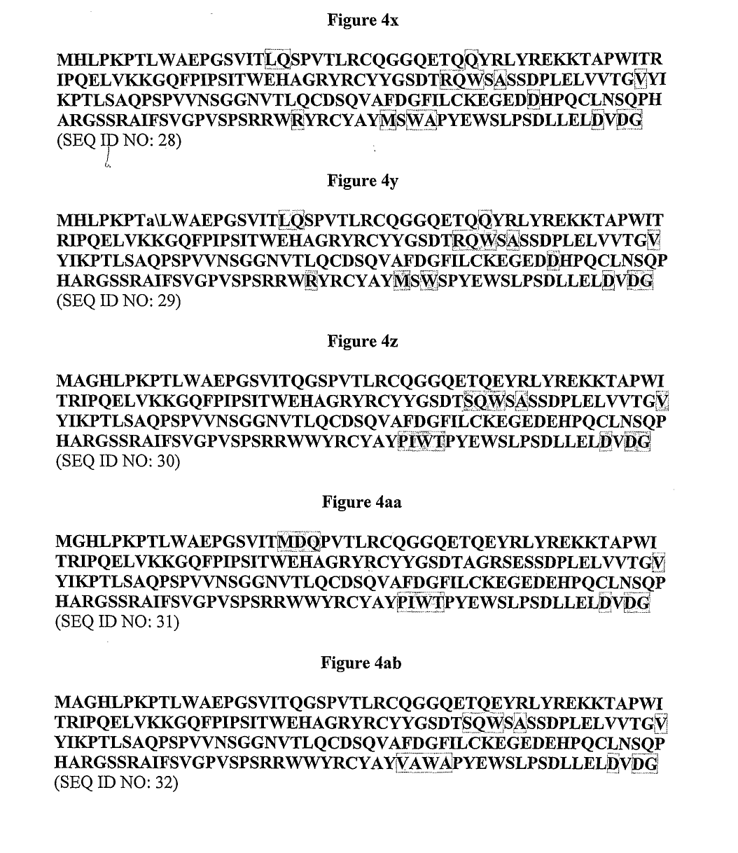

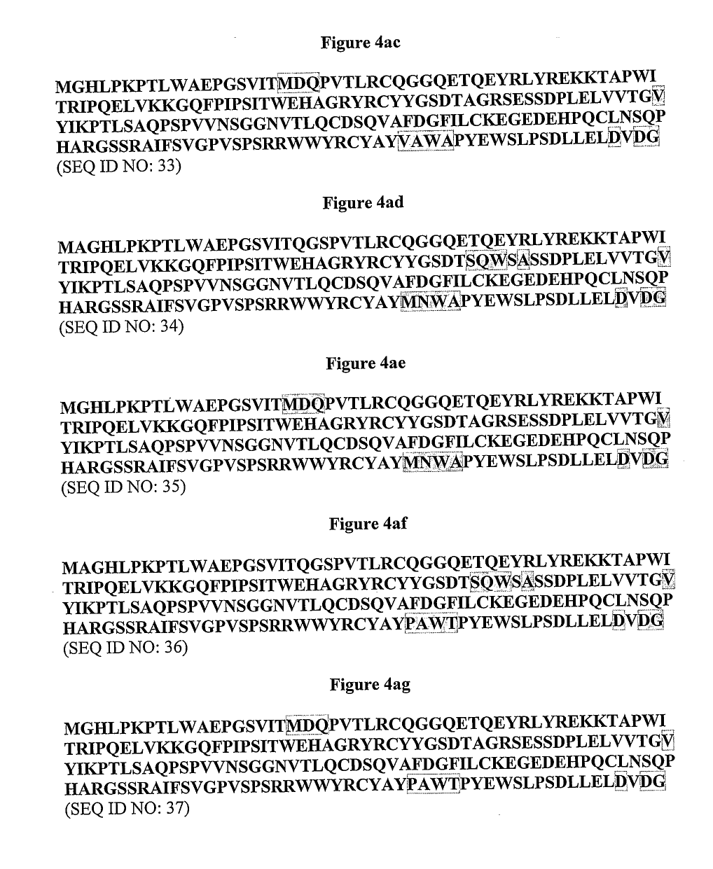

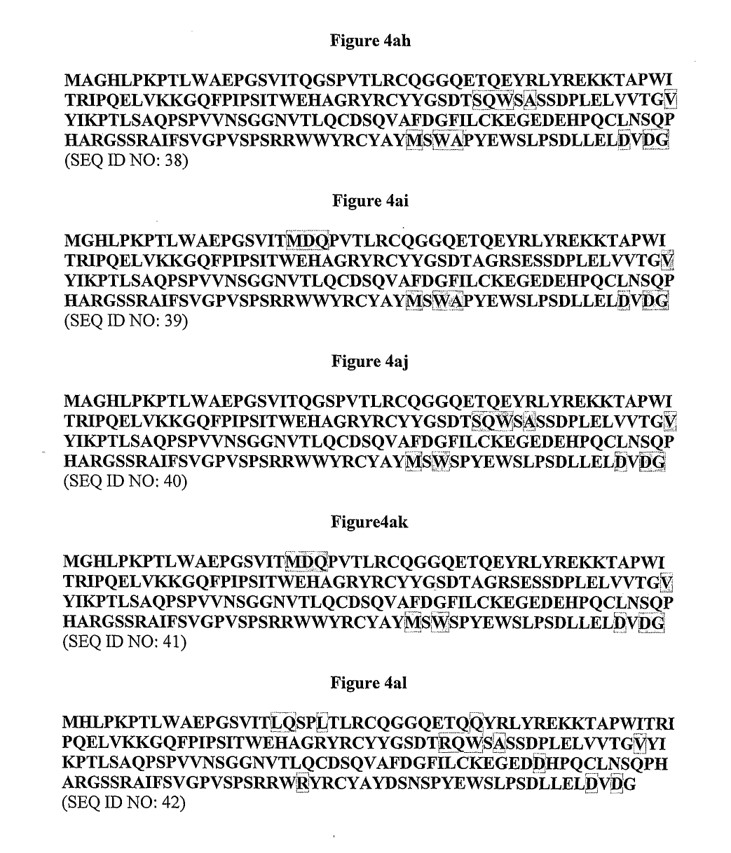

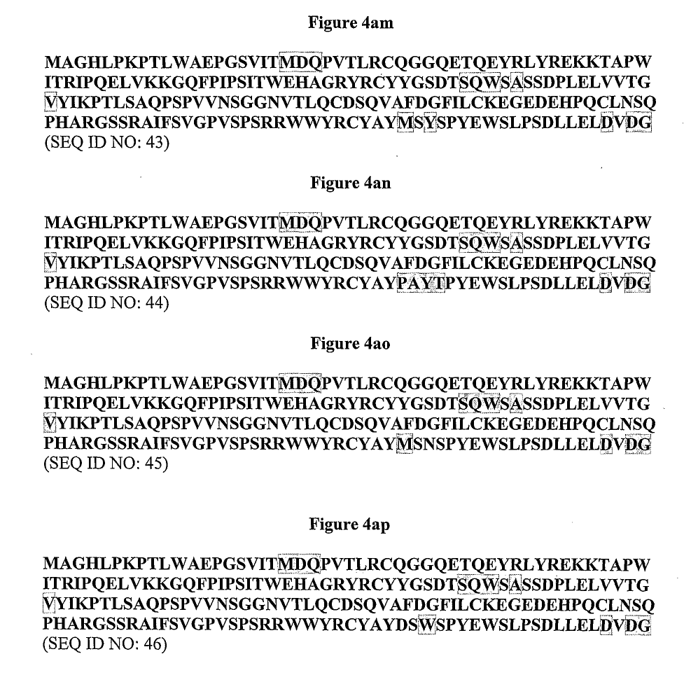

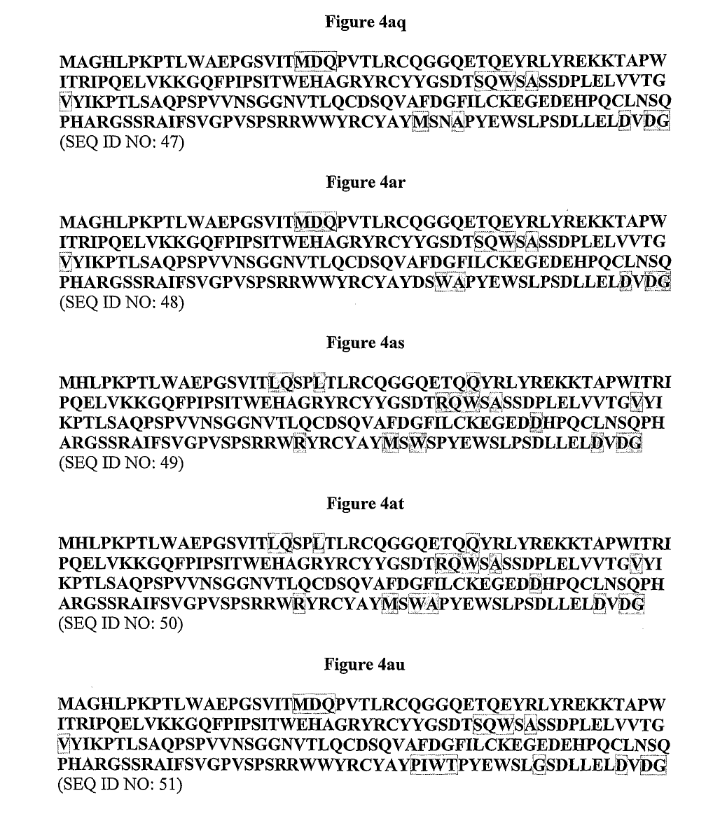

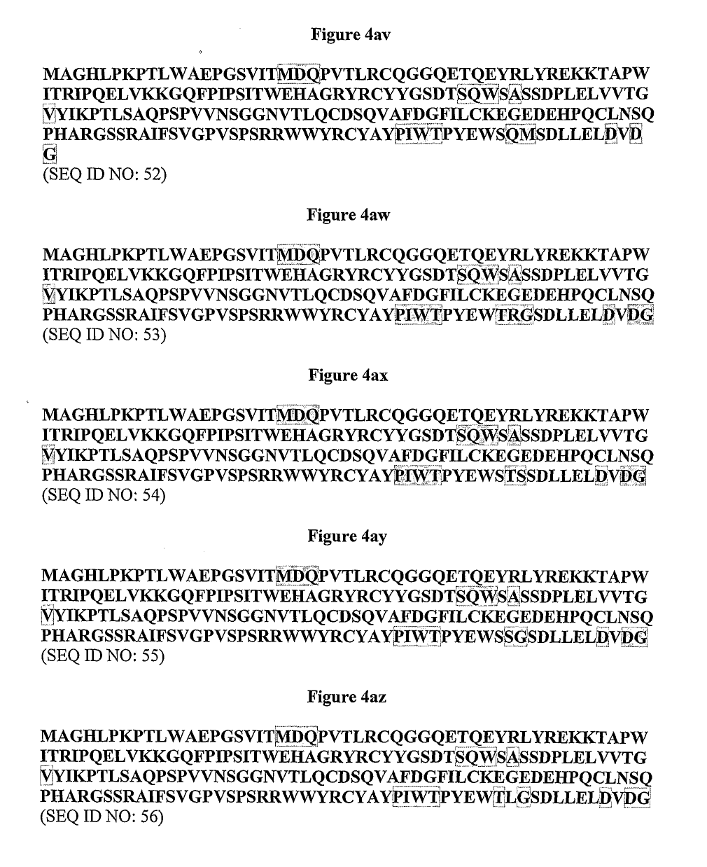

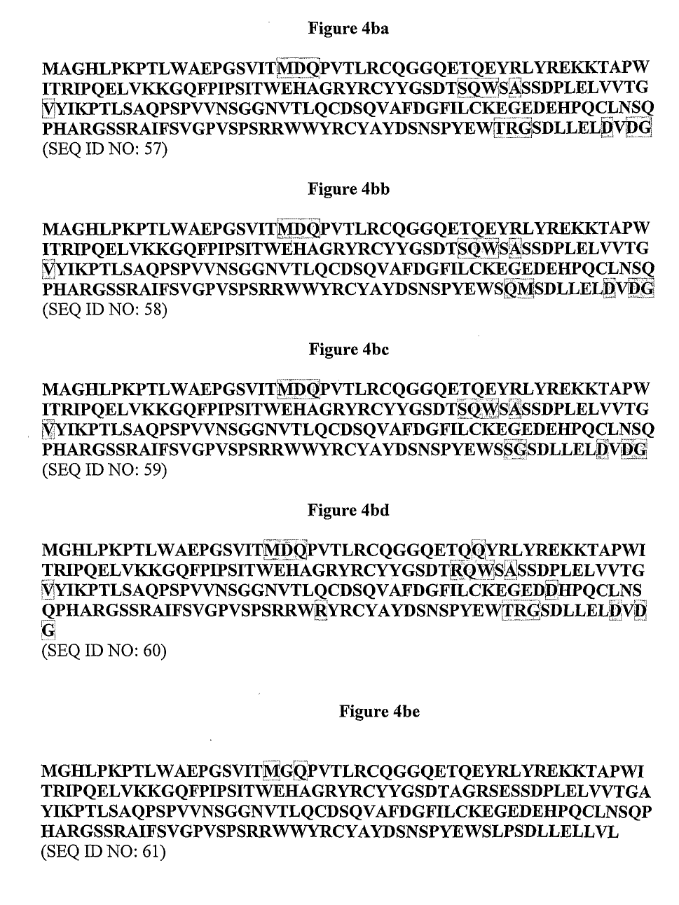

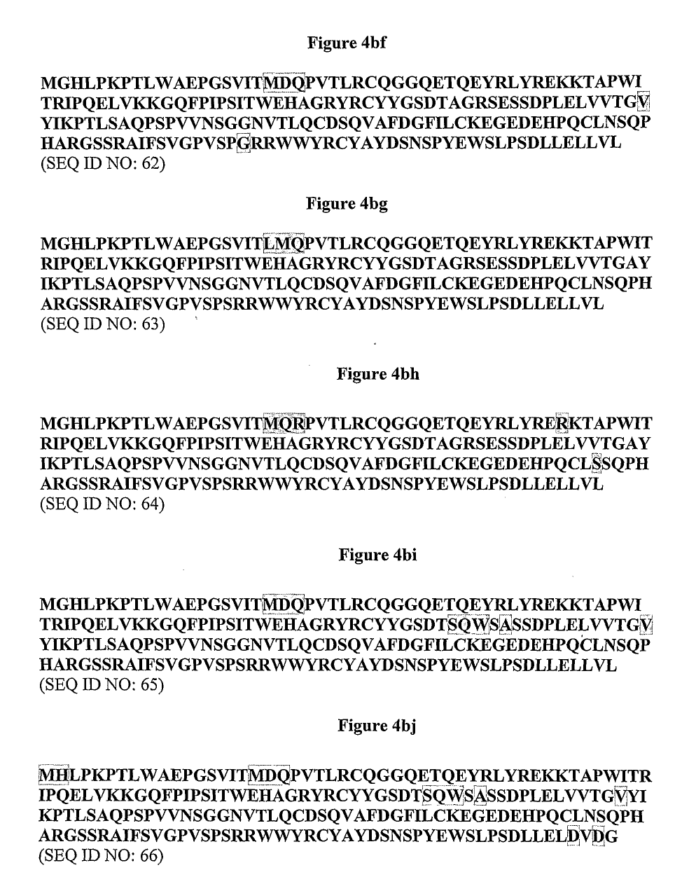

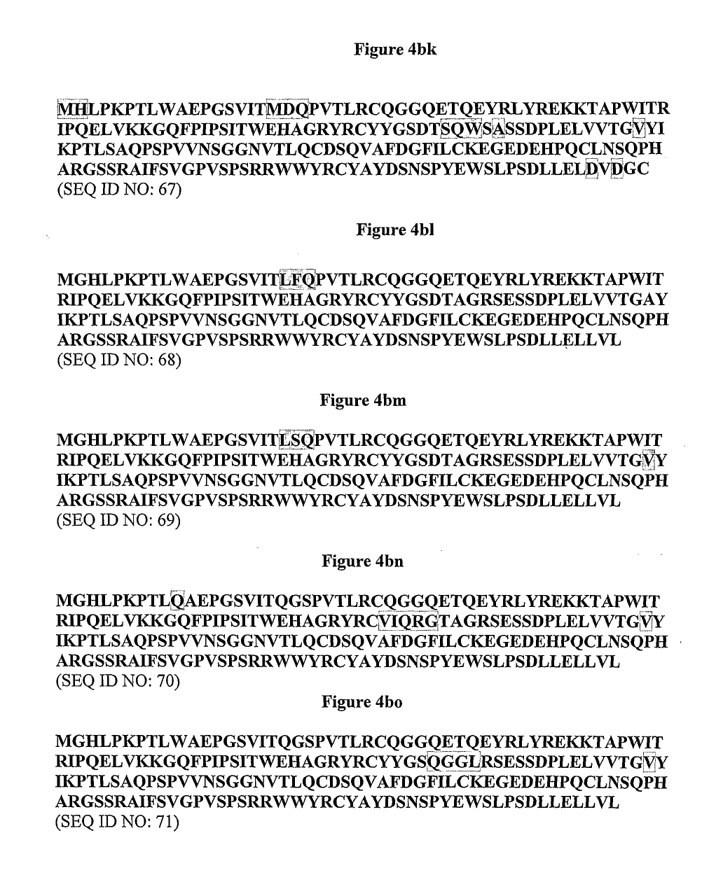

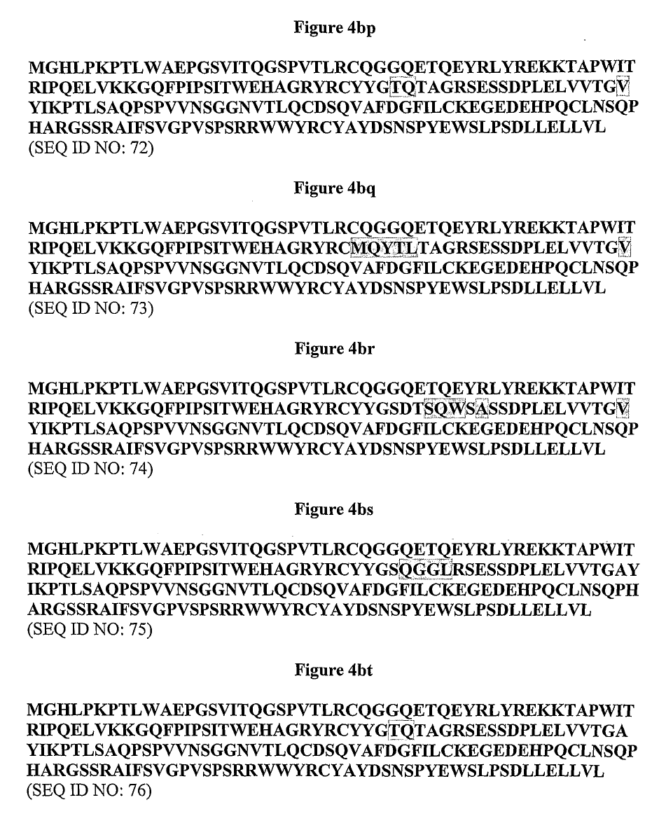

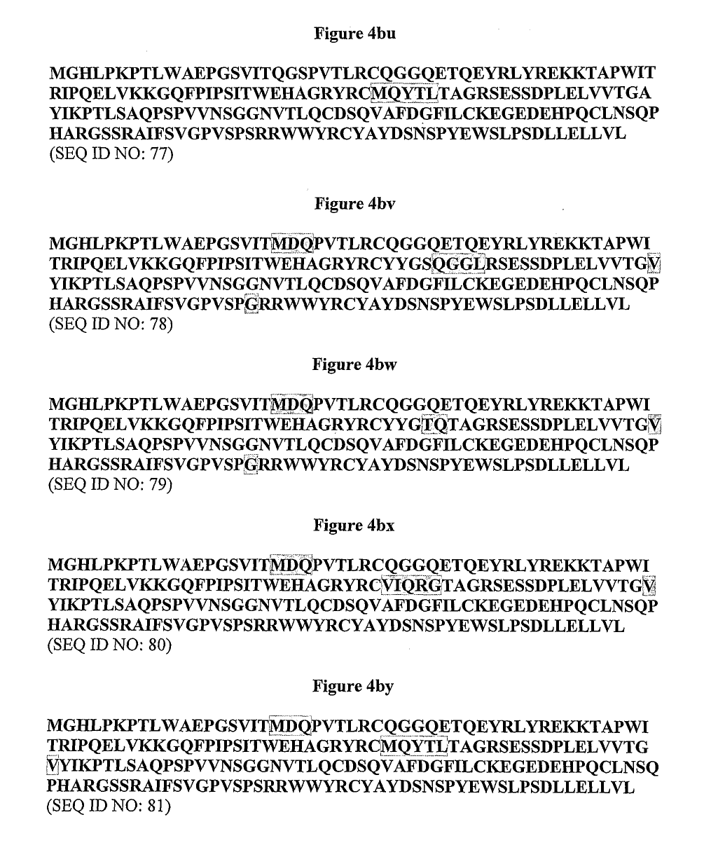

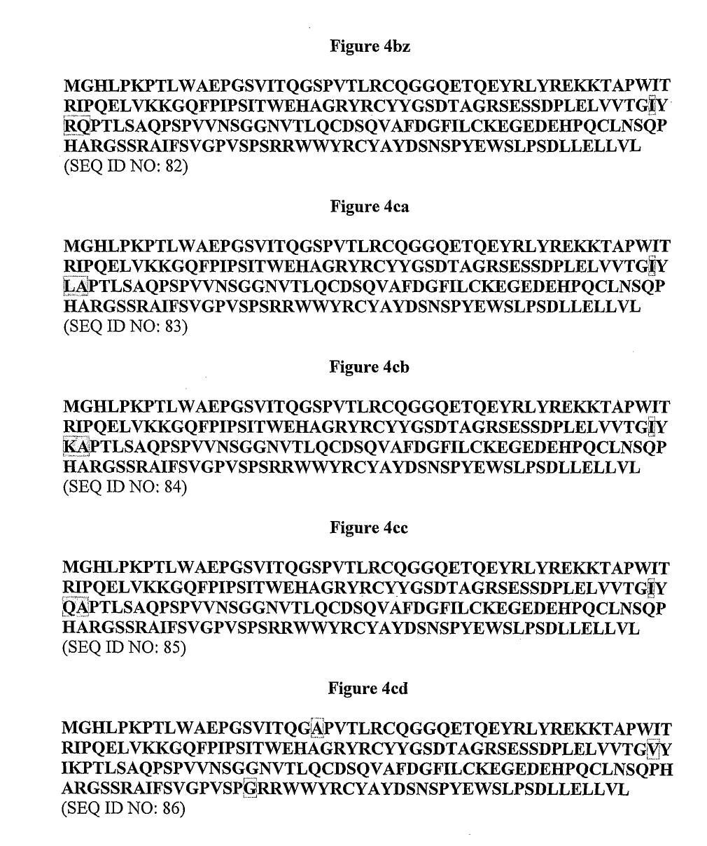

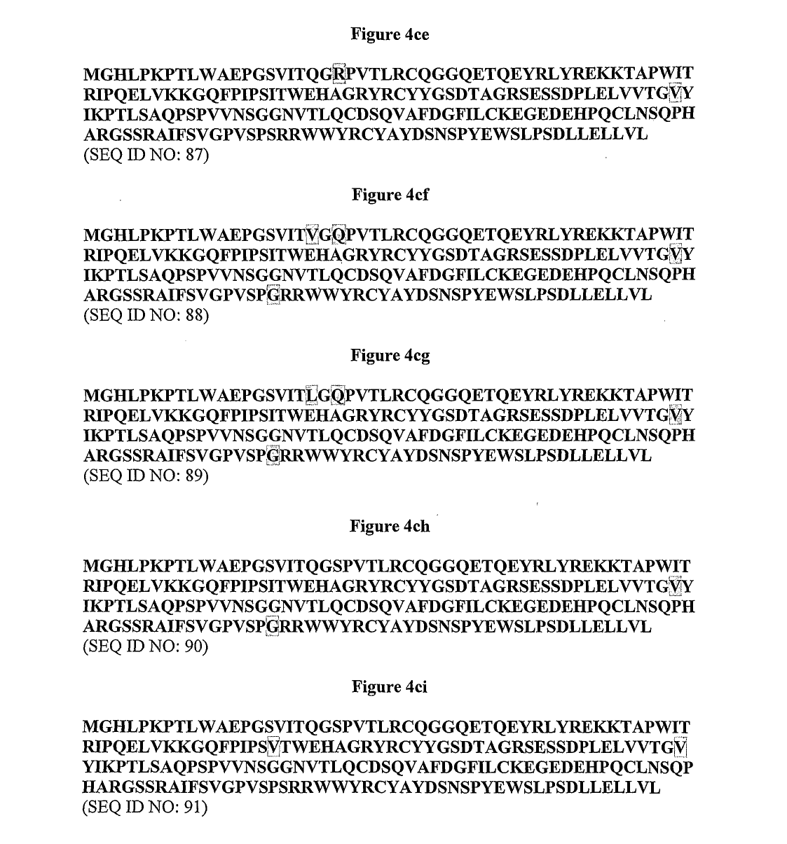

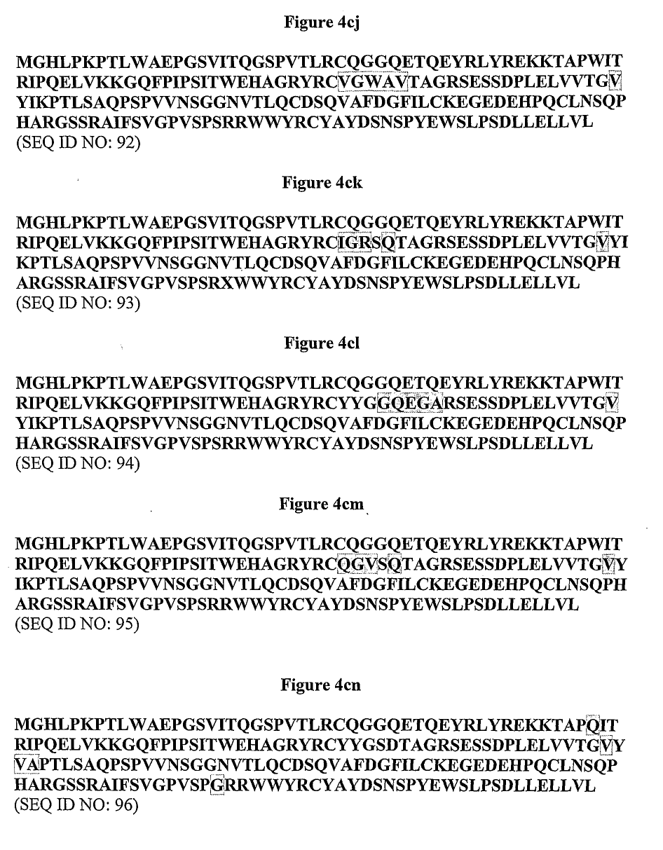

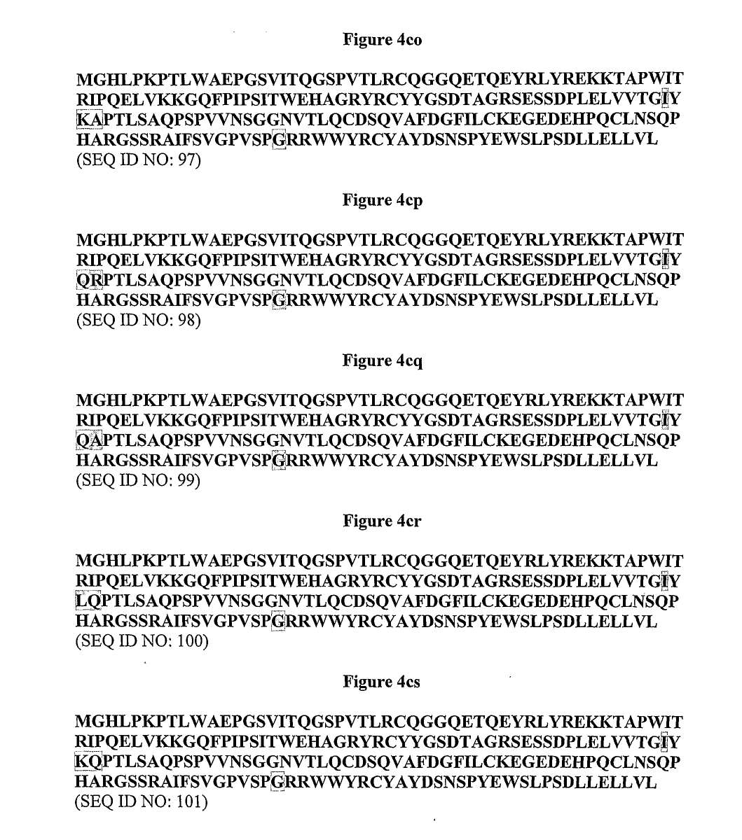

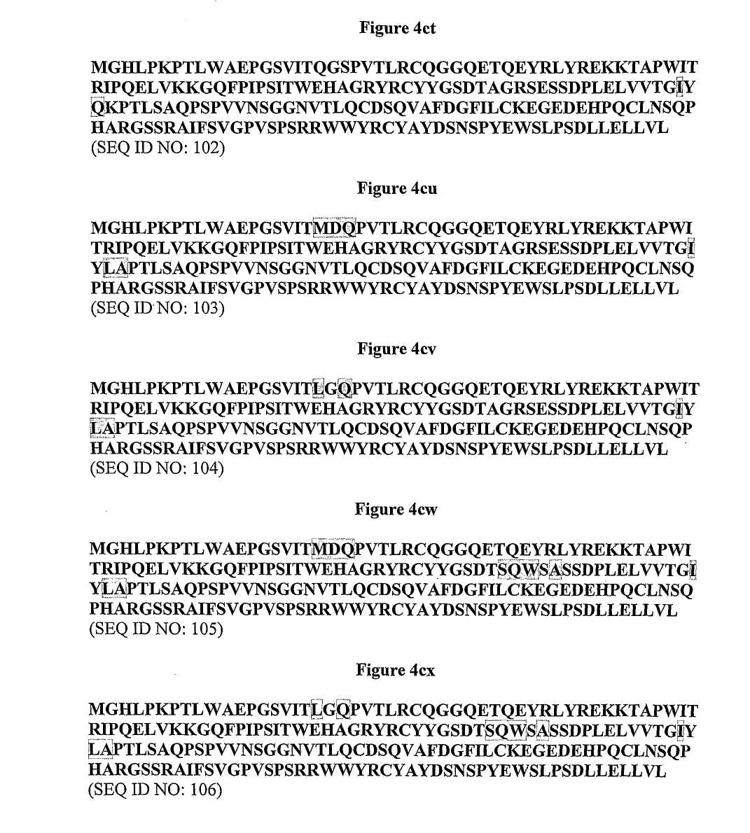

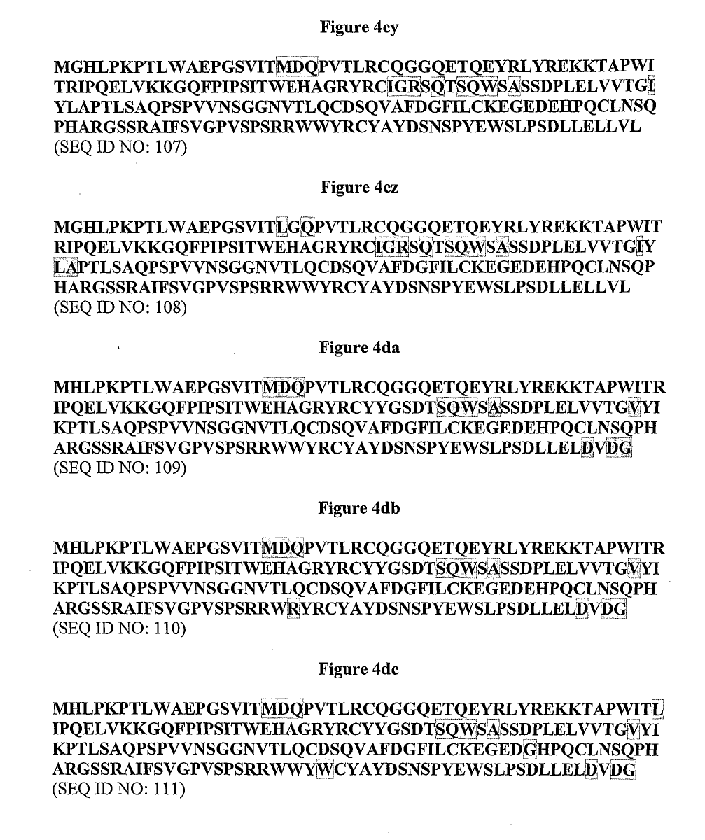

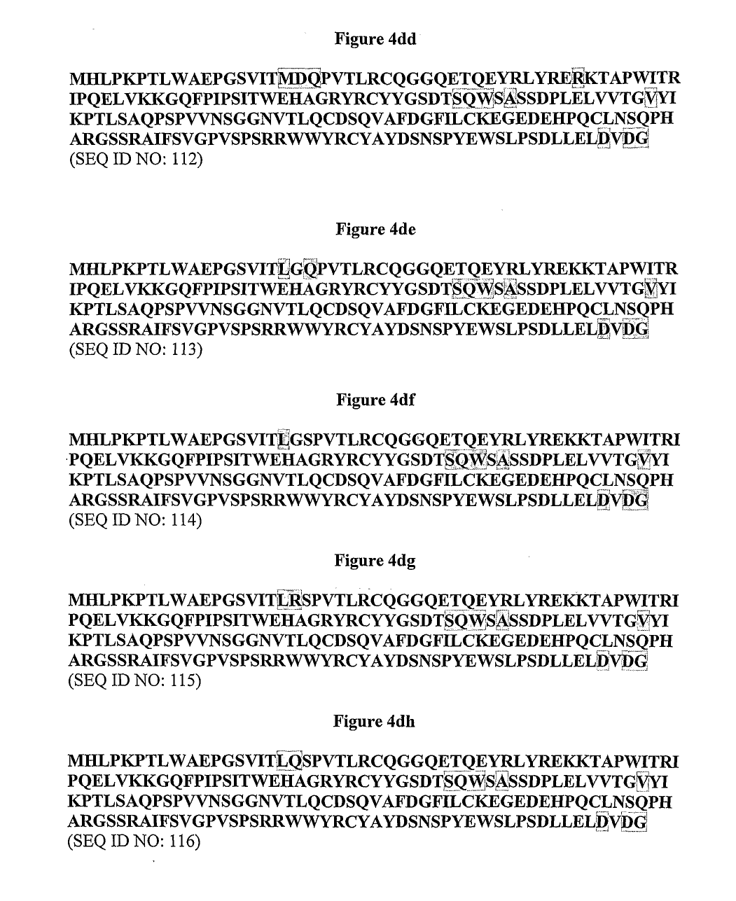

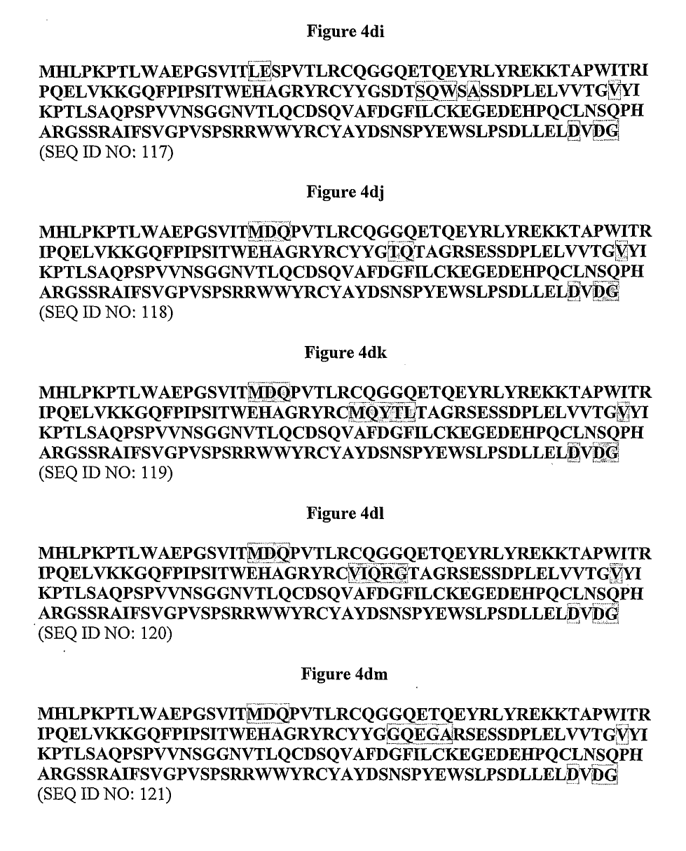

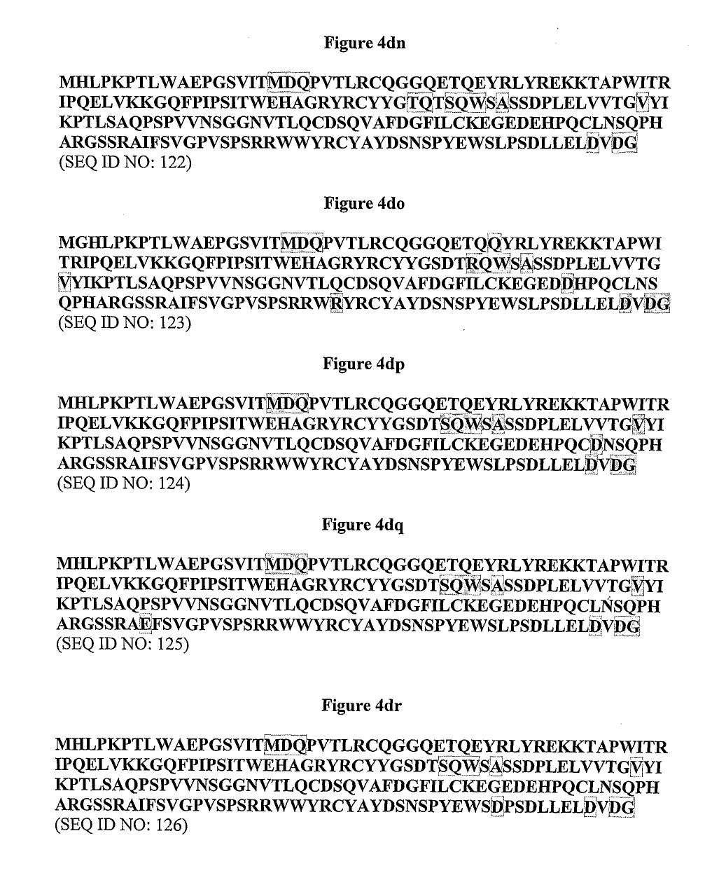

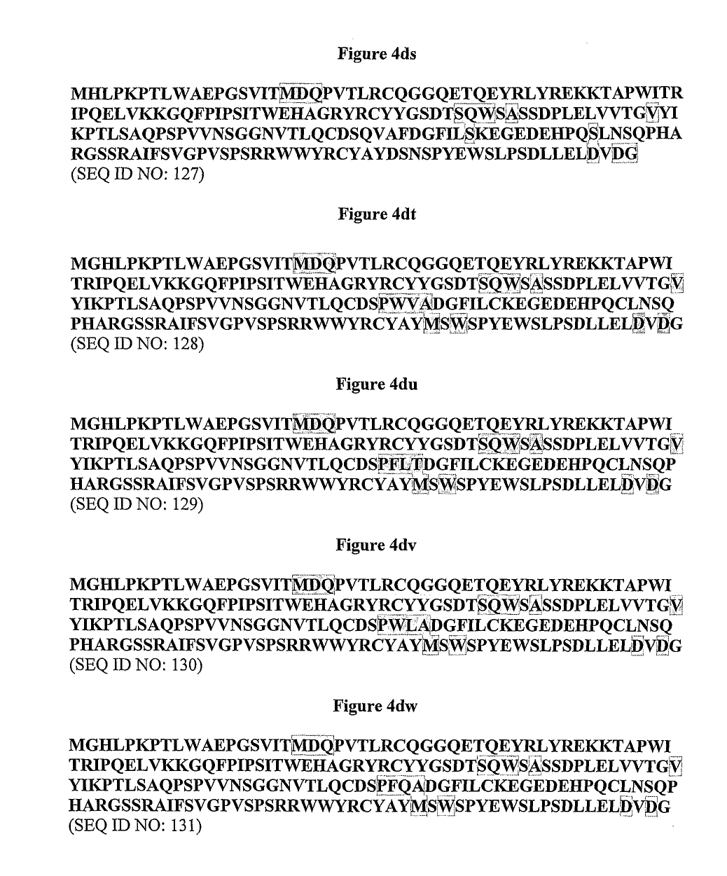

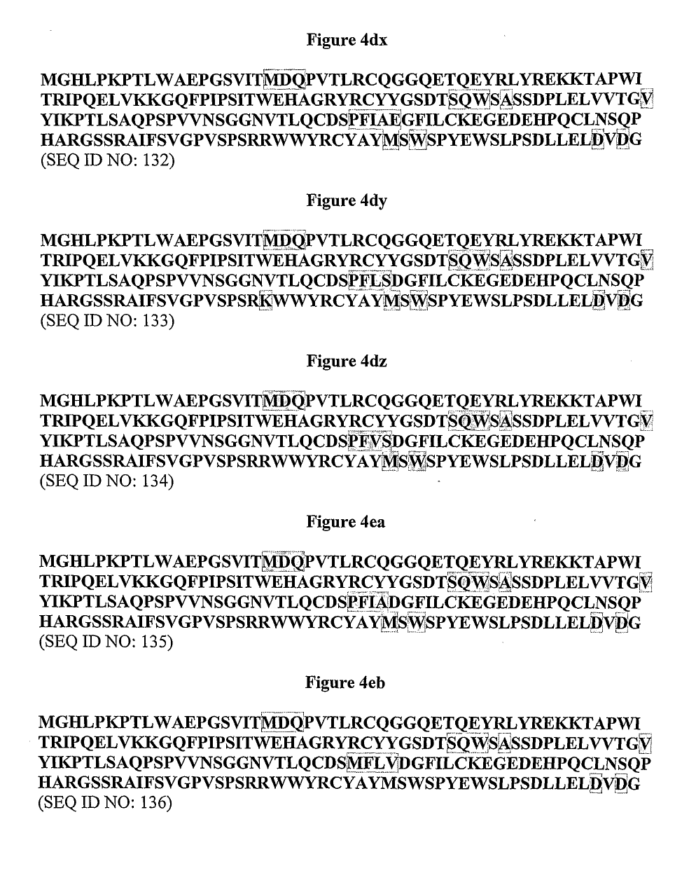

Preferably, such solubility enhancing mutations are in within the C-terminal 6 amino acids of the ILT-like segment(s) of the polypeptide monomers or dimers of the invention. The inclusion of one or both of mutations in said ILT-like segment(s) corresponding to 196D and/or 198D using the numbering of SEQ ID NO: 3 in these segments(s) provide preferred means of increasing the solubility of the polypeptide monomers or dimers of the invention relative to the corresponding polypeptide monomers and dimers lacking said mutation(s). The exemplary ILT-like segments of the invention provided in FIGS. 4a-4bd, 4bj-4bk and 4da-4eb (SEQ ID NOs: 6 to 60, 66-67, and 109-136 respectively) all incorporate both the 196L→D and 198L→D mutations.

### Polypeptide Monomers and Dimers of the Invention Comprising a C-Terminal Reactive Site

The polypeptide monomers and dimers of the invention may be used in multimeric forms or in association with other moieties. In this regard it is desirable to produce polypeptide monomers and dimers of the invention which comprising a means of attaching other moieties thereto.

Therefore, one embodiment is provided by a polypeptide monomer or dimer of the invention which comprises a C-terminal reactive site for covalent attachment of a desired moiety. This reactive site may be a cysteine residue.

As will be known to those skilled in the art there are many reactive chemistries which are suitable for this purpose. These include, but are not limited to, cysteine residues, hexahistidine peptides, biotin and chemically reactive groups. The presence of such reactive chemistries may also facilitate purification of the polypeptide dimers.

### PEGylated Polypeptide Monomers and Dimers of the Invention

In one particular embodiment a polypeptide monomer or dimer of the invention is associated with at least one polyalkylene glycol chain(s). This association may be caused in a number of ways known to those skilled in the art. In a preferred embodiment the polyalkylene chain(s) is/are covalently linked to the polypeptide monomer or dimer. In a further embodiment the polyethylene glycol chains of the present aspect of the invention comprise at least two polyethylene repeating units.

Multivalent Complexes Comprising the Polypeptide Monomers and/or Dimers of the Invention

One aspect of the invention provides a multivalent complex comprising at least two polypeptide monomers and/or dimers of the invention. In one embodiment of this aspect the polypeptide monomers or dimers are linked by a non-peptidic polymer chain or a peptidic linker sequence. A further embodiment of the present aspect is provided by multivalent complexes which contain two or four polypeptides selected from the polypeptide monomers or dimers of the invention.

Preferably, such multivalent complexes of the invention are water soluble, so the linker moiety should be selected accordingly. Furthermore, it is preferable that the linker moiety should be capable of attachment to defined positions on the polypeptide dimers, so that the structural diversity of the complexes formed is minimised.

A further embodiment of the present aspect is provided by a multivalent complex of the invention wherein the polymer chain or peptidic linker sequence extends between amino acid residues of each polypeptide monomer and/or dimer which are not located in the Class I pMHC binding domain of the polypeptide monomer or dimers.

Since the complexes of the invention may be for use in medicine, the linker moieties should be chosen with due regard to their pharmaceutical suitability, for example their immunogenicity.

Examples of linker moieties which fulfil the above desirable criteria are known in the art, for example the art of linking antibody fragments.

There are two classes of linker that are preferred for use in the production of multivalent complexes of the present invention. A multivalent complex of the invention in which the polypeptide monomers and/or dimers are linked by a polyalkylene glycol chain or a peptidic linker derived from a human multimerisation domain provide certain embodiments of the invention.

Suitable hydrophilic polymers include, but are not limited to, polyalkylene glycols. The most commonly used polymers of this class are based on polyethylene glycol or PEG, the structure of which is shown below.

HOCH₂CH₂—O(CH₂CH₂O)ₙ—CH₂CH₂OH

Wherein n is greater than two.

However, others are based on other suitable, optionally substituted, polyalkylene glycols include polypropylene glycol, and copolymers of ethylene glycol and propylene glycol.

Such polymers may be used to treat or conjugate therapeutic agents, particularly polypeptide or protein therapeutics, to achieve beneficial changes to the pharmacokinetic (PK) profile of the therapeutic, for example reduced renal clearance, improved plasma half-life, reduced immunogenicity, and improved solubility. Such improvements in the PK profile of the PEG-therapeutic conjugate are believe to result from the PEG molecule or molecules forming a ‘shell’ around the therapeutic which sterically hinders the reaction with the immune system and reduces proteolytic degradation. (Casey et al, (2000) Tumor Targetting 4 235-244) The size of the hydrophilic polymer used may in particular be selected on the basis of the intended therapeutic use of the polypeptide monomers, dimers or multivalent complexes of the invention. There are numerous review papers and books that detail the use of PEG and similar molecules in pharmaceutical formulations. For example, see (Harris (1992) Polyethylene Glycol Chemistry—Biotechnical and Biomedical Applications, Plenum, New York, N.Y.) or (Harris & Zalipsky (1997) Chemistry and Biological Applications of Polyethylene Glycol ACS Books, Washington, D.C.).

The polymer used can have a linear or branched conformation. Branched PEG molecules, or derivatives thereof, can be induced by the addition of branching moieties including glycerol and glycerol oligomers, pentaerythritol, sorbitol and lysine.

Usually, the polymer will have a chemically reactive group or groups in its structure, for example at one or both termini, and/or on branches from the backbone, to enable the polymer to link to target sites in the polypeptide monomer dimer of the invention. This chemically reactive group or groups may be attached directly to the hydrophilic polymer, or there may be a spacer group/moiety between the hydrophilic polymer and the reactive chemistry as shown below:
Reactive chemistry-Hydrophilic polymer-Reactive chemistry
Reactive chemistry-Spacer-Hydrophilic polymer-Spacer-Reactive chemistry

The spacer used in the formation of constructs of the type outlined above may be any organic moiety that is a non-reactive, chemically stable, chain, Such spacers include, by are not limited to the following:

—(CH₂)ₙ—

wherein n=2 to 5

—(CH₂)₃NHCO(CH₂)₂

A multivalent complex of the invention in which a divalent alkylene spacer radical is located between the polyalkylene glycol chain and its point of attachment to a polypeptide monomer or dimer of the complex provides a further embodiment of the present aspect.

A multivalent complex of the invention in which the polyalkylene glycol chain comprises at least two polyethylene glycol repeating units provides a further embodiment of the present aspect.

There are a number of commercial suppliers of hydrophilic polymers linked, directly or via a spacer, to reactive chemistries that may be of use in the present invention. These suppliers include Nektar Therapeutics (CA, USA), NOF Corporation (Japan), Sunbio (South Korea) and Enzon Pharmaceuticals (NJ, USA).

Commercially available hydrophilic polymers linked, directly or via a spacer, to reactive chemistries that may be of use in the present invention include, but are not limited to, the following:

| PEG linker | | Catalogue |
|---|---|---|
| Description | Source of PEG | Number |
| Attachment to single polypeptide | | |
| monomer or dimer of the invention | | |
| 5K linear (Maleimide) | Nektar | 2D2MOHO1 |
| 20K linear (Maleimide) | Nektar | 2D2MOPO1 |
| 20K linear (Maleimide) | NOF Corporation | SUNBRIGHT |
| | | ME-200MA |
| 20K branched (Maleimide) | NOF Corporation | SUNBRIGHT |
| | | GL2-200MA |
| 30K linear (Maleimide) | NOF Corporation | SUNBRIGHT |
| | | ME-300MA |
| 40K branched PEG (Maleimide) | Nektar | 2D3XOTO1 |
| 5K-NP linear (for Lys attachment) | NOF Corporation | SUNBRIGHT |
| | | MENP-50H |
| 10K-NP linear (for Lys attachment) | NOF Corporation | SUNBRIGHT |
| | | MENP-10T |
| 20K-NP linear (for Lys attachment) | NOF Corporation | SUNBRIGHT |
| | | MENP-20T |
| Linkers for dimers of the | | |
| polypeptide monomers or dimers | | |
| of the invention | | |
| 3.4K linear (Maleimide) | Nektar | 2D2DOFO2 |
| 5K forked (Maleimide) | Nektar | 2D2DOHOF |
| 10K linear (with orthopyridyl ds- | Sunbio | |
| linkers in place of Maleimide) | | |
| 20K forked (Maleimide) | Nektar | 2D2DOPOF |
| 20K linear (Maleimide) | NOF Corporation | |
| 40K forked (Maleimide) | Nektar | 2D3XOTOF |
| Linkers for higher order | | |
| multimers of the polypeptide | | |
| monomers or dimers of the | | |
| invention | | |
| 15K, 3 arms, Mal₃ (for trimer) | Nektar | OJOONO3 |
| 20K, 4 arms, Mal₄ (for tetramer) | Nektar | OJOOPO4 |
| 40 K, 8 arms, Mal₈ (for octamer) | Nektar | OJOOTO8 |

A wide variety of coupling chemistries can be used to couple polymer molecules to protein and peptide therapeutics. The choice of the most appropriate coupling chemistry is largely dependant on the desired coupling site. For example, the following coupling chemistries have been used attached to one or more of the termini of PEG molecules (Source: Nektar Molecular Engineering Catalogue 2003):
N-maleimide
Vinyl sulfone
Benzotriazole carbonate
Succinimidyl proprionate
Succinimidyl butanoate
Thio-ester
Acetaldehydes
Acrylates
Biotin
Primary amines

As stated above non-PEG based polymers also provide suitable linkers for multimerising the polypeptide monomers and/or dimers of the present invention. For example, moieties containing maleimide termini linked by aliphatic chains such as BMH and BMOE (Pierce, products Nos. 22330 and 22323) can be used.

Peptidic linkers are the other class of multivalent complex linkers. These linkers are comprised of chains of amino acids, and function to produce simple linkers or multimerisation domains onto which the polypeptide monomers or dimers of the present invention can be attached. The biotin/streptavidin system has previously been used to produce tetramers of TCRs and pMHC molecules (see WO 99/60119) for in-vitro binding studies. However, streptavidin is a microbially-derived polypeptide and as such not ideally suited to use in a therapeutic.

Multivalent complexes of the invention in which the polypeptide monomers and/or dimers are linked by a peptidic linker derived from a human multimerisation domain provide one embodiment of the present aspect. There are a number of human proteins that contain a multimerisation domain that could be used in the production of multivalent complexes of the polypeptide monomers and/or dimers of the invention. For example, the tetramerisation domain of p53 has been utilised to produce tetramers of scFv antibody fragments which exhibited increased serum persistence and significantly reduced off-rate compared to the monomeric scFv fragment. (Willuda et al. (2001) J. Biol. Chem. 276 (17) 14385-14392) Haemoglobin also has a tetramerisation domain that could potentially be used for this kind of application.

A further embodiment of the present aspect is provided by multivalent complexes of the invention associated with a therapeutic agent.

A further aspect is provided by a polypeptide monomer or dimer, or a multivalent complex of the invention which is soluble.

### Diagnostic and Therapeutic Use

In one aspect the polypeptide monomers and/or dimers of the invention or multivalent complexes thereof may be labelled with an imaging compound, for example a label that is suitable for diagnostic purposes. Such labelled polypeptide dimers are useful in a method for detecting target pMHC molecules which method comprises contacting the pMHC with a polypeptide dimer of the invention or a multivalent complex thereof bind to the pMHC; and detecting said binding. In tetrameric complexes formed for example, using biotinylated polypeptide dimer molecules, fluorescent streptavidin can be used to provide a detectable label. Such a fluorescently-labelled tetramer is suitable for use in FACS analysis, for example to detect antigen presenting cells.

In a further aspect a polypeptide monomer or dimer of the present invention may alternatively or additionally be associated with (e.g. covalently or otherwise linked to) a therapeutic agent or detectable label.

In a specific embodiment of the invention said polypeptide monomer or dimer may be covalently, linked to a therapeutic agent or detectable label. For example, the therapeutic agent may be linked to an Fc-like segment of said polypeptide monomer or dimer.

In certain embodiments of the present aspect said therapeutic agent is an immune effector molecule. The said immune effector molecule may be a cytokine.

As is known to those skilled in the art there are a number of cytokines which generally act to “suppress” immune responses. Polypeptide monomers or dimers of the invention associated with such immuno-suppressive cytokines form preferred embodiments of the invention. Polypeptide monomers or dimers of the invention associated with IL-4, IL-10 or IL-13 or a phentoypically silent variant or fragment of these cytokines provide specific embodiments of the present invention.

A multivalent complex of a polypeptide monomer or dimer of the invention may have enhanced binding capability for a given pMHC compared to the corresponding non-multimerised polypeptide monomers or dimers of the invention. Thus, the multivalent complexes according to the invention are particularly useful for tracking or targeting cells presenting particular antigens in vitro or in vivo, and are also useful as intermediates for the production of further multivalent complexes having such uses.

Pharmaceutical compositions comprising a polypeptide monomer or dimer of the invention, or a multivalent complex thereof, together with a pharmaceutically acceptable carrier therefore provide a further aspect of the invention. A related embodiment is provided by the therapeutic use of a polypeptide monomer or dimer of the invention or a multivalent complex thereof.

Pharmaceutical compositions comprising a polypeptide monomer or dimer of the invention or a multivalent complex thereof associated with a therapeutic agent together with a pharmaceutically acceptable carrier therefore provide a further aspect of the invention. A related embodiment is provided by the therapeutic use of a polypeptide monomer or dimer of the invention or a multivalent complex thereof associated with a therapeutic agent.

Autoimmune diseases which may be amenable to treatment by the compositions of the present invention include, but are not limited to, diseases such as Diabetes, Goodpasture's syndrome, Multiple sclerosis, Psoriasis, Rheumatoid arthritis, Myositis, Ankylosing spondylitis, Artery aneurysms in acute Kawasaki disease, Hashimoto's disease and Crohn's disease.

Other diseases which may also be amenable to treatment by the compositions of the present invention include, but are not limited to, Asthma, Eczema, Allograft rejection, Graft-versus Host Disease, Hepatitis and Cerebral malaria.

Another aspect of the invention is provided by the use of a polypeptide monomer or dimer of the invention or a multivalent complex thereof, optionally associated with a therapeutic agent, in the manufacture of a medicament for the treatment of autoimmune disease. Such autoimmune diseases include, but are not limited to, diseases such as Diabetes, Goodpasture's syndrome, Multiple sclerosis, Psoriasis, Rheumatoid arthritis, Myositis, Ankylosing spondylitis, Artery aneurysms in acute Kawasaki disease, Hashimoto's disease and Crohn's disease. A related embodiment is provided by the use of a polypeptide monomer or dimer of the invention or a multivalent complex thereof, optionally associated with a therapeutic agent, in the manufacture of a medicament for the treatment of Asthma, Eczema, Allograft rejection, Graft-versus Host Disease, Hepatitis and Cerebral malaria. In certain embodiments of the present aspect said medicaments may be adapted for parenteral administration. Suitable parenteral routes of administration include subcutaneous, intradermal or intramuscular routes.

Soluble polypeptide monomers or dimers or multivalent complexes thereof of the invention may be linked to an enzyme capable of converting a prodrug to a drug. This allows the prodrug to be converted to the drug only at the site where it is required (i.e. targeted by the said polypeptide monomer or dimer or multivalent complex thereof).

It is expected that the polypeptide monomers or dimers or multivalent complexes thereof disclosed herein may be used in methods for the diagnosis and treatment of autoimmune disease.

The invention also provides a method of treatment of autoimmune disease comprising administering to a subject suffering such autoimmune disease an effective amount of a polypeptide monomer or dimer of the invention or multivalent complex thereof, optionally associated with a therapeutic agent. Such autoimmune diseases include, but are not limited to, diseases such as Diabetes, Goodpasture's syndrome, Multiple sclerosis, Psoriasis, Rheumatoid arthritis, Myositis, Ankylosing spondylitis, Artery aneurysms in acute Kawasaki disease, Hashimoto's disease and Crohn's disease. A related embodiment is provided by a method of treatment of a method of treatment of Asthma, Eczema, Allograft rejection, Graft-versus Host Disease, Hepatitis and Cerebral malaria comprising administering to a subject suffering such a disease an effective amount of a polypeptide monomer or dimer of the invention or multivalent complex thereof, optionally associated with a therapeutic agent. In a related embodiment the invention provides for the use of a polypeptide monomer or dimer of the invention or multivalent complex thereof, optionally associated with a therapeutic agent, in the preparation of a composition for the treatment of autoimmune disease, or for the treatment of Asthma, Eczema, Allograft rejection, Graft-versus Host Disease, Hepatitis or Cerebral malaria.

Examples 9 and 10 herein describe in-vitro methods suitable for assessing the ability of the polypeptide monomers and dimers of the invention to inhibit cytotoxic T cell activation and T cell-mediated cell lysis respectively.

Therapeutic or imaging polypeptide monomer or dimers of the invention or multivalent complexes thereof in accordance with the invention will usually be supplied as part of a sterile, pharmaceutical composition which will normally include a pharmaceutically acceptable carrier. This pharmaceutical composition may be in any suitable form, (depending upon the desired method of administering it to a patient). It may be provided in unit dosage form, will generally be provided in a sealed container and may be provided as part of a kit. Such a kit would normally (although not necessarily) include instructions for use. It may include a plurality of said unit dosage forms.

The pharmaceutical composition may be adapted for administration by any appropriate route, for example parenteral, transdermal or via inhalation, preferably a parenteral (including subcutaneous, intramuscular, or, most preferably intravenous) route. Such compositions may be prepared by any method known in the art of pharmacy, for example by mixing the active ingredient with the carrier(s) or excipient(s) under sterile conditions.

Dosages of the substances of the present invention can vary between wide limits, depending upon the disease or disorder to be treated, the age and condition of the individual to be treated, etc. and a physician will ultimately determine appropriate dosages to be used.

### Additional Aspects

A polypeptide monomer or dimer or multivalent complex thereof of the present invention may be provided in substantially pure form, or as a purified or isolated preparation. For example, it may be provided in a form which is substantially free of other proteins.

Further embodiments are provided by nucleic acid encoding a polypeptide monomer of the invention, and by nucleic acid encoding two different polypeptide monomers of the invention. Said nucleic acid may be one which has been adapted for high level expression in a host cell. There are a number of companies which offer such nucleic acid optimisation as a service, for example GeneArt AG, Germany Related embodiments include expression vectors incorporating said nucleic acid and cells containing said vectors.

A final aspect is provided by a method of producing a polypeptide monomer or dimer of the invention comprising:
(i) transforming a host cell with a vector of the invention; and
(ii) culturing the transformed cells under conditions suitable for the expression of the polypeptide monomer or dimer; and
(iii) recovering the expressed polypeptide monomer or dimer

Specific embodiments of the present aspect are provided wherein the host cells are selected from Chinese Hamster Ovary (CHO) cells, E. coli cells or yeast cells, for example Pichia pastoris cells. Example 7 herein provides a method for the production of polypeptide dimers of the invention in CHO cells.

Preferred features of each aspect of the invention are as for each of the other aspects mutatis mutandis. The prior art documents mentioned herein are incorporated to the fullest extent permitted by law.

### EXAMPLES

The invention is further described in the following examples, which do not limit the scope of the invention in any way.

Reference is made in the following to the accompanying drawings in which:

FIG. 1a is the full amino acid sequence of a wild type human ILT-2 (SEQ ID No: 1) The highlighted amino acids show residues of this polypeptide which differ from the corresponding residues of isoform 1 of Wild-type human ILT-2. The amino acids of the transmembrane domain are underlined.

FIG. 1b is the full DNA sequence of a wild type human ILT-2 (SEQ ID No: 2) which encodes the amino acid sequence of FIG. 1a. The DNA sequence corresponds to that given NCIMB Nucleotide accession NO: NM_{—}006669.

FIGS. 2a and 2b respectively are the amino acid and DNA sequence of a soluble two domain form of the wild-type ILT-2 sequences provided in FIGS. 1a and 1b. These truncated sequences contain/encode for only extracellular domains D1 and D2 of ILT-2. (SEQ ID No: 3 and SEQ ID NO: 4 respectively)

FIG. 3 is the full DNA sequence inserted into the pGMT7-based vector in order to express the soluble two domain form of the wild-type ILT-2 polypeptide of FIG. 2a. The HindIII and NdeI restriction enzyme recognition sequences are underlined.

FIGS. 4a to 4eb (SEQ ID Nos 6-136) are the amino acid sequences of soluble two domain high affinity ILT-like polypeptides. The residues which have been mutated relative to those of FIG. 2a are highlighted

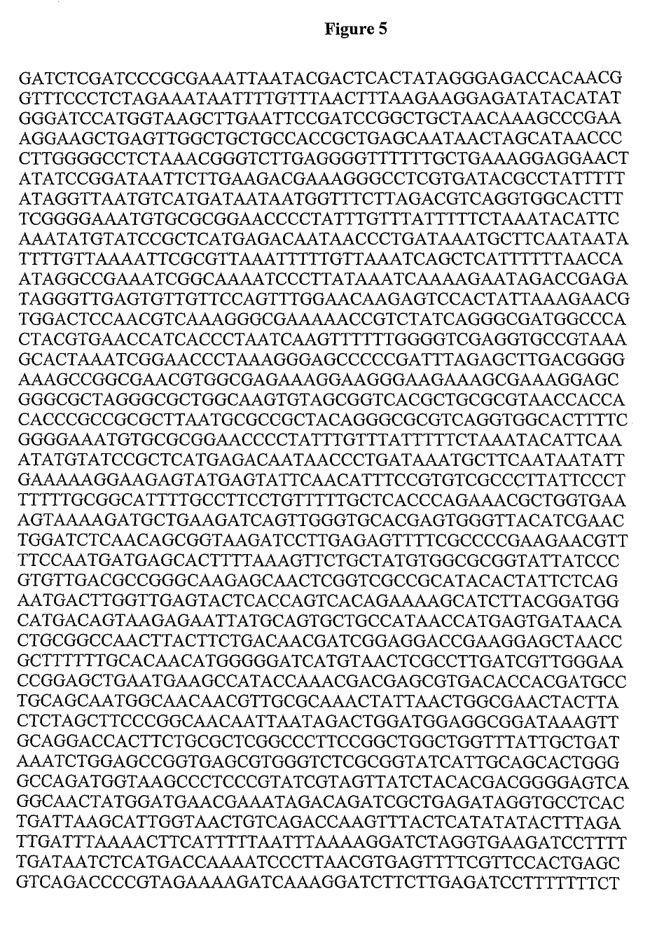

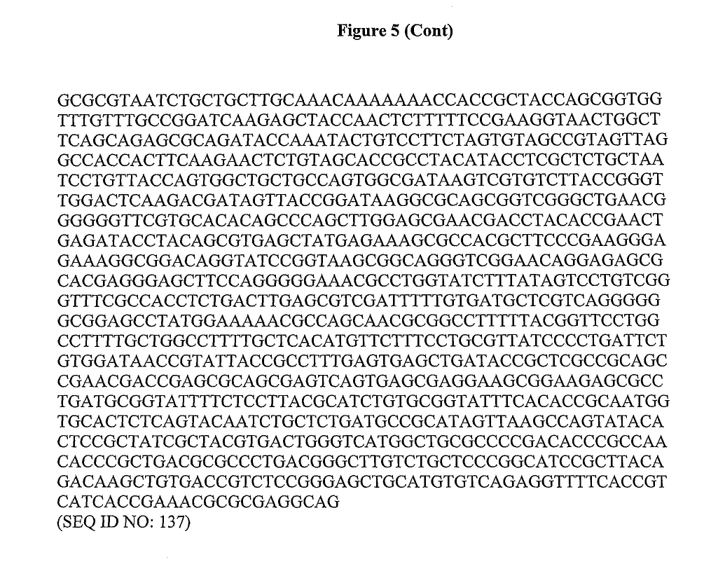

FIG. 5 is the DNA sequence of a pGMT7-derived vector into which DNA encoding the amino acid sequences of the ILT-like segments can be inserted.

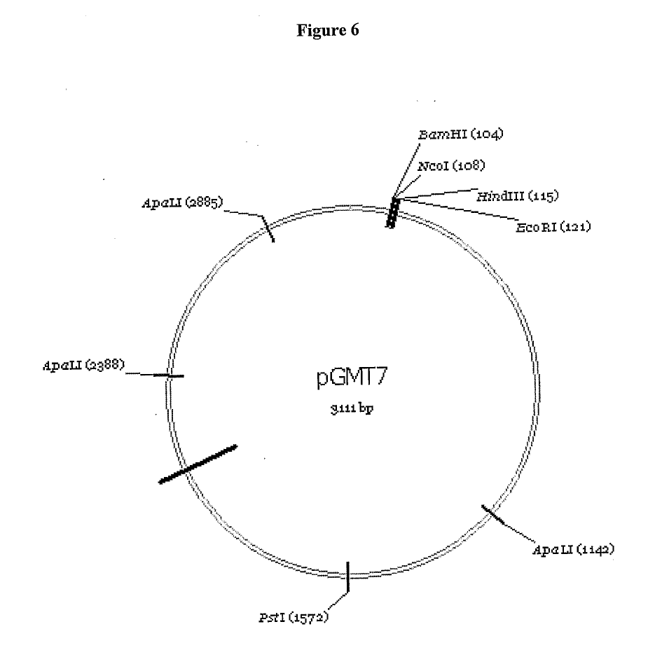

FIG. 6 is the plasmid map of the pGMT7-derived vector detailed in FIG. 5

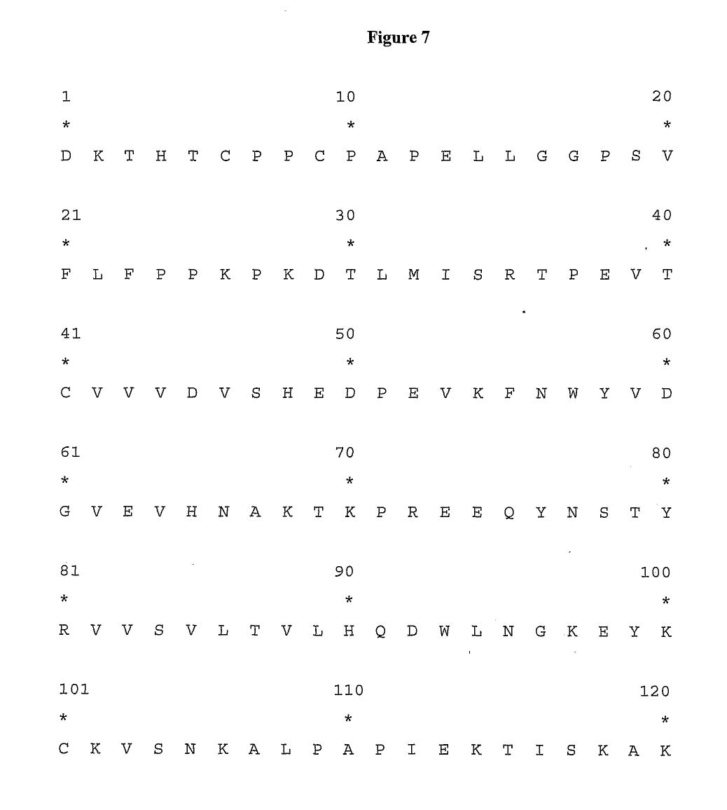

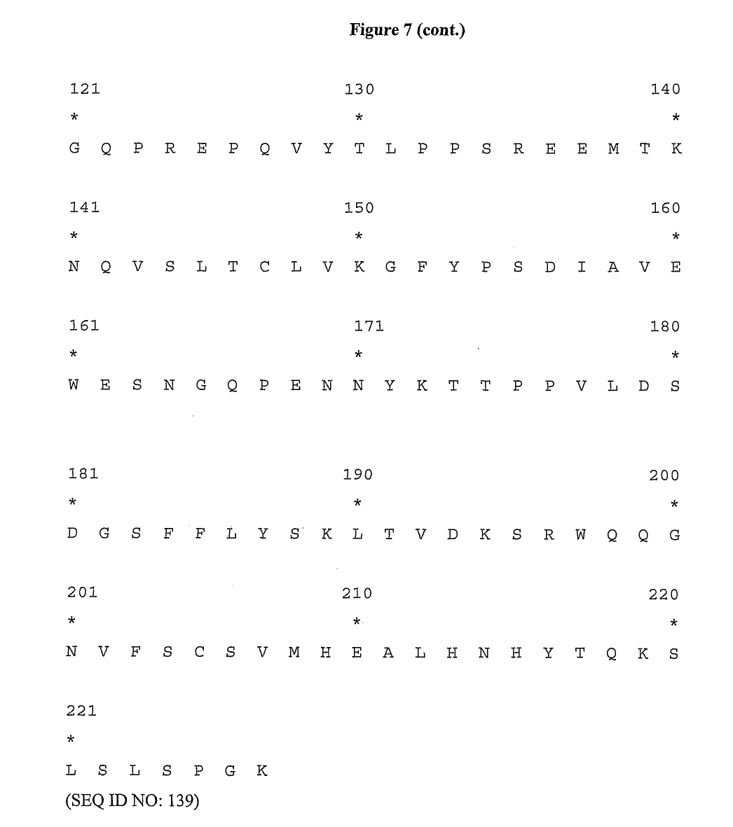

FIG. 7 details the amino acid sequence of the Fc monomer of wild-type human IgG1.

FIG. 8a details the amino acid sequences of a preferred mutated human IgG1 Fc monomer. The amino acid residues in these monomers that have been mutated relative to wild-type human IgG1 are shaded. The mutations which improve PK are in bold and shaded. The mutations which counter ADCC and/or /CDCC are underlined and shaded.

FIG. 8b details the amino acid sequences of another preferred mutated human IgG1 Fc monomer. The amino acid residues in these monomers that have been mutated relative to wild-type human IgG1 are shaded. The mutations which remove native cysteine residues are shaded and itallicised.

FIG. 8c details the amino acid sequences of a further preferred mutated human IgG1 Fc monomer. The amino acid residues in these monomers that have been mutated relative to wild-type human IgG1 are shaded. The mutations which counter ADCC and/or /CDCC are underlined and shaded.

FIG. 8d details the amino acid sequences of a further preferred mutated human IgG1 Fc monomer. The amino acid residues in these monomers that have been mutated relative to wild-type human IgG1 are shaded. The mutations which counter ADCC and/or /CDCC are underlined and shaded.

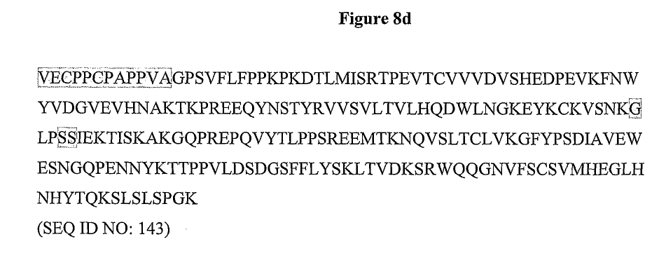

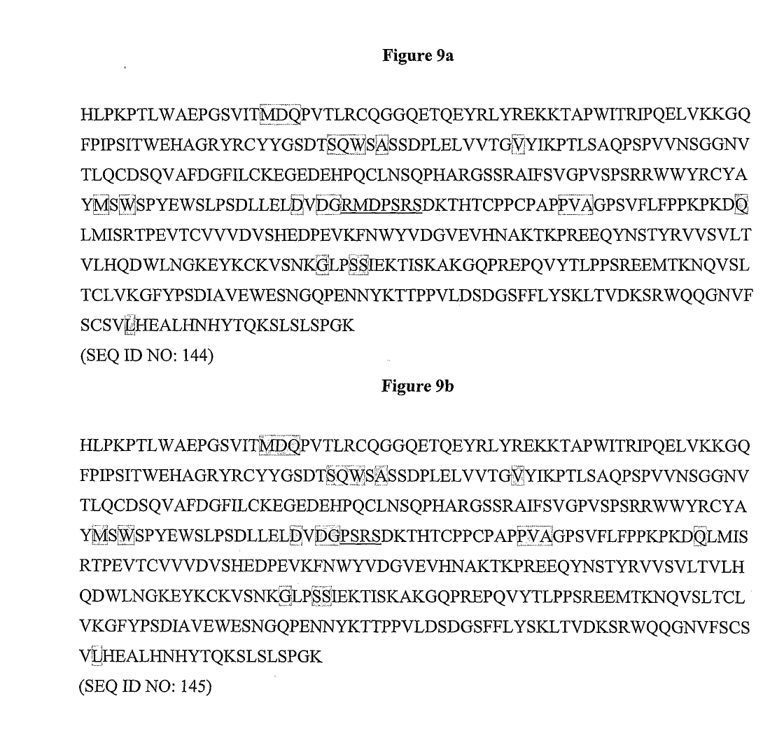

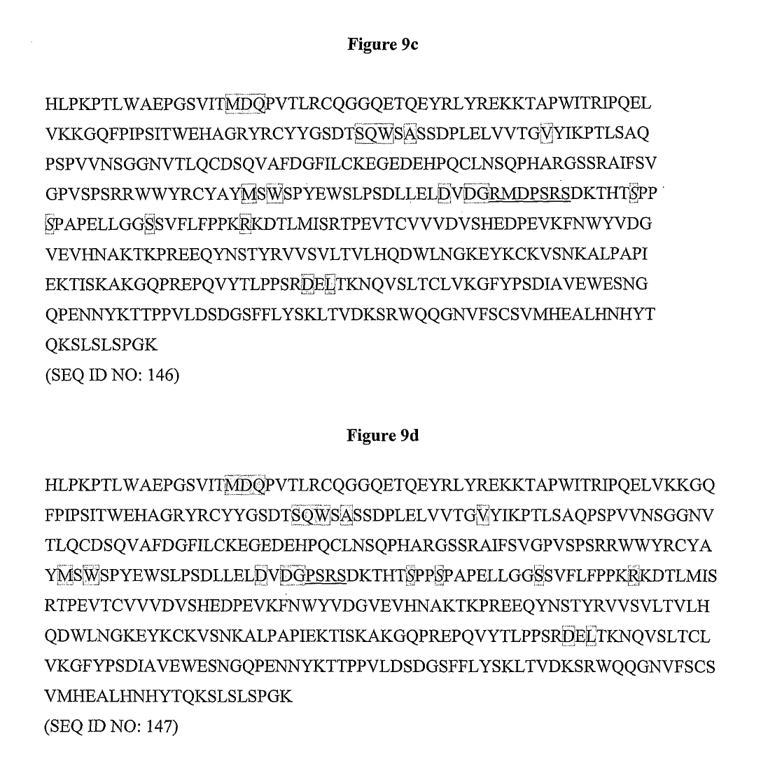

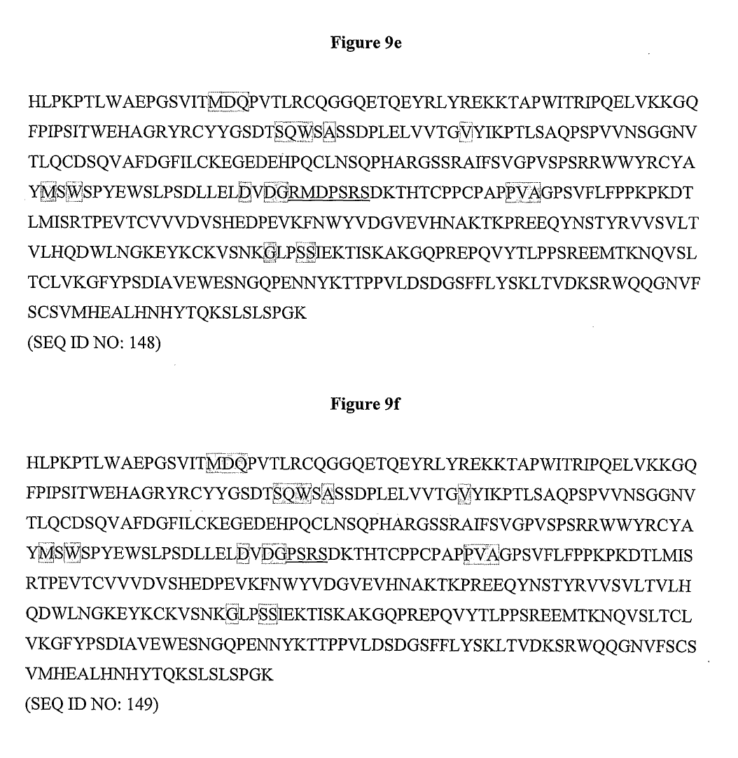

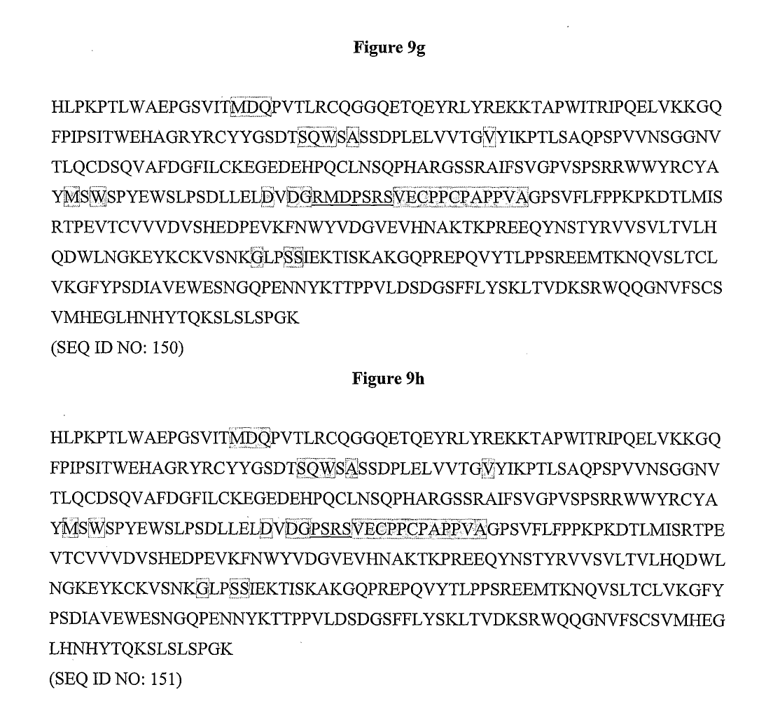

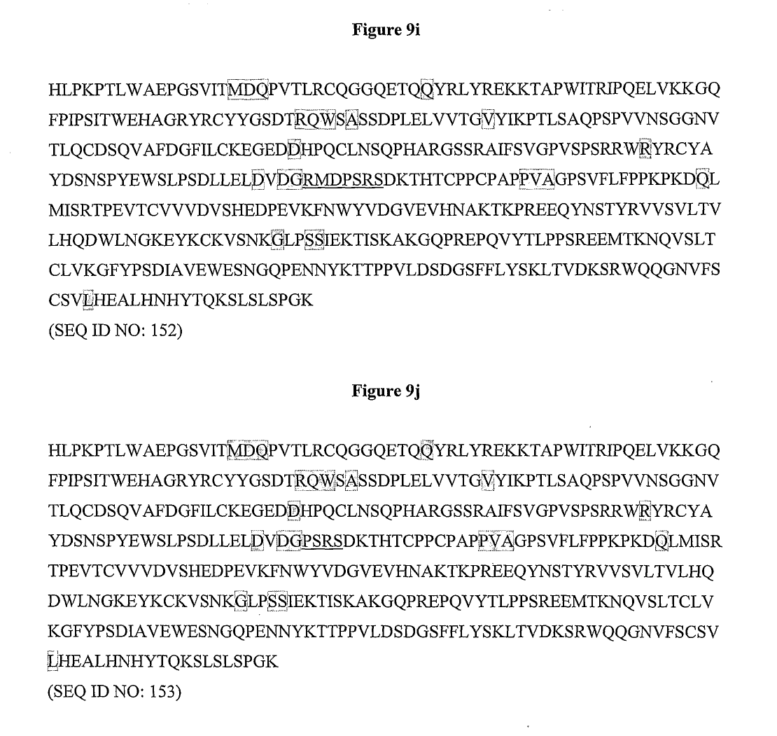

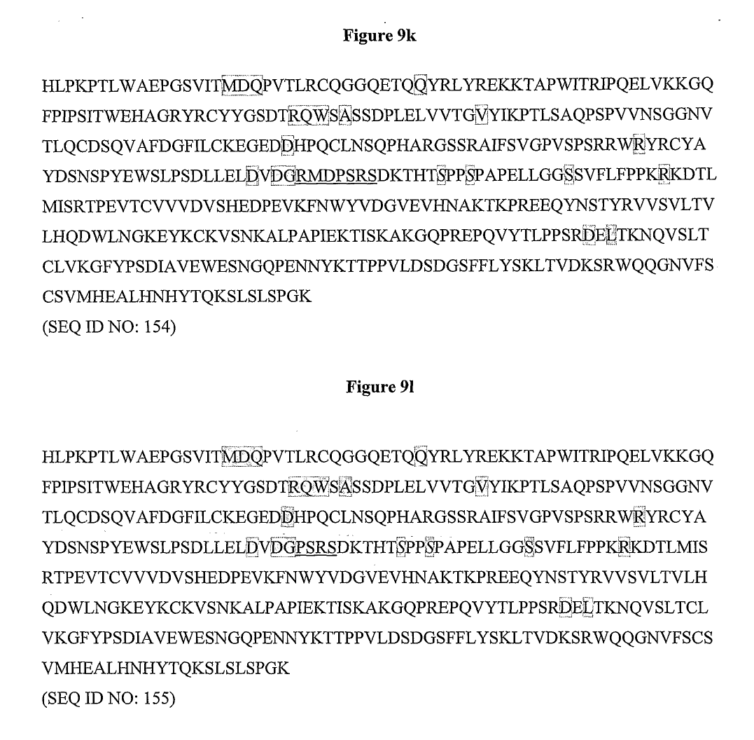

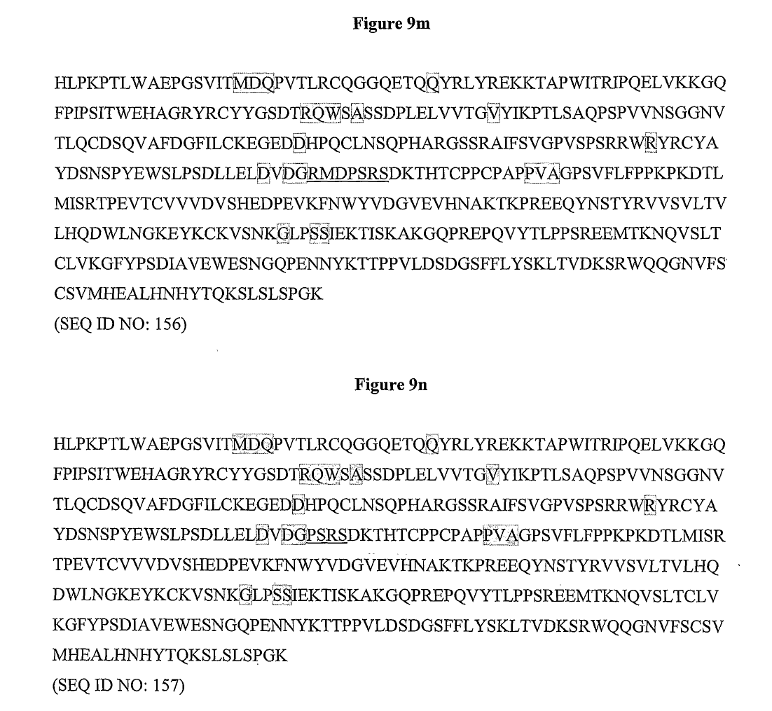

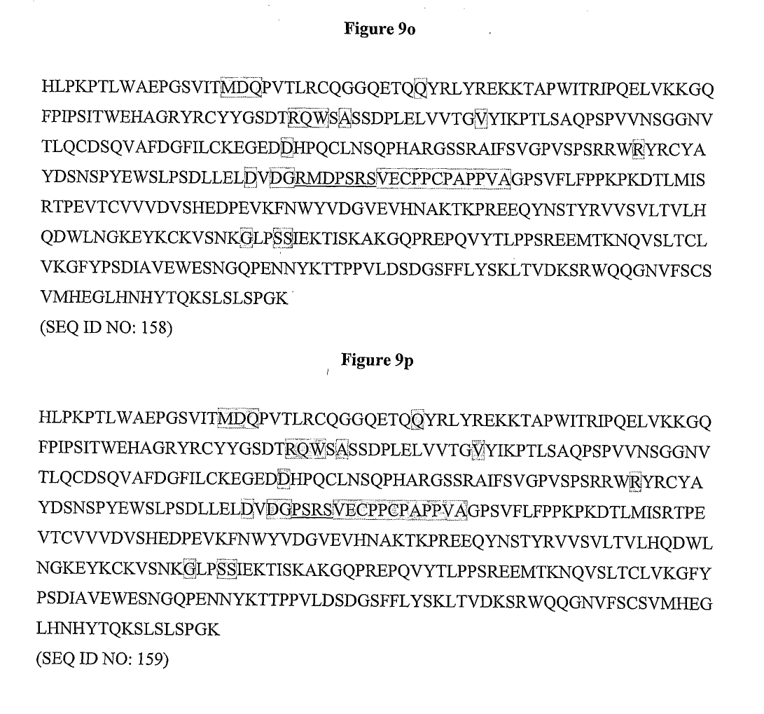

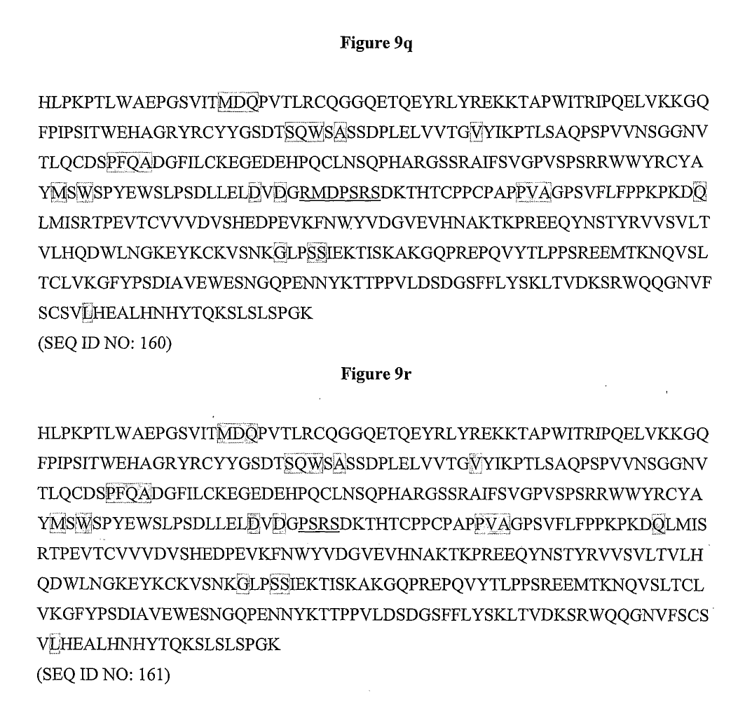

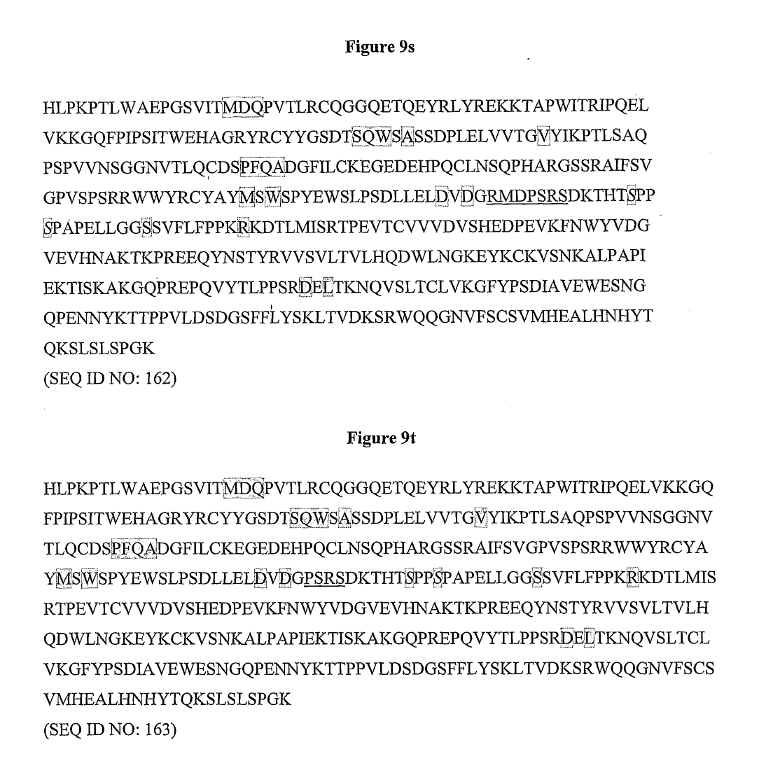

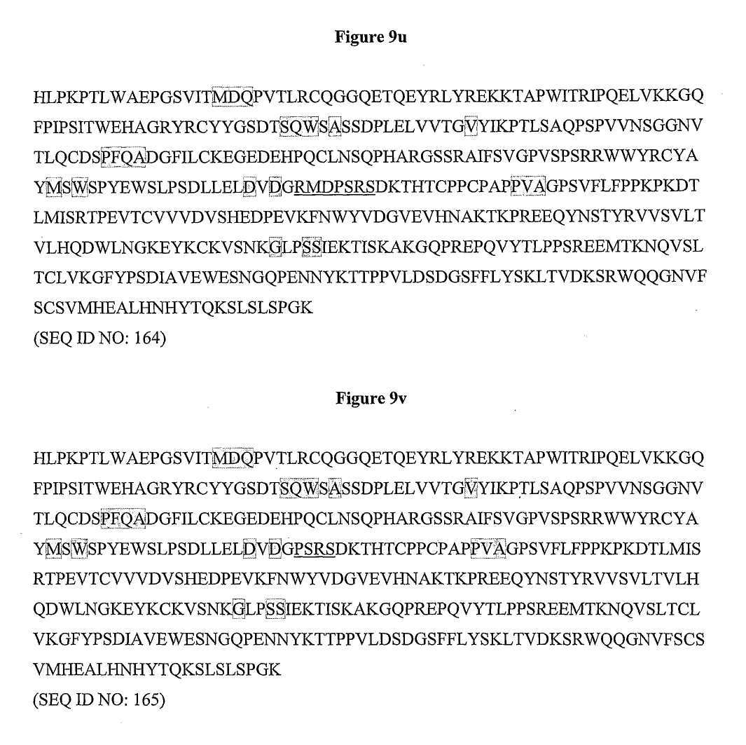

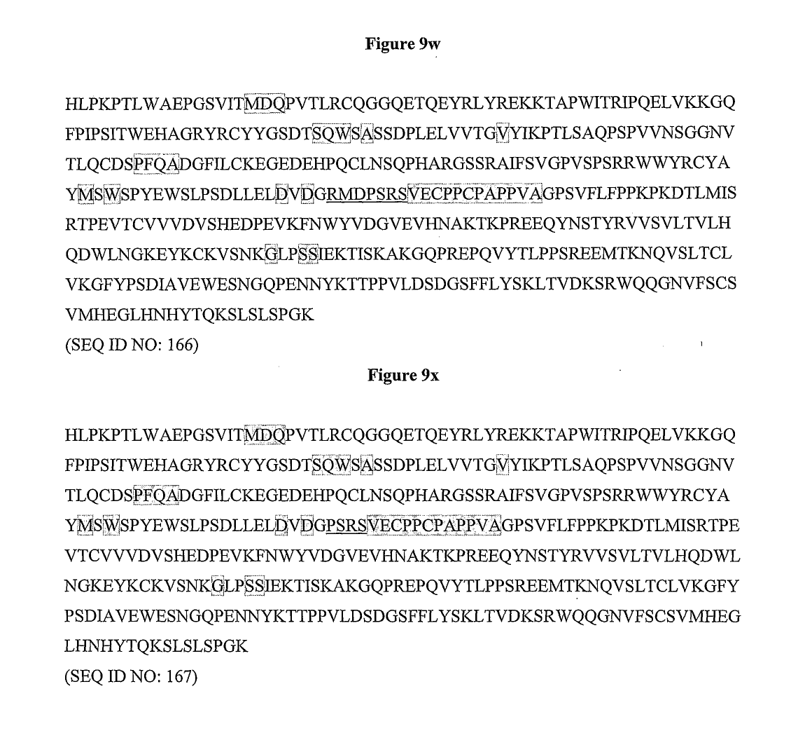

FIGS. 9a to 9x detail the amino acid sequences of preferred high affinity ILT-like Fc fusion polypeptide monomers. The amino acid sequences which are mutated relative to wild-type human ILT-2 and wild-type human IgG1 Fc are shaded. The amino acids within the linker sequences between the ILT-like and Fc-like portions of these fusion polypeptides monomers are underlined.

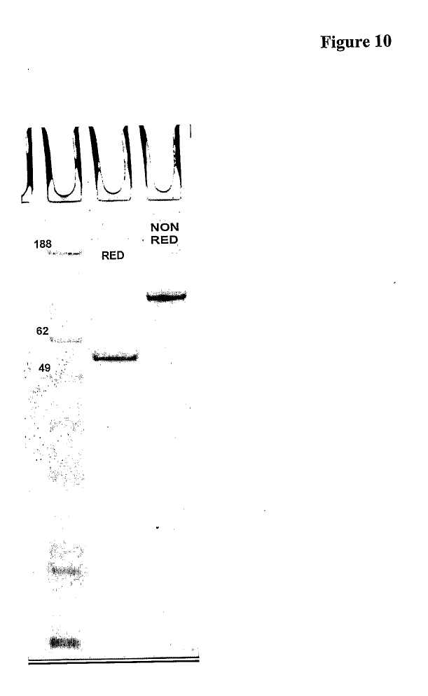

FIG. 10 is a gel of a polypeptide dimer of the invention which comprises two Clone c83 ILT-like segments having the amino acid sequence of SEQ ID NO: 19.

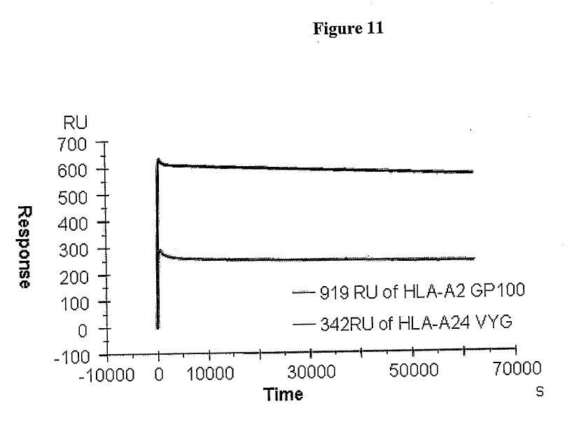

FIG. 11 is a Biacore trace of the interactions of two different Class I pMHC complexes and a polypeptide dimer of the invention which comprises two Clone c83 ILT-like segments having the amino acid sequence of SEQ ID NO: 19.

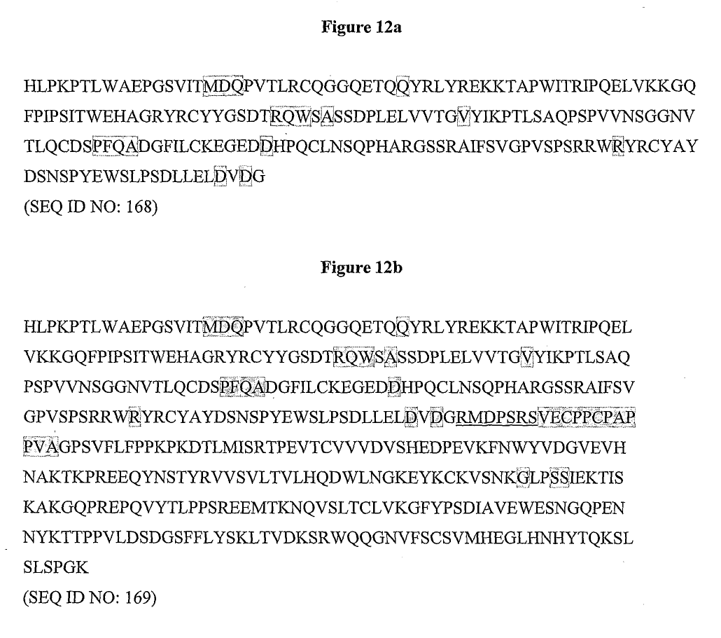

FIG. 12a is the amino acid sequence of a soluble two domain high affinity ILT-like polypeptide. The residues which have been mutated relative to those of FIG. 2a are highlighted. FIG. 12b is the amino acid sequences of a member of the class of polypeptide monomers containing an Fc-like segment and two domain high affinity ILT-like polypeptide of FIG. 12a. The residues in the ILT-like polypeptide which have been mutated relative to those of FIG. 2a are highlighted

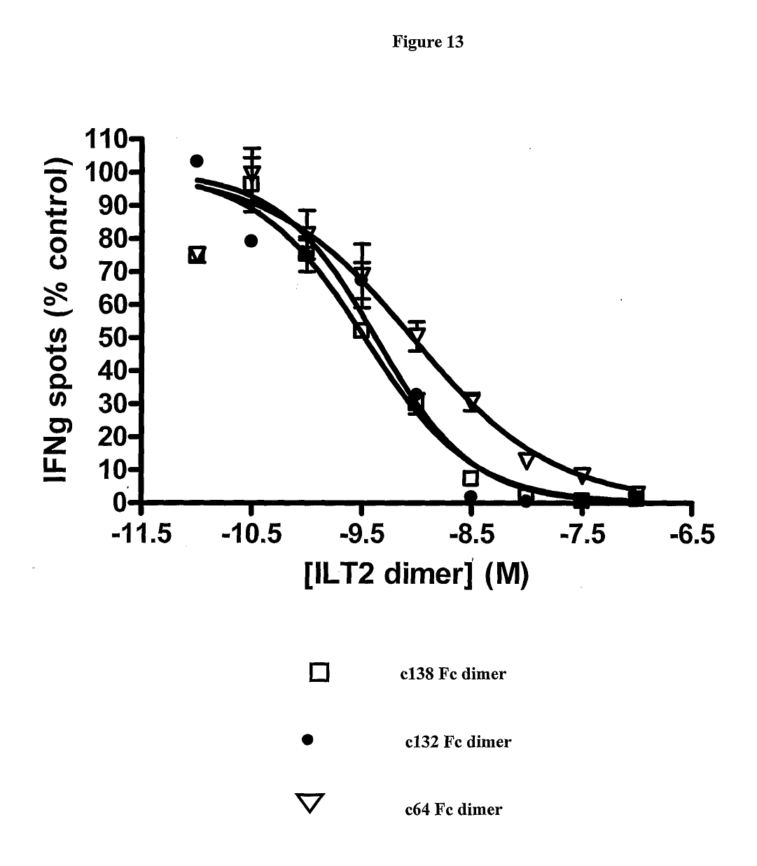

FIG. 13 is a graph of the effect of titrating the concentration of three ILT-FC fusion homodimers on the inhibition of T cell activation.

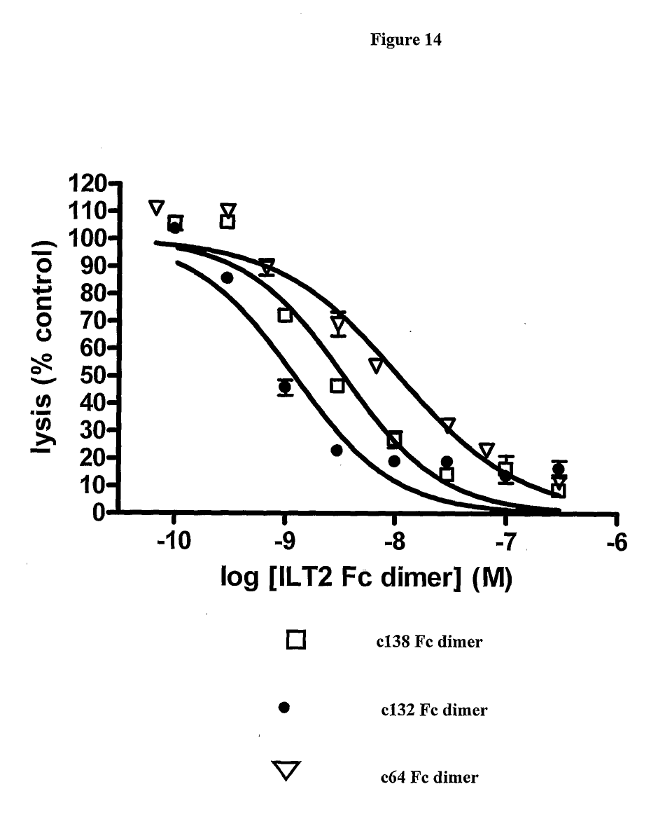

FIG. 14 is a graph of the effect of titrating the concentration of three ILT-FC fusion homodimers on the inhibition of T cell-mediated cell lysis

### EXAMPLE 1

### Production of a Soluble Wild-Type ILT-2 Molecule Comprising Domains 1 and 2

This examples details the production of a soluble wild-type ILT-2 molecule comprising domains 1 and 2 (D1D2) thereof.

FIG. 3 (SEQ ID NO: 5) provides the DNA sequence used to express a soluble wild-type ILT-2 containing only domains D1 and D2. This DNA sequence was synthesised de-novo by a contract research companies, GeneArt (Germany). Restriction enzyme recognition sites (NdeI and HindIII) have been introduced into this DNA sequence in order to facilitate ligation of the DNA sequence into a pGMT7-based expression plasmid, which contains the T7 promoter for high level expression in E. coli strain BL21-DE3(pLysS) (Pan et al., Biotechniques (2000) 29 (6): 1234-8)

This DNA sequence is ligated into a pGMT7 vector cut with NdeI and HindIII. (See FIG. 5 for the DNA sequence of this vector and FIG. 6 for the plasmid map of this vector).

Restriction Enzyme Recognition Sites as Introduced into DNA Encoding the Soluble Wild-Type ILT-2 Polypeptide

### NdeI—CATATG

### HindIII—AAGCTT

### Ligation

The cut ILT-2 DNA and cut vector are ligated using a rapid DNA ligation kit (Roche) following the manufacturers instructions.

Ligated plasmids are transformed into competent E. coli strain XL1-blue cells and plated out on LB/agar plates containing 100 mg/ml ampicillin. Following incubation overnight at 37° C., single colonies are picked and grown in 10 ml LB containing 100 mg/ml ampicillin overnight at 37° C. with shaking. Cloned plasmids are purified using a Miniprep kit (Qiagen) and the insert is sequenced using an automated DNA sequencer (Lark Technologies).

FIG. 2a shows the amino acid sequence of the soluble wild-type ILT-2 polypeptide produced from the DNA sequence of FIG. 2b.

This polypeptide is used as the reference polypeptide to compare the pMHC affinity and ability to inhibit CD8/pMHC binding of the high affinity ILT-like polypeptides which comprise the N-terminal segment of the first and/or second polypeptides of the polypeptides of the present invention. The methods required to carry out these determinations are detailed in Examples 4 and 5 respectively.

### EXAMPLE 2

### Production of High Affinity Variants of the Soluble Wild-Type ILT-2 Polypeptide

The soluble wild-type ILT-2 polypeptide produced as described in Example 1 can be used a template from which to produce the polypeptides of the invention which have an increased affinity and/or slower off-rate for class I pMHC molecules.

As is known to those skilled in the art the necessary codon changes required to produce these mutated chains can be introduced into the DNA encoding the soluble wild-type ILT-2 polypeptide by site-directed mutagenesis. (QuickChange™ Site-Directed Mutagenesis Kit from Stratagene)

Briefly, this is achieved by using primers that incorporate the desired codon change(s) and the plasmids containing the DNA encoding the soluble wild-type ILT-2 polypeptide as a template for the mutagenesis:

Mutagenesis was carried out using the following conditions: 50 ng plasmid template, 1 μl of 10 mM dNTP, 5 μl of 10× Pfu DNA polymerase buffer as supplied by the manufacturer, 25 pmol of fwd primer, 25 pmol of rev primer, 1 μl pfu DNA polymerase in total volume 50 μl. After an initial denaturation step of 2 mins at 95 C, the reaction was subjected to 25 cycles of denaturation (95 C, 10 secs), annealing (55 C 10 secs), and elongation (72 C, 8 mins). The resulting product was digested with DpnI restriction enzyme to remove the template plasmid and transformed into E. coli strain XL1-blue. Mutagenesis was verified by sequencing.

The amino sequences of the soluble (D1D2) mutated ILT-like polypeptides which demonstrate high affinity for the YLSGANLNL (SEQ ID NO: 138)—HLA-A*0201 complex are listed in FIGS. 4a to 4eb (SEQ ID Nos: 6 to 136). As is known to those skilled in the art the necessary codon changes required to produce these mutated polypeptides can be introduced into the DNA encoding the wild-type soluble ILT-2 polypeptide by site-directed mutagenesis. (QuickChange™ Site-Directed Mutagenesis Kit from Stratagene)

### EXAMPLE 3

### Expression, Refolding and Purification of Soluble ILT-Like Polypeptides

The expression plasmid containing the soluble ILT-like polypeptides as prepared in Examples 1 or 2 were transformed separately into E. coli strain rosetta DE3pLysS, and single ampicillin/chloramphenicol-resistant colonies were grown at 37° C. in TYP (ampicillin 100 μg/ml, chloramphenicol 15 μg/ml) medium for 7 hours before inducing protein expression with 0.5 mM IPTG. Cells were harvested 15 hours post-induction by centrifugation for 30 minutes at 4000 rpm in a Beckman J-6B. Cell pellets were resuspended in a buffer, re-suspended cells were sonicated in 1 minute bursts for a total of around 10 minutes in a Milsonix XL2020 sonicator using a standard 12 mm diameter probe. Inclusion body pellets were recovered by centrifugation for 10 minutes at 4000 rpm in a Beckman J2-21 centrifuge. Three detergent washes were then carried out to remove cell debris and membrane components. Each time the inclusion body pellet was homogenised in a Triton buffer (50 mM Tris-HCl, 0.5% Triton-X100, 200 mM NaCl, 10 mM NaEDTA, 0.1% (w/v) NaAzide, 2 mM DTT, pH 8.0) before being pelleted by centrifugation for 15 minutes at 4000 rpm in a Beckman J2-21. Detergent and salt was then removed by a similar wash in the following buffer: 50 mM Tris-HCl, 1 mM NaEDTA, 0.1% (w/v) NaAzide, 2 mM DTT, pH 8.0. Finally, the inclusion bodies were divided into 60 mg aliquots and frozen at −70° C. Inclusion body protein yield was quantitated by solubilising with 6M guanidine-HCl and measurement using a UV spectrometer.

Approximately 60 mg of ILT polypeptide solubilised inclusion bodies was thawed from frozen stocks and diluted into 15 ml of a guanidine solution (6 M Guanidine-hydrochloride, 10 mM Sodium Acetate, 10 mM EDTA), to ensure complete chain denaturation. The guanidine solution containing fully reduced and denatured ILT polypeptide was then injected into 1 litre of the following refolding buffer: 100 mM Tris pH 8.5, 400 mM L-Arginine, 2 mM EDTA, 5 mM reduced Cystaeimine, 0.5 mM 2-mercaptoethylamine, 5M urea. The redox couple (2-mercaptoethylamine and cystamine (to final concentrations of 6.6 mM and 3.7 mM, respectively) were added approximately 5 minutes before addition of the denatured ILT polypeptide. The solution was left for 30 minutes. The refolded ILT polypeptide was dialysed in Spectrapor 1 membrane (Spectrum; Product No. 132670) against 10 L 10 mM Tris pH 8.1 at 5° C.±3° C. for 18-20 hours. After this time, the dialysis buffer was changed to fresh 10 mM Tris pH 8.1 (10 L) and dialysis was continued at 5° C.±3° C. for another 20-22 hours.

Soluble ILT polypeptide was separated from degradation products and impurities by loading the dialysed refold onto a POROS 50HQ anion exchange column and eluting bound protein with a gradient of 0-500 mM NaCl over 50 column volumes using an Akta purifier (Pharmacia). Peak fractions were stored at 4° C. and analysed by Coomassie-stained SDS-PAGE before being pooled and concentrated. Finally, the soluble ILT polypeptide was purified and characterised using a Superdex 200HR gel filtration column pre-equilibrated in HBS-EP buffer (10 mM HEPES pH 7.4, 150 mM NaCl, 3.5 mM EDTA, 0.05% nonidet p40). The peak eluting at a relative molecular weight of approximately 27 kDa was pooled and concentrated prior to characterisation by Biacore surface plasmon resonance analysis.

### EXAMPLE 4

### Biacore Surface Plasmon Resonance Characterisation of the Binding of Soluble ILT-Like Molecules to pMHC Molecules

A surface plasmon resonance biosensor (Biacore 3000™) was used to analyse the binding of soluble high affinity ILT-like molecules to class I pMHC. Such polypeptides form the N-terminal segments of the first and/or second polypeptides of the polypeptide dimers of the invention. This analysis was facilitated by producing soluble biotinylated pMHC (described below) which were immobilised to a streptavidin-coated binding surface in a semi-oriented fashion, allowing efficient testing of the binding of a soluble ILT-like molecule to up to four different pMHC (immobilised on separate flow cells) simultaneously. Injection of the pMHC allows the precise level of immobilised class II molecules to be manipulated easily.

Soluble biotinylated class I HLA-A*0201 loaded with a CEA-derived YLSGANLNL (SEQ ID NO: 138) peptide were refolded in vitro from bacterially-expressed inclusion bodies containing the constituent subunit proteins and synthetic peptide, followed by purification and in vitro enzymatic biotinylation (O'Callaghan et al. (1999) Anal. Biochem. 266: 9-15). MHC-heavy chain was expressed with a C-terminal biotinylation tag which replaces the transmembrane and cytoplasmic domains of the protein in an appropriate construct. Inclusion body expression levels of ˜75 mg/litre bacterial culture were obtained. The MHC light-chain or β2-microglobulin was also expressed as inclusion bodies in E. coli from an appropriate construct, at a level of ˜500 mg/litre bacterial culture.

The E. coli cells were lysed and inclusion bodies are purified to approximately 80% purity. Protein from inclusion bodies was denatured in 6 M guanidine-HCl, 50 mM Tris pH 8.1, 100 mM NaCl, 10 mM DTT, 10 mM EDTA, and was refolded at a concentration of 30 mg/litre heavy chain, 30 mg/litre β2m into 0.4 M L-Arginine-HCl, 100 mM Tris pH 8.1, 3.7 mM cystamine, 6.6 mM β-cysteamine, 4 mg/ml of the peptide required to be loaded by the MHC, by addition of a single pulse of denatured protein into refold buffer at <5° C. Refolding was allowed to reach completion at 4° C. for at least 1 hour.

Buffer was exchanged by dialysis in 10 volumes of 10 mM Tris pH 8.1. Two changes of buffer were necessary to reduce the ionic strength of the solution sufficiently. The protein solution was then filtered through a 1.5 μm cellulose acetate filter and loaded onto a POROS 50HQ anion exchange column (8 ml bed volume). Protein was eluted with a linear 0-500 mM NaCl gradient. The soluble biotinylated HLA-A2-peptide complex eluted at approximately 250 mM NaCl, and peak fractions were collected, a cocktail of protease inhibitors (Calbiochem) was added and the fractions were chilled on ice.

Biotinylation tagged pMHC were buffer exchanged into 10 mM Tris pH 8.1, 5 mM NaCl using a Pharmacia fast desalting column equilibrated in the same buffer. Immediately upon elution, the protein-containing fractions were chilled on ice and protease inhibitor cocktail (Calbiochem) was added. Biotinylation reagents were then added: 1 mM biotin, 5 mM ATP (buffered to pH 8), 7.5 mM MgCl2, and 5 μg/ml BirA enzyme (purified according to O'Callaghan et al. (1999) Anal. Biochem. 266: 9-15). The mixture was then allowed to incubate at room temperature overnight.

Biotinylated pMHC were purified using gel filtration chromatography. A Pharmacia Superdex 75 HR 10/30 column was pre-equilibrated with filtered PBS and 1 ml of the biotinylation reaction mixture was loaded and the column was developed with PBS at 0.5 ml/min. Biotinylated pMHC eluted as a single peak at approximately 15 ml. Fractions containing protein were pooled, chilled on ice, and protease inhibitor cocktail was added. Protein concentration was determined using a Coomassie-binding assay (PerBio) and aliquots of biotinylated pMHC were stored frozen at −20° C. Streptavidin was immobilised by standard amine coupling methods.

Such immobilised pMHC are capable of binding soluble T-cell receptors and the coreceptor CD8αα, as well as ILT-like molecules, and these interactions can be used to ensure that the immobilised pMHC are correctly refolded.

The interactions between a soluble ILT-like molecule and CEA-derived YLSGANLNL (SEQ ID NO: 138)—HLA—A*0201, the production of which is described above, were analysed on a Biacore 3000™ surface plasmon resonance (SPR) biosensor. SPR measures changes in refractive index expressed in response units (RU) near a sensor surface within a small flow cell, a principle that can be used to detect receptor ligand interactions and to analyse their affinity and kinetic parameters. The probe flow cells were prepared by immobilising the pMHC complexes in flow cells via biotin-tag binding. The assay was then performed by passing soluble ILT-like molecule over the surfaces of the different flow cells at a constant flow rate, measuring the SPR response in doing so.

### To Measure Equilibrium Binding Constant

Serial dilutions of soluble ILT-like molecules were prepared and injected at constant flow rate of 5 μl min-1 over two different flow cells; one coated with ˜500 RU of the specific—HLA-A*0201 complex, the second cell was left blank as a control. Response was normalised for each concentration using the measurement from the control cell. Normalised data response was plotted versus concentration of ILT-like sample and fitted to a hyperbola in order to calculate the equilibrium binding constant, K_{D}. (Price & Dwek, Principles and Problems in Physical Chemistry for Biochemists (2^{nd} Edition) 1979, Clarendon Press, Oxford).

### To Measure Kinetic Parameters

For high affinity soluble ILT-like molecules K_{D} was determined by experimentally measuring the dissociation rate constant, kd, and the association rate constant, ka. The equilibrium constant K_{D} was calculated as kd/ka.

High affinity ILT-like molecules were injected over two different cells one coated with ˜300 RU of CEA-derived YLSGANLNL (SEQ ID NO: 138)—HLA-A*0201 complex, the second was left blank as a control Flow rate was set at 50 μl/min. Typically 250 μl of ILT polypeptide at ˜3 μM was injected. Buffer was then flowed over until the response had returned to baseline. Kinetic parameters were calculated using Biaevaluation software. The dissociation phase was also fitted to a single exponential decay equation enabling calculation of half-life.

### Results

The interaction between a soluble variant of wild-type ILT-2 and the CEA-derived YLSGANLNL (SEQ ID NO: 138)—HLA-A*0201 complex was analysed using the above methods and demonstrated a K_{D} of approximately 6 μM. The ILT-like molecules having the amino acid sequences provided in FIGS. 4a to 4eb (SEQ ID Nos: 6 to 136 have a K_{D} of less than or equal to 1 μM and/of 2 S⁻¹ or slower.

The interaction of a dimer of the invention comprising two high affinity ILT-like segments of SEQ ID NO: 19 and (a) a GP100-derived peptide-HLA-A*0201 complex and (b) a Teolmerase-derived peptide-HLA*2402 complex was analysed using the above methods. Interaction half-lives (t_{1/2}) of 159 hours and 194 hours respectively were observed for these interactions. See FIG. 11 for the Biacore trace used to calculate these figures.

### EXAMPLE 5

### Biacore Surface Plasmon Resonance Analysis of Soluble ILT-Mediated Inhibition of the pMHC/CD8 Interaction

A surface plasmon resonance biosensor (Biacore 3000™) is used to analyse the ability of soluble high affinity ILT-like molecules to mediate inhibition of the class I pMHC/CD8 interaction. Such polypeptides form the N-terminal segments of the first and/or second polypeptides of the polypeptide dimers of the invention. This analysis is facilitated by producing soluble pMHC complexes (described below) and biotinylated soluble CD8αα molecules (also described below). The biotinylated soluble CD8αα molecules are immobilised to a streptavidin-coated binding surface “Biacore chip” in a semi-oriented fashion, allowing efficient testing of the binding of soluble pMHC complexes to the immobilised soluble CD8αα. Injection of the biotinylated soluble CD8αα molecules allows the precise level of immobilised CD8 molecules to be manipulated easily.

Soluble HLA-A*0201 pMHC loaded with a CEA-derived YLSGANLNL (SEQ ID NO: 138) peptide are produced using the methods substantially as described in (Garboczi et. al., (1992) PNAS USA 89 3429-3433). The soluble pMHC molecules are refolded in vitro from E. coli expressed inclusion bodies containing the constituent subunit proteins and synthetic peptide and then purified. The MHC light-chain or in-microglobulin is also expressed as inclusion bodies in E. coli from an appropriate construct, at a level of ˜500 mg/litre bacterial culture.

E. coli cells are lysed and inclusion bodies are purified, and the over-expressed proteins are refolded and purified using the methods detailed in Example 4 except that the biotinylation steps are omitted.

Biotinylated soluble CD8 molecules are produced as described in Examples 1 and 6 of EP1024822. Briefly, the soluble CD8α containing a C-terminal biotinylation tag is expressed as inclusion bodies in E. coli and then purified and refolded to produce CD8αα homodimers containing a tag sequence that can be enzymatically biotinylated. (Schatz, (1993) Biotechnology NY 11: 1138-43). Biotinylation of the tagged CD8α molecules is then achieved using the BirA enzyme (O'Callaghan, et al. Anal Biochem 266(1): 9-15 (1999) Biotinylation reagents are: 1 mM biotin, 5 mM ATP (buffered to pH 8), 7.5 mM MgCl2, and 5 μg/ml BirA enzyme (purified according to O'Callaghan et al. (1999) Anal. Biochem. 266: 9-15). The mixture is then allowed to incubate at room temperature overnight.

The biotinylated sCD8αα is immobilised on the surface of a Biacore streptavidin-coated chip producing a change in the refractive index of 1000 response units. Such immobilised CD8αα molecules are capable of binding soluble pMHC complexes which may be injected in the soluble phase.

The ability of the ILT molecules to inhibit the pMHC/CD8 interaction on a Biacore 3000™ surface plasmon resonance (SPR) biosensor is analysed as follows:

SPR measures changes in refractive index expressed in response units (RU) near a sensor surface within a small flow cell, a principle that can be used to detect receptor ligand interactions and to analyse their affinity and kinetic parameters. The chips are prepared by immobilising the soluble biotinylated CD8αα molecules to streptavidin coated chips as described above. Serial dilutions of soluble wild-type ILT-2 (SEQ ID NO: 3) wild-type ILT or high affinity ILT-like molecules are prepared and injected at constant flow rate of 5 μl min⁻¹ over a flow cell coated with 1000 RU of biotinylated CD8αα in the presence of a suitable concentration of soluble YLSGANLNL (SEQ ID NO: 138)—HLA-A*0201. The inhibition of the SPR responses for the CD8αα/pMHC interaction produce a dose response curve which is used to calculate an IC50 value for the polypeptide being assayed for this interaction.

### EXAMPLE 6

### Comparison of Polypeptide Sequence Identity and Similarity

The protein-protein comparison algorithm used to generate identity and similarity data for this application is available via the following website:

http://fasta.bioch.virginia.edu/fasta_www/cgi/search_frm2.cgi

The “FASTA: protein: protein DNA: DNA” programme available on this website was used to carry out these comparisons. The following (default) settings were used:

### Ktup: Ktup=2

Scoring matrix: Blosum 50

### Gap: −10

### Ext: −2

In order to run the required comparisons either the soluble ILT-2 fragment amino acid sequence in single letter code as provided in FIG. 4o (SEQ ID NO: 19) or the wild-type human IgG1 Fc amino acid sequence in single letter code as provided in FIG. 7 (SEQ ID NO: 139) is entered as the first (query) sequence and the amino acid sequence for comparison thereto is entered as the second (library) sequence. The algorithm can then be run and will provide percentage identity and similarity scores for the pair of sequences compared.

As will be obvious to those skilled in the art there are a number of sources of FASTA protein: protein comparisons which could be used for this analysis.

### EXAMPLE 7

### Production of the Polypeptide Dimers of the Invention in Chinese Hamster Ovary (CHO) Cells

2 ug of a pFUSE vector DNA (Invivogen; pfuse-hg1fc2 or pfc2-hg1e3) was digested with BglII and EcoRI restriction enzymes for 4.5 h at 37° C. These vectors incorporate DNA encoding wild-type (pfuse-hg1fc2 vector) and mutated (pfc2-hg1e3 vector) FC portions of the human IgG1 immunoglobulin respectively. Digested vector DNA was purified using commercially available spin-columns. DNA encoding amino acids 3 to 198 of the following ILT2 clones using the numbering of SEQ ID NO: 3 were PCRed from template vector DNA using the forward primer SD113 (tagged with an EcoRI site) and the reverse primers SD114 and SD115 (tagged with BglII sites) which encode two different linkers differing in length by four amino acids.

Clone 64 (SEQ ID NO: 123 provides the full amino acid sequence of this polypeptide) and clone 83 (SEQ ID NO: 19 provides the full amino acid sequence of this polypeptide) clone 132 (SEQ ID NO: 131 provides the full amino acid sequence of this polypeptide).

SD113
5′-cacttgtcacgaattcgcatcttccaaaaccWactctctgggctg-
3′
SD114
5′-agttttgtcagatctcgatgggtccattcgtccatcgacatcgagct
ccaggagatc-3′
SD115
5′-agttttgtcagatctcgatggatcgacatcgagctccaggagatc-
3′

The PCR products were digested with EcoRI and BglII for 3 hours at 37° C. and the digested fragments were gel-purified using a commercially available kit.

The ILT clone-linker fragments were ligated into the digested pFUSE vectors and transformed into E. coli strain XL-1 Blue. Following selection of transformed clones on solid media containing 100 ug/ml zeocin, DNA was isolated for sequencing using a commercially available kit. Clones of the correct sequence were grown in 50 ml LB media and Fc-fusion vector DNA isolated for cell transfections using a commercially available kit.

Transfections of log-phase CHO-S suspension cells (Invitrogen) growing in serum-free CD-CHO medium (Invitrogen) with the ILT2:pFUSE constructs were performed using Lipofectamine 2000 reagent according to the manufacturers instructions. Transfected cultures were grown under zeocin selection (400 ug/ml) for 3-4 weeks to generate stable polyclonal lines. The ILT-Fc-fusion polypeptides secreted from polyclonal lines were purified using Protein A affinity resin according to standard protocols. The isolation of high-expressing discrete clones was performed by FACS seeding single cells into 96-well plates containing 200 ul of serum-free medium per well and 400 ug/ml zeocin.

FIG. 10 is a gel of a polypeptide dimer Fc fusion of the invention produced using the above method which comprises two Clone c83 ILT-like segments having the amino acid sequence of SEQ ID NO: 19. This gel was run under reducing and non-reducing conditions.

### EXAMPLE 8

### Competitive Binding Fluorescence Activated Cell Sorting (FACS) Assay for Assessing the Ability of the Polypeptide Monomers or Dimers of the Invention to Bind to Fc Receptors

In order to carry a FACS-based assessment of the ability of the polypeptide monomers or dimers of the invention to bind to a given Fc receptor a cell-line expressing the required FC receptor has to be obtained or produced.

Hinton et al., (2004) J Biol. Chem. 279 (8): 6213-6216 details the methods required to obtain a suitable cell line, and to carry out an appropriate FACS-based assay for assessing the ability of the polypeptide monomers or dimers of the invention to bind to the human neo-natal Fc receptor (FcRn).

Briefly, cDNA encoding the human FcRn and human beta-2 microglobulin is cloned by PCR from peripheral blood monucleate cells (PBMCs) and sub-cloned cloned into a vector derived from pVk. The NS0 mouse myeloma cell line (The European Collection of Animal Cell Cultures, Salisbury, UK) is then transfected with this vector by electroporation to obtain a stably transfected cell line.

FACS-based competitive binding assays can then be carried by analysing the ability of the polypeptide monomers or dimers of the invention to compete against the binding of a range of concentrations of a reference human IgG antibody to FcRn. Any reduction in the observed level of binding of the reference antibody in the presence of the polypeptide monomers or dimers of the invention would indicate that polypeptides were capable of binding to human FcRn.

### EXAMPLE 9

### ELISPOT Assay for Assessing In-Vitro Inhibition of Cyto-Toxic T Cell (CTL) Activation by the Polypeptide Monomers or Dimers of the Invention

The following method provides a means of assessing the ability of the polypeptide monomers or dimers of the invention to inhibit CD8 co-receptor mediated T cell activation.

### Reagents:

Assay media: 10% FCS (heat-inactivated, Gibco, cat# 10108-165), 88% RPMI 1640 (Gibco, cat# 42401-018), 1% glutamine (Gibco, cat# 25030-024) and 1% penicillin/streptomycin (Gibco, cat# 15070-063).

Wash buffer: 0.01 M PBS/0.05% Tween 20 (1 sachet of Phosphate buffered saline with Tween 20, pH 7.4 from Sigma, Cat. # P-3563 dissolved in 1 litre distilled water gives final composition 0.01 M PBS, 0.138 M NaCl, 0.0027 M KCl, 0.05% Tween 20).

PBS (Gibco, cat#10010-015).

Diaclone EliSpot kit (IDS) EliSpot kit contains all other reagents required i.e. capture and detection antibodies, skimmed milk powder, BSA, streptavidin-alkaline phosphatase, BCIP/NBT solution (Human IFN-γ PVDF Eli-spot 20×96 wells with plates (IDS cat# DC-856.051.020, DC-856.000.000.

The following method is based on the manufacturers instructions supplied with each kit but contains some alterations.

### Method

100 μl capture antibody was diluted in 10 ml sterile PBS per plate. 100 μl diluted capture antibody was aliquoted into each well and left overnight at 4° C., or for 2 hr at room temperature. The plates were then washed three times with 450 μl wash buffer, Ultrawash 96-well plate washer, (Thermo Life Sciences) to remove excess capture antibody. 100 μl of 2% skimmed milk was then added to each well. (One vial of skimmed milk powder as supplied with the EliSpot kit is dissolved in 50 ml sterile PBS). The plates were then incubated at room temperature for two hours before washing washed a further three times with 450 μl wash buffer, Ultrawash 96-well plate washer, (Thermo Life Sciences)

Mel 624 target cells were detached from their tissue culture flasks using trypsin, washed once by centrifugation (280×g for 10 minutes) in assay media and resuspended at 1×10⁶/ml in the same media. 50 ul of this suspension was then added to the assay plate to give a total target cell number of 50,000 cells/well.

A MART-1 specific T cell clone (KA/CS) (effector cell line) was harvested by centrifugation (280×g for 10 min) and resuspended at 1×10⁴/ml in assay media to give 500 cells/well when 50 μl was added to the assay plate.

The polypeptide monomer or dimer of the invention was diluted in assay media at a 3× concentration to give a 1× final when 50 ul is added to the plate in a final volume of 150 μl. A range of different concentration solutions of this test sample were then prepared for testing.

Wells containing the following were then prepared, (the final reaction volume in each well is 150 μl):

### Test Samples (Added in Order)

50 μl Mel 624 target cells

50 ul of the desired concentration of the polypeptide monomer or dimer.

50 ul T cell clone effector cells.

### Negative Controls

50 μl target cells.

50 ul of the highest concentration the polypeptide monomer or dimer.

50 μl assay media.

### OR

50 μl effector cells.

50 μl of the highest concentration of the polypeptide monomer or dimer.

50 μl assay media

### Positive Controls

50 μl Mel 624 target cells

50 μl effector cells

50 μl assay media

### OR

To show MHC class I dependency

50 μl Mel 624 target cells

50 μl effector cells

50 μl containing 100 μg/ml W6/32 anti MHC class I antibody

The plates were then incubated overnight at 37° C./5% CO₂. The plates were then washed six times with wash buffer before tapping out excess buffer. 550 μl distilled water was then added to each vial of detection antibody supplied with the ELISPOT kit to prepare a diluted solution. 100 μl of the diluted detection antibody solution was then further diluted in 10 ml PBS/1% BSA per plate and 100 μl of the diluted detection antibody solution was aliquoted into each well. The plates were then incubated at room temperature for 90 minutes.

After this time the plates were washed three times with wash buffer (three times with 450 μl wash buffer, Ultrawash 96-well plate washer (Thermo Life Sciences) and tapped dry. 10 μl streptavidin-Alkaline phosphatase was then diluted with 10 ml with PBS/1% BSA per plate and 100 μl of the diluted streptavidin was added to each well and incubated at room temperature for 1 hr. The plates were then washed again three times with 450 μl wash buffer and tapped dry.

100 μl of the BCIP/NBT supplied solution was added to each well and the plates were covered in foil and left to develop for 2-5 min. The plates were checked regularly during this period for spot formation in order to decide when to terminate the reaction.

The plates were then washed thoroughly in tap water and shaken before being taken apart and left to dry on the bench.

Once dry the plates were read using an ELISPOT reader (Autoimmune Diagnotistika, Germany).

The number of spots that appeared in each well is proportional to the number of T cells activated. Therefore, any decrease in the number of spots in the wells containing the polypeptide monomer or dimer indicates inhibition of CD8 co-receptor-mediated CTL activation.

### Results

FIG. 13 is a graph of the effect of titrating the concentration of three ILT-Fc fusion homodimers on the inhibition of T cell activation. The “c64 Fc dimer” is an ILT Fc fusion homodimer of the invention. SEQ ID NO: 158 (FIG. 9o) provides the full amino acid sequence of the polypeptide monomer of this homodimer which comprises amino acids 3-198 of the mutated human ILT molecule of SEQ ID NO: 123. (FIG. 4do) This c64 ILT-Fc fusion comprises amino acids 3 to 198 of the mutated human ILT molecule of FIG. 13bd of WO 2006/125963. The “c132 Fc dimer” is an ILT Fc fusion homodimer of the invention. SEQ ID NO: 166 (FIG. 9w) provides the full amino acid sequence of the polypeptide monomer of this homodimer which comprises amino acids 3-198 of the mutated human ILT molecule of SEQ ID NO: 131. (FIG. 4dw)

The “c138 Fc dimer” is a member of the class of polypeptide homodimer Fc fusions. SEQ ID NO: 169 (FIG. 12b) provides the full amino acid sequence of the polypeptide monomer of this homodimer which comprises amino acids 1 to 196 of the mutated human ILT molecule of SEQ ID NO: 168 (FIG. 12a).

These data presented in FIG. 13 demonstrate that all three of the ILT-Fc fusions are capable of inhibiting the activation of T cells. (c138 Fc dimer IC₅₀=0.4 nM±0.08 SEM (n=10), c132 Fc dimer IC₅₀=0.45 nM±0.02 SEM (n=3) and c64 Fc dimer IC₅₀=2.3 nM±1.0 SEM (n=13))

### EXAMPLE 10

### In-Vitro Cellular Assay of T Cell Mediated Target Cell Lysis in the Presence and Absence of Polypeptide Monomers or Polypeptide Dimers of the Invention

Target cells (Mel 624 or peptide pulsed T2 cells) were loaded with BATDA reagent for 30 min at 37° C./5% CO2 according to package instructions (1-3 μl BATDA added to 1×10⁶ cells in 1 ml assay media). The target cells were washed three times in assay media containing 100 μM β-mercaptoethanol and resuspended at 1×10⁵ cells/ml to give 5000 cells/well in 50 μl. The ILT-Fc fusion polypeptide dimers were added to the wells at varying concentrations (50 μl of 3× final concentration in assay media) before the addition of effector cells (T cell clones, Melc5 or EBV 176 D5.1). The effector to target ratio was determined for each T cell clone (3:1 Melc5:Mel 624;) and the relevant number of effector cells was added in 500 assay media. Target cells alone (spontaneous release), target cells+1% triton (maximum release) and the supernatant from the final wash of the targets (background) were used as assay controls. The plates were incubated at 37° C./5% CO2 for 2 hours. The plates were centrifuged and 20 μl of supernatant was transferred to a black plate. 180 μl europium solution was added to each well and the plates were shaken for 15 min before reading in the Wallac Victor II.

% Spontaneous release=100×(spotaneous release-background)/(maximum release-background)

% Specific lysis=100×(experimental release−spontaneous release)/(maximum release−spontaneous release)

### Results

Any reduction in the percentage cell lysis observed in the sample wells containing the ILT-Fc fusion dimers compared to percentage cell lysis observed in the control wells indicates that the ILT-Fc fusion dimers are causing an inhibition of CD8⁺ T cell-mediated target cell lysis.

FIG. 14 is a graph of the effect of titrating the concentration of three ILT-Fc fusion homodimers on the inhibition of T cell-mediated cell lysis. The “c64 Fc dimer” is an ILT Fc fusion homodimer of the invention. SEQ ID NO: 158 (FIG. 9o) provides the full amino acid sequence of the polypeptide monomer of this homodimer which comprises amino acids 3-198 of the mutated human ILT molecule of SEQ ID NO: 123. (FIG. 4do) This c64 ILT-Fc fusion comprises amino acids 3 to 198 of the mutated human ILT molecule of FIG. 13bd of WO 2006/125963. The “c132 Fc dimer” is an ILT Fc fusion homodimer of the invention. SEQ ID NO: 166 (FIG. 9w) provides the full amino acid sequence of the polypeptide monomer of this homodimer which comprises amino acids 3-198 of the mutated human ILT molecule of SEQ ID NO: 131. (FIG. 4dw)

The “c138 Fc dimer” is a member of the class of polypeptide homodimer Fc fusions. SEQ ID NO: 169 (FIG. 12b) provides the full amino acid sequence of the polypeptide monomer of this homodimer which comprises amino acids 1 to 196 of the mutated human ILT molecule of SEQ ID NO: 168 (FIG. 12a).

These data presented in FIG. 14 demonstrate that all three ILT-Fc fusions are effective at inhibiting T cell-mediated cell lysis. (c138 Fc dimer IC₅₀=2.1 nM±0.31 SEM (n=5), c132 Fc dimer IC₅₀=1.2 nM±0.32 SEM (n=2) and c64 Fc dimer IC₅₀=14.4 nM 5.3±SEM (n=13))

1. A polypeptide comprising an ILT-like segment and an Fc-like segment wherein either
(a) the ILT-like segment is the N-terminal segment of the polypeptide and has at least a 45% identity and/or 55% similarity to SEQ ID NO: 19; and said N-terminal segment per se has the property of (i) binding to a given Class I pMHC with a KD of less than or equal to 1 μM and/or with an off-rate (k_{off}) of 2 S⁻¹ or slower, and (ii) inhibiting CD8 binding to the given pMHC to a greater extent than the polypeptide SEQ ID NO: 3; and the Fc-like segment is the C-terminal segment of the polypeptide and comprises a portion of the constant domain of one of the heavy chains of an immunoglobulin having at least 70% identity and/or 80% similarity to the corresponding portion of SEQ ID NO 139; or
(b) the Fc-like segment is the N-terminal segment of the polypeptide and comprises a portion of the constant domain of one of the heavy chains of an immunoglobulin having at least 70% identity and/or 80% similarity to the corresponding portion of SEQ ID 139; and the ILT-like segment is the C-terminal segment of the polypeptide and has at least a 45% identity and/or 55% similarity to SEQ ID NO: 19; and said C-terminal segment per se has the property of (i) binding to a given Class I pMHC with a KD of less than or equal to 1 μM and/or with an off-rate (k_{off}) of 2 S⁻¹ or slower, and (ii) inhibiting CD8 binding to the given pMHC to a greater extent than the polypeptide SEQ ID NO: 3,
wherein the polypeptide is arranged as a monomeric polypeptide or as a polypeptide dimer comprising a first polypeptide and a second polypeptide, in which dimer
(i) the first and/or the second polypeptide comprises an ILT-like segment having at least a 45% identity and/or 55% similarity to SEQ ID NO: 19;
(ii) said ILT-like segment(s) per se have the property of (a) binding to a given Class I pMHC with a KD of less than or equal to 1 μM and/or with an off-rate (k_{Off}) of 2 S⁻¹ or slower, and (b) inhibiting CD8 binding to the given pMHC to a greater extent than the polypeptide SEQ ID NO: 3;
(iii) each of the first and second polypeptides comprises an Fc-like segment comprising a portion of the constant domain of one of the heavy chains of an immunoglobulin having at least 70% identity and/or 80% similarity to the corresponding portion of SEQ ID NO: 139;
and wherein either (a) the ILT-like segment(s) is/are the N-terminal segment(s) of the first and/or second polypeptides, and the Fc-like segments are the C-terminal segments of the first and second polypeptides or (b) the Fc-like segments are the N-terminal segments of the first and/or second polypeptides, and the ILT-like segment(s) is/are the C-terminal segment(s) of the first and second polypeptides.

2-3. (canceled)

4. The polypeptide dimer as claimed in claim 1, wherein said polypeptide is a dimer comprising at least one inter-chain covalent link between a residue in one of the said Fc-like segments and a residue in the other said Fc-like segment.

5-9. (canceled)

10. The polypeptide as claimed in claim 1, wherein the Fc-like segment or segments comprise respectively one or two of amino acid sequence SEQ ID NO: 139.

11. The polypeptide as claimed in claim 1, wherein the Fc-like segments or segments comprise respectively one or both of the chains of a mutated Fc portion of an immunoglobulin.

12. The polypeptide as claimed in claim 11, wherein the said Fc-like segment or segments is/are mutated so as to reduce antibody-dependent cellular cyto-toxicity (ADCC) and/or complement-dependent cellular cyto-toxicity (CDCC) responses to the monomer or dimer.

13. The polypeptide as claimed in claim 12 wherein the said Fc-like segment or segments has or have a sequence or sequences corresponding to SEQ ID NO: 139 in which one or more of amino acids corresponding to amino acids 13E, 14L, 15L, 16G, 107A, 110A, or 11 IP of SEQ ID NO: 139 is/are mutated.

14. The polypeptide as claimed in claim 12 wherein the said Fc-like segment or segments has or have a sequence or sequences corresponding to SEQ ID NO: 139 having one or more of the following mutations 13E→P, 14L→V, 15L→A, deletion of 16G, 107A→G, 110A→S or 11 lP→S using the numbering of SEQ ID NO: 139.

15. (canceled)

16. The polypeptide as claimed in claim 11 wherein the said Fc-like segment or segments has or have a sequence or sequences corresponding to SEQ ID NO: 139 in which one or both of amino acids corresponding to amino acids 30T and 208M of SEQ ID NO: 139 is/are mutated.

17. The polypeptide as claimed in claim 11 wherein the said Fc-like segment or segments has or have a sequence or sequences corresponding to SEQ ID NO: 139 having one or more of the following mutations 30T→Q or 208M→L using the numbering of SEQ ID NO: 139.

18. The polypeptide as claimed in claim 11, wherein the Fc-like segment, or both Fc-like segments, comprise the amino acid sequence of any of SEQ ID NOs 140 to 143.

19. The polypeptide as claimed in claim 1, wherein one or more amino acids of the ILT-like segment or segments corresponding to amino acids 1OW, 19Q, 2OG, 21S, 23V, 35E, 42K, 47W, 50R, 66I, 77Y, 78Y, 79G, 80S, 81D, 82T, 83A, 84G, 85R, 87E, 99A, 101I, 102K, 126Q, 127V, 128A, 129F, 130D, 141E, 146L, 147N, 1591, 168S, 170R, 172W, 174R, 179D, 180S, 181N, 182S, 187S, 188L, 189P, 196L or 198L of SEQ ID NO: 3 is/are mutated.

20. The polypeptide as claimed in claim 19, wherein the ILT-like segment, or both ILT-like segments, comprise one or more of the following mutations lOW→L, 19Q→M, 19Q→L, 19Q→V, 20G→D, 20G→M, 20G→Q, 20G→F, 20G→S, 20G→E, 20G→R, 21S→Q, 21S→R, 21S→A, 21S→S, 23V→L, 35E→Q, 42K→R, 47W→Q, 50R→L, 66L→V, 77Y→V, 77Y→M, 77Y→I, 77Y→Q, 78Y→Q, 78Y→I, 78Y→G, 79G→Q, 79G→Y, 79G→W, 79G→R, 79G→V, 80S→R, 80S→T, 80S→G, 81D→G, 81D→Q, 81D→L, 81D→V, 82T→G, 82T→E, 83A→S, 83A→G, 83A→R, 84G→L, 84G→Q, 84G→A, 85R→W, 87E→A, 99A→I, 99A→Y, 10H→L, 10H→K, 10H→Q, 101→V, 102K→Q, 102K→A, 102K→R, 126Q→P, 126Q→M, 127V→W, 127V→F, 128A→D, 128A→S, 128A→T, 128A→Y, 128A→V, 128A→L, 128A→Q, 128A→I, 129F→A, 129F→T, 129F→S, 129F→V, 130D→E, 141E→G, 141E→D, 146L→D, 147N→S, 159I→E, 168S→G, 170R→K, 172W→R, 174R→W, 179D→P, 179D→V, 179D→M, 179D→T, 179D→G, 180S→I, 180S→A, 180S→N, 180S→D, 180S→W, 180S→R, 180S→E, 181N→W, 181N→F, 181N→Y, 182S→T, 182S→A, 182S→W, 182S→F, 182S→L, 187S→T 188L→D, 188L→R, 188L→S, 188L→T, 188L→Q, 189P→G, 189P→M, 189P→S, 196L→D or 198L→D using the numbering of SEQ ID NO: 3.

21. (canceled)

22. The polypeptide as claimed in claim 19, wherein the ILT-like segment, or both ILT-like segments, comprise mutations corresponding to 19Q→M, 20G→D, 21S→Q, 83A→S, 84G→Q, 85R→W, 87E→A, 99A→V, 179D→M, 181N→W, 182S→A, 196L→D and 198L→D using the numbering of SEQ ID NO: 3.

23. (canceled)

24. The polypeptide as claimed in claim 19, wherein the ILT-like segment, or both ILT-like segments, comprise mutations corresponding to 19Q→M, 20G→D, 21S→Q, 35E→Q, 83A→R, 84G→Q, 85R→W, 87E→A, 99A→V, 141E→D, 196L→D and 198L→D using the numbering of SEQ ID NO: 3.

25-26. (canceled)

27. The polypeptide as claimed in claim 1 comprising any one of the polypeptide monomer sequences of SEQ ID NOs: 144 to 167.

28. The polypeptide as claimed in claim 27 comprising a polypeptide monomer selected from the group consisting of: SEQ ID NO: 150, SEQ ID NO: 158, and SEQ ID NO: 166.

29. (canceled)

30. The polypeptide as claimed in claim 1 comprising a C-terminal reactive site for covalent attachment of a desired moiety.

31. The polypeptide as claimed in claim 30, wherein said reactive site is a cysteine residue.

32. The polypeptide as claimed in claim 1 which is associated with at least one polyalkylene glycol chain(s).

33-34. (canceled)

35. The polypeptide as claimed in claim 30 which is covalently linked to a therapeutic agent or detectable label.

36-39. (canceled)

40. A multivalent complex comprising at least two polypeptide monomers and/or dimers as claimed in claim 1.

41-48. (canceled)

49. A pharmaceutical composition comprising a polypeptide as claimed in claim 1 which may be arranged as a monomer, dimer, or multivalent complex, together with a pharmaceutically acceptable carrier.

50. A method of treating an autoimmune disease, comprising administering to a subject a polypeptide as claimed in claim 1 which may be arranged as a monomer, dimer, or multivalent complex.

51. The method as claimed in claim 50 wherein the said autoimmune disease is Diabetes, Goodpasture's syndrome, Multiple sclerosis, Psoriasis, Rheumatoid arthritis, Myositis, Ankylosing spondylitis, Artery aneurysms in acute Kawasaki disease, Hashimoto's disease or Crohn's disease.

52. A method of treating asthma, eczema, allograft rejection, graft-versus-host disease, hepatitis, or cerebral malaria, comprising administering to a subject of a polypeptide as claimed in claim 1 which may be arranged as a monomer, dimer, or multivalent complex.

53-57. (canceled)
